(19) United States
(12) Patent Application Publication
(10) Pub. No.: US 2009/0137596 A1
(43) Pub. Date: May 28, 2009

(54) G-PROTEIN INHIBITOR

(75) Inventors: Gregoire PREVOST, Antony (FR); Marie-Christine Brezak Pannetier, Antony (FR); Christian Diolez, Palaiseau (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques (S.C.R.A.S.), Paris (FR)

(21) Appl. No.: 12/247,072

(22) Filed: Oct. 7, 2008

Related U.S. Application Data
(62) Division of application No. 10/562,625, filed on Mar. 1, 2006, now abandoned.

(30) Foreign Application Priority Data
Jun. 25, 2003 (FR) 03/07649

(51) Int. Cl. A61K 31/4985; C07D 487/04; A61P 35/00

(52) U.S. Cl. 514/249; 544/350

(57) ABSTRACT

A subject of the invention is a product comprising at least one Cdc25 phosphatase inhibitor in combination with at least one other anti-cancer agent for a therapeutic use which is simultaneous, separate or spread over time in the treatment of cancer.

According to the invention, the other anti-cancer agent is preferably chosen from:
analogues of DNA bases such as 5-fluorouracil;
Type I and/or II topoisomerase inhibitors such as for example camptothecin and its analogues, doxorubicin or amsacrine;
compounds interacting with the cell spindle such as for example paclitaxel (Taxol); compounds acting on the cytoskeleton such as vinblastine;
inhibitors of the transduction of the signal passing through the heterotrimeric G proteins;
prenyltransferase inhibitors, and in particular farnesyltransferase inhibitors;
cyclin-dependent kinase (CDKs) inhibitors;
alkylating agents such as cisplatin;
antagonists of folic acid such as methotrexate; and
inhibitors of the synthesis of DNA and cell division cell such as mitomycin C.

A further subject of the invention is (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine, or a pharmaceutically acceptable salt thereof, useful as an anticancer agent.

A subject of the present invention is a product comprising at least one Cdc25 phosphatase inhibitor in combination with at least one other anti-cancer agent for a therapeutic use which is simultaneous, separate or spread over time in the treatment of cancer.

Control of the transition between the different phases of the cell cycle during mitosis or meiosis is ensured by a group of proteins the enzyme activities of which are associated with different states of phosphorylation. These states are controlled by two large classes of enzymes: kinases and phosphatases.

Synchronization of the different phases of the cell cycle thus allows reorganization of the cell architecture at each cycle in the whole of the living world (microorganisms, yeast, vertebrates, plants). Among the kinases, the cyclin-dependent kinases (CDKs) play a major role in this control of the cell cycle. The enzyme activity of these different CDKs is controlled by two other families of enzymes which work in opposition (Jessus and Ozon, Prog. Cell Cycle Res. (1995), 1, 215-228). The first includes kinases such as Wee1 and Mik1 which deactivate the CDKs by phosphorylating certain amino acids (Den Haese et al., Mol. Biol. Cell (1995), 6, 371-385). The second includes phosphatases such as Cdc25 which activate the CDKs by dephosphorylating tyrosine and threonine residues of CDKs (Gould et al., Science (1990), 250, 1573-1576).

The phosphatases are classified in 3 groups: the serine/threonine phosphatases (PPases), the tyrosine phosphatases (PTPases) and the dual-specificity phosphatases (DSPases). These phosphatases play an important role in the regulation of numerous cell functions.

As regards human Cdc25 phosphatases, 3 genes (Cdc25-A, Cdc25-B and Cdc25-C) code for the Cdc25 proteins. Moreover, variants originating from alternative splicing of the Cdc25 genes have been identified (cf. for example Baldin et al., Oncogene (1997), 14, 2485-2495).

The role of the Cdc25 phosphatases in oncogenesis is now better known and the action mechanisms of these phosphatases are illustrated in particular in the following references: Galaktionov et al., Science (1995), 269, 1575-1577; Galaktionov et al., Nature (1996), 382, 511-517; and Mailand et al., Science (2000), 288, 1425-1429.

In particular, the overexpression of the different forms of Cdc25 is now reported in numerous series of human tumours:
Breast cancer: cf. Cangi et al., Résumé 2984, AACR meeting San Francisco, 2000);
Lymphomas: cf. Hernandez et al., Int. J. Cancer (2000), 89, 148-152 and Hernandez et al., Cancer Res. (1998), 58, 1762-1767;
Cancers of the neck and head: cf. Gasparotto et al., Cancer Res. (1997), 57, 2366-2368.

Moreover, E. Sausville's group reports an inverse correlation between the level of expression of Cdc25-B in a panel of 60 lines and their sensitivities to CDK inhibitors, suggesting that the presence of Cdc25 can bring a resistance to certain antineoplastic agents and more particularly to CDK inhibitors (Hose et al., Proceedings of AACR, Abstract 3571, San Francisco, 2000).

Among other targets, the pharmaceutical industry is therefore at present researching compounds capable of inhibiting the Cdc25 phosphatases in order to use them in particular as anti-cancer agents.

The invention relates to a product comprising at least one Cdc25 phosphatase inhibitor in combination with at least one other anti-cancer agent for a therapeutic use which is simultaneous, separate, or spread over time in the treatment of cancer.

Preferably, the invention will relate to a product comprising a Cdc25 phosphatase inhibitor in combination with at least one other anti-cancer agent for a therapeutic use which is simultaneous, separate or spread over time in the treatment of cancer.

By simultaneous therapeutic use, is meant in the present Application an administration of several active ingredients by the same route and at the same moment. By separate use, is meant in particular an administration of several active ingredients at approximately the same moment by different routes. By therapeutic use spread over time, is meant an administration of several active ingredients at different times and in particular an administration method according to which the administration of one of the active ingredients is carried out in its entirety before the administration of the other or others begins. One of the active ingredients can thus be administered over several months before administering the other active ingredient or the other active ingredients. There is no simultaneous treatment in this case.

According to the invention, the anti-cancer agent combined with the Cdc25 phosphatase is preferably such that it acts according to a route other than the Cdc25 phosphatases. In particular, said combined anti-cancer agent will have an inhibitory concentration IC₅₀ of at least 50 μM relative to the Cdc25 phosphatases or will have another activity with a IC₅₀ dose at least 10 times weaker relative to that of the Cdc25 phosphatases. Preferably, the combination produced according to the invention is such that it presents a synergy.

According to the invention, the Cdc25 phosphatase inhibitor is preferably chosen from derivatives of benzothiazole-4,7-diones and benzooxazole-4,7-diones corresponding to general formula (I) defined below.

A certain number of derivatives of benzothiazole-4,7-diones and of benzooxazole-4,7-diones are already known.

In particular, the patent GB 1 534 275 relates to herbicides the active ingredient of which is a compound corresponding to one of the general formulae

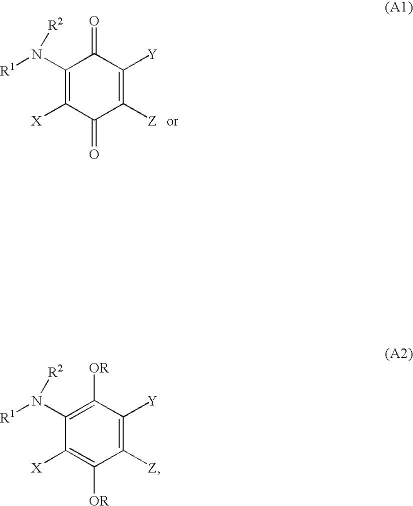

in which:

R¹ represents in particular a hydrogen atom or an alkyl or cycloalkyl radical;

R² represents in particular a hydrogen atom, an alkyl or cycloalkyl radical;

X represents in particular a halogen atom or an alkoxy radical;

Y and Z can in particular represent together with the carbon atoms which carry them a thiazole ring optionally substituted by an alkyl radical; and

R represents in particular an alkyl radical.

Moreover, the PCT Patent Application WO 99/32115 describes the compounds of general formula (A3)

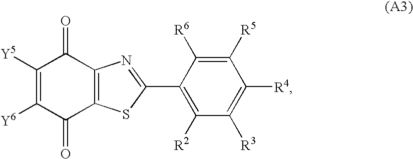

in which:

the substituents R²—R⁶ are chosen from the group constituted by a hydrogen atom, electron donor substituents, electron attractor substitutents and electron modulator substituents;

and Y⁵ and Y⁶ are in particular chosen from the group constituted by a hydrogen atom, electron donor substituents, electron attractor substitutents and electron modulator substituents.

In the PCT Patent Application WO 99/32115, the term “electron-donor substituent” refers to a functional group having a tendency to donate electron density; the substituents alkyl, alkenyl and alkynyl are mentioned. In this patent application, “electron-attracting substituents” always refers to a functional group having a tendency to attract electron density; the cyano, acyl, carbonyl, fluoro, nitro, sulphonyl and trihalomethyl substituents are mentioned. Finally, an “electron-modulating substituent” is defined in this Application as a functional group having a tendency to modulate the electron density, which can both attract and donate electrons and is therefore such that it can stabilize a cationic intermediate in an aromatic electrophilic substitution reaction; a functional group is mentioned, including, for example, amino (for example —NH₂, alkylamino or dialkylamino), hydroxy, alkoxy or aryl substituents, heterocyclic substituents, halogen atoms, etc.

The compounds of general formula (A3) are presented as modulators of the ryanodine receptors which can be used as pesticides or as therapeutic agents, for example in the treatment of congestive heart failure, migraines, hypertension, Parkinson's disease or Alzheimer's disease or in the prevention of miscarriage.

Finally, the derivatives of benzooxazole-4,7-diones of general formula (A4)

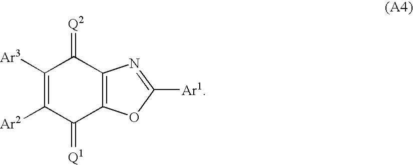

in which:

Ar¹ represents an optionally substituted aryl radical,

each of Ar² and Ar³ represents a hydrogen atom or an optionally substituted aryl radical, and

each of Q¹ and Q² represents in particular O,

are described as active constituents of light-sensitive layers of photo receptors.

In the PCT Patent Application FR02/04544, the Applicant described the compounds corresponding to the general formula (I)

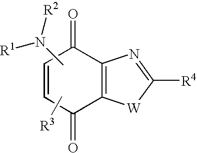

in which:

R¹ represents a hydrogen atom or an alkyl, alkoxyalkyl, alkylthioalkyl, cycloalkyl, —(CH₂)—X—Y, —(CH_{z})-Z-NR⁵R⁶ radical or a —CHR³⁵R³⁶ radical in which R³⁵ and R³⁶ form together with the carbon atom which carries them an indanyl or tetralinyl radical, or also R³⁵ and R³⁶ form together with the carbon atom which carries them a saturated heterocycle containing 5 to 7 members and from 1 to 2 heteroatoms chosen from O, N and S, the nitrogen atoms of said heterocycle being optionally substituted by radicals chosen from the alkyl radicals and the benzyl radical,

R¹ also being able, when W represents O, to represent moreover a carbocyclic aryl radical optionally substituted from 1 to 3 times by substituents chosen independently from a halogen atom and an alkyl, haloalkyl or alkoxy radical,

X representing a bond or a linear or branched alkylene radical containing 1 to 5 carbon atoms,

Y representing a saturated carbon-containing cyclic system containing 1 to 3 condensed rings chosen independently from rings with 3 to 7 members, or Y representing a saturated heterocycle containing 1 to 2 heteroatoms chosen independently from O, N and S and attached to the X radical by an N or CH member, said saturated heterocycle containing moreover 2 to 6 additional members chosen independently from —CHR⁷—, —CO—, —NR⁸—, —O— and —S—, R⁷ representing a hydrogen atom or an alkyl radical and R⁸ representing a hydrogen atom or an alkyl or aralkyl radical, or also Y representing a carbocyclic or heterocyclic aryl radical optionally substituted 1 to 3 times by substituents chosen independently from the group constituted by a halogen atom, an alkyl radical, a haloalkyl radical, an alkoxy radical, a haloalkoxy radical, a hydroxy radical, a nitro radical, a cyano radical, the phenyl radical, an SO₂NHR⁹ radical and an NR¹⁰R¹¹ radical, R⁹ representing a hydrogen atom or an alkyl or phenyl radical, and R¹⁰ and R¹¹ independently representing alkyl radicals,

Z representing a bond or a linear or branched alkylene radical containing 1 to 5 carbon atoms,

R⁵ and R⁶ being chosen independently from a hydrogen atom, an alkyl, aralkyl or —(CH₂)ₙ—OH radical in which n represents an integer from 1 to 6,

or R⁵ representing an alkoxycarbonyl, haloalkoxycarbonyl or aralkoxycarbonyl radical and R⁶ representing a hydrogen atom or a methyl radical,

or also R⁵ and R⁶ forming together with the nitrogen atom a heterocycle with 4 to 7 members comprising 1 to 2 heteroatoms, the members necessary to complete the heterocycle being chosen independently from the —CR¹²R¹³, —O—, —S— and —NR¹⁴— radicals, R¹² and R¹³ representing independently each time that they occur a hydrogen atom or an alkyl radical, and R¹⁴ representing a hydrogen atom or an alkyl or aralkyl radical, or also R¹⁴ representing a phenyl radical optionally substituted 1 to 3 times by substituents chosen independently from a halogen atom and an alkyl or alkoxy radical,

R² representing a hydrogen atom or an alkyl or aralkyl radical;

or also R¹ and R² forming together with the nitrogen atom a heterocycle with 4 to 8 members comprising 1 to 2 heteroatoms, the members necessary to complete the heterocycle being chosen independently from the —CR¹⁵R¹⁶—, —O—, —S— and —NR¹⁷— radicals, R¹⁵ and R¹⁶ representing independently each time that they occur a hydrogen atom or an alkyl radical, and R¹⁷ representing a hydrogen atom or an alkyl or aralkyl radical;

R³ represents a hydrogen atom, a halogen atom, or an alkyl, haloalkyl, alkoxy or alkylthio radical;

R⁴ represents an alkyl, cycloalkyl, cycloalkylalkyl, cyano, amino, —CH₂—COOR¹⁸, —CH₂CO—NR¹⁹R²⁰ or —CH₂—NR²¹R²² radical, or R⁴ represents a carbocyclic or heterocyclic aryl radical optionally substituted 1 to 4 times by substituents chosen independently from a halogen atom and an alkyl, haloalkyl, alkoxy, haloalkoxy or NR³⁷R³⁸ radical, or also R⁴ represents a phenyl radical possessing two substituents which form together a methylenedioxy or ethylenedioxy radical,

R¹⁸ representing a hydrogen atom or an alkyl radical,

R¹⁹ representing a hydrogen atom, an alkyl radical or an aralkyl radical the aryl group of which is optionally substituted 1 to 3 times by substituents chosen independently from the group constituted by a halogen atom, an alkyl radical, a haloalkyl radical, an alkoxy radical, a haloalkoxy radical, a hydroxy radical, a nitro radical, a cyano radical, the phenyl radical, an SO₂NHR²³ radical and an NR²⁴R²⁵ radical, R²³ representing a hydrogen atom or an alkyl or phenyl radical, and R²⁴ and R²⁵ independently representing alkyl radicals,

R²⁰ representing a hydrogen atom or an alkyl radical,

or also R¹⁹ and R²⁰ forming together with the nitrogen atom a heterocycle with 4 to 7 members comprising 1 to 2 heteroatoms, the members necessary to complete the heterocycle being chosen independently from the —CR²⁶R²⁷—, —O—, —S— and —NR²⁸— radicals, R²⁶ and R²⁷ representing independently each time that they occur a hydrogen atom or an alkyl radical, and

R²⁸ representing a hydrogen atom or an alkyl or aralkyl radical, or also R²⁸ representing a phenyl radical optionally substituted 1 to 3 times by substituents chosen independently from a halogen atom and an alkyl or alkoxy radical,

R²¹ representing a hydrogen atom, an alkyl radical or an aralkyl radical the aryl group of which is optionally substituted 1 to 3 times by substituents chosen independently from the group constituted by a halogen atom, an alkyl radical, a haloalkyl radical, an alkoxy radical, a haloalkoxy radical, a hydroxy radical, a nitro radical, a cyano radical, the phenyl radical, an SO₂NHR²⁹ radical and an NR³⁰R³¹ radical, R²⁹ representing a hydrogen atom or an alkyl radical, or phenyl radical, and R³⁰ and R³¹ independently representing alkyl radicals,

R²² representing a hydrogen atom, an alkyl radical,

or also R²¹ and R²² forming together with the nitrogen atom a heterocycle with 4 to 7 members comprising 1 to 2 heteroatoms, the members necessary to complete the heterocycle being chosen independently from the —CR³²R³²—, —O—, —S— and —NR³⁴— radicals, R³³ and R³³ independently representing each time that they occur a hydrogen atom or an alkyl radical, and R³⁴ representing a hydrogen atom, an alkyl or aralkyl radical, or also R³⁴ representing a phenyl radical optionally substituted 1 to 3 times by substituents chosen independently from a halogen atom and an alkyl or alkoxy radical,

R³⁷ and R³⁸ being chosen independently from a hydrogen atom and an alkyl radical or R³⁷ and R³⁸ forming together with the nitrogen atom a heterocycle with 4 to 7 members comprising 1 to 2 heteroatoms, the members necessary to complete the heterocycle being chosen independently from the —CR³⁹R⁴⁰—, —O—, —S— and —NR⁴¹— radicals, R³⁹ and R⁴⁰ independently representing each time that they occur a hydrogen atom or an alkyl radical, and R⁴¹ representing a hydrogen atom or an alkyl radical; and

W represents O or S;

or the pharmaceutically acceptable salts of compounds of general formula (I) defined above

as Cdc25 phosphatase inhibitors, and in particular Cdc25-C phosphatase and/or of CD 45 phosphatase inhibitors. Said compounds can therefore be used for preparing a medicament intended to inhibit Cdc25 phosphatases, and in particular Cdc25-C phosphatase, and/or CD 45 phosphatase.

By alkyl, unless specified otherwise, is meant a linear or branched alkyl radical containing 1 to 12 carbon atoms, preferably 1 to 10 carbon atoms and more preferentially 1 to 8 carbon atoms (and in particular 1 to 6 carbon atoms). By lower alkyl, unless specified otherwise, is meant a linear or branched alkyl radical containing 1 to 6 carbon atoms. By cycloalkyl, unless specified otherwise, is meant a cycloalkyl radical containing 3 to 7 carbon atoms. By carbocyclic or heterocyclic aryl, is meant a carbocyclic or heterocyclic system with 1 to 3 condensed rings comprising at least one aromatic ring, a system being said to be heterocyclic when at least one of the rings which forms it comprises a heteroatom (O, N or S); when a carbocyclic or heterocyclic aryl radical is said to be substituted unless it is specified otherwise, it is meant that said carbocyclic or heterocyclic aryl radical is substituted 1 to 3 times, and preferably 1 to 2 times by radicals different to a hydrogen atom which, if they are not specified, are chosen from a halogen atom and the alkyl or alkoxy radicals; moreover, unless otherwise specified, by aryl is meant a carbocyclic aryl exclusively. By haloalkyl, is meant an alkyl radical of which at least one (and optionally all) of the hydrogen atoms is replaced by a halogen atom.

By cycloalkylalkyl, alkoxy, haloalkyl, haloalkoxy and aralkyl radicals, is meant respectively the cycloalkylalkyl, alkoxy, haloalkyl, haloalkoxy and aralkyl radicals, the alkyl, cycloalkyl and aryl radicals of which have the meanings indicated previously.

When it is indicated that a radical is optionally substituted 1 to 3 times, it is preferably optionally substituted 1 to 2 times and more preferentially optionally substituted once.

By linear or branched alkyl having 1 to 6 carbon atoms, is meant in particular the methyl, ethyl, propyl, isopropyl, butyl, isobutyl, sec-butyl and tert-butyl, pentyl, neopentyl, isopentyl, hexyl, isohexyl radicals. By haloalkyl, is meant in particular the trifluoromethyl radical. By haloalkoxy, is meant in particular the trifluoromethoxy radical. By carbocyclic aryl, is meant in particular the phenyl and naphthyl radicals. By aralkyl, is meant in particular the phenylalkyl radicals, and in particular the benzyl radical. By saturated carbon-containing cyclic system containing 1 to 3 condensed rings chosen independently from rings with 3 to 7 members, is meant in particular the cyclopropyl, cyclobutyl, cyclohexyl and adamantyl radicals. By heterocyclic aryl or heteroaryl, is meant in particular the thienyl, furannyl, pyrrolyl, imidazolyl, thiazolyl, oxazolyl and pyridyl radicals. Finally, by halogen, is meant the fluorine, chlorine, bromine or iodine atoms.

By pharmaceutically acceptable salt, is meant in particular the addition salts with inorganic acids such as hydrochloride, hydrobromide, hydroiodide, sulphate, phosphate, diphosphate and nitrate or with organic acids such as acetate, maleate, fumarate, tartrate, succinate, citrate, lactate, methanesulphonate, p-toluenesulphonate, pamoate and stearate. Also included in the scope of the present invention, when they can be used, are the salts formed from bases such as sodium or potassium hydroxide. For other examples of pharmaceutically acceptable salts, reference can be made to “Salt selection for basic drugs”, Int. J. Pharm. (1986), 33, 201-217.

In certain cases, the compounds of general formula (I) can contain asymmetrical carbon atoms. As a result, the compounds according to the present invention have two possible enantiomeric forms, i.e. the “R” and “S” configurations. The present invention includes the two enantiomeric forms and any combinations of these forms, including the racemic “RS” mixtures. For the sake of simplicity, when no specific configuration is indicated in the structural formulae, it should be understood that the two enantiomeric forms and their mixtures are represented.

The products according to the present invention comprising a compound of general formula (I) also generally present four variants:

according to a first variant, the compounds of general formula (I) which correspond also to the general sub-formula (I)₁

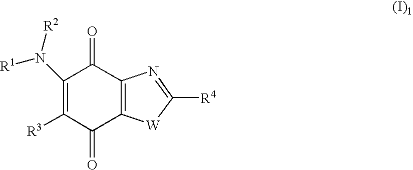

in which W represents S and R¹, R², R³ and R⁴ have the same meaning as in general formula (I), or their pharmaceutically acceptable salts, are the compounds of general formula (I) included in the product of the invention;

according to a second variant, the compounds of general formula (I) which also correspond to the general sub-formula (I)₂

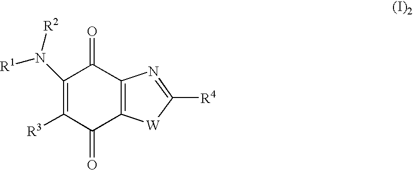

in which W represents O and R¹, R², R³ and R⁴ have the same meaning as in general formula (I), or their pharmaceutically acceptable salts, are the compounds of general formula (I) included in the product of the invention;

according to a third variant, the compounds of general formula (I) which also correspond to the general sub-formula (I)₃

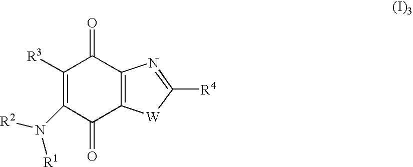

in which W represents S and R¹, R², R³ and R⁴ have the same meaning as in general formula (I), or their pharmaceutically acceptable salts, are the compounds of general formula (I) included in the product of the invention; and

according to a fourth variant, the compounds of general formula (I) which also correspond to the general sub-formula (I)₄

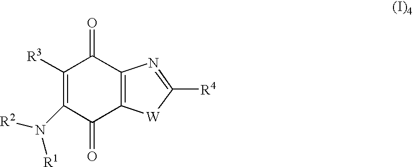

in which W represents O and R¹, R², R³ and R⁴ have the same meaning as in general formula (I), or their pharmaceutically acceptable salts, are the compounds of general formula (I) included in the product of the invention.

The invention relates in particular therefore to the products mentioned previously comprising at least one compound chosen from the compounds of general formula (I)₁ or (I)₂, or their pharmaceutically acceptable salts. Similarly, the invention relates to the products mentioned previously comprising at least one compound chosen from the compounds of general formula (I)₃ or (I)₄, or their pharmaceutically acceptable salts.

Preferably, the compounds of general formula (I), (I)₁, (I)₂, (I)₃ or (I)₄ included in a product according to the invention will have at least one of the following characteristics:
R¹ representing an alkyl, cycloalkyl, alkoxyalkyl, —(CH₂)—X—Y, —(CH₂)-Z-NR⁵R⁶ or —CHR³⁵R³⁶ radical;
R² representing a hydrogen atom or the methyl, ethyl or benzyl radical;
R¹ and R² forming together with the nitrogen atom a heterocycle with 4 to 8 members (preferably 5 to 7 members, and in particular 6 members) comprising 1 to 2 heteroatoms (and preferably 2 heteroatoms), the members necessary to complete the heterocycle being chosen independently from the —CH₂—, —O— and —NR¹⁷ radicals (and preferably from the —CH₂— and —NR¹⁷— radicals), R¹⁷ representing a methyl or benzyl radical;
R³ representing a hydrogen atom, a halogen atom or an alkyl, alkoxy or alkylthio radical;
R⁴ representing an alkyl, —CH₂—COOR¹⁸ or —CH₂—CO—NR¹⁹R²⁰ or —CH₂—NR²¹R²² radical or also a carbocyclic or heterocyclic aryl radical optionally substituted 1 to 4 times (and in particular 1 to 3 times) by substituents chosen independently from a halogen atom and an alkyl, haloalkyl, alkoxy or NR³⁷R³⁸ radical.

Generally, for a product according to the invention, the compounds of general formula (I) are preferred in which W represents a sulphur atom. Another useful alternative for a product according to the invention consists nevertheless of including the compounds of general formula (I) in which W represents an oxygen atom.

Moreover, the X radical will preferably represent a bond or a linear alkylene radical containing 1 to 5 carbon atoms. Also preferably, the Y radical will represent a saturated carbon-containing cyclic system containing 1 to 3 condensed rings chosen independently from rings with 3 to 7 members, or Y will represent a carbocyclic aryl radical optionally substituted (preferably optionally substituted by 1 to 3 radicals chosen from a halogen atom and an alkyl, haloalkyl, alkoxy, haloalkoxy, SO₂NHR⁹ or NR¹⁰R¹¹ radical, and more preferentially optionally substituted by 1 to 3 radicals chosen from a halogen atom and an alkyl, alkoxy, SO₂NHR⁹ or NR¹⁰R¹¹ radical) or also Y will represent an optionally substituted heterocyclic aryl radical, said heterocyclic aryl radical being preferably chosen from the aryl radicals with 5 members (and in particular from the imidazolyl, thienyl or pyridinyl radicals) and preferably optionally substituted by 1 to 3 radicals chosen from a halogen atom and an alkyl, haloalkyl, alkoxy, haloalkoxy, SO₂NHR⁹ or NR¹⁰R¹¹ radical, and more preferentially optionally substituted by 1 to 3 radicals chosen from a halogen atom and an alkyl, alkoxy, SO₂NHR⁹ or NR¹⁰R¹¹ radical; R⁹ will preferably represent a hydrogen atom and R¹⁰ and R¹¹ will preferably represent radicals chosen independently from the alkyl radicals. The Z radical will preferably represent an alkylene radical containing 1 to 5 carbon atoms, and in particular a —(CH₂)ₚ— radical in which p represents an integer from 1 to 3 (p preferably being equal to 1 or 2 and more preferentially equal to 1). Also preferably, R⁵ and R⁶ are chosen independently from a hydrogen atom and an alkyl radical, or also R⁵ and R⁶ form together with the nitrogen atom which carries them a heterocycle with 4 to 7 members comprising 1 to 2 heteroatoms, said heterocycle then preferably being one of the azetidinyl, pyrrolidinyl, piperidinyl, piperazinyl, homopiperazinyl, morpholinyl and thiomorpholinyl radicals optionally substituted by 1 to 3 alkyl radicals (and preferably by 1 to 3 methyl radicals); even more preferentially, R⁵ and R⁶ will be chosen independently from alkyl or alkoxycarbonyl radicals (and in particular R⁵ and R⁶ will each be a methyl or tert-butoxycarbonyl radical) or R⁵ and R⁶ form together with the nitrogen atom which carries them a heterocycle with 4 to 7 members comprising 1 to 2 heteroatoms, said heterocycle then preferably being one of the azetidinyl, pyrrolidinyl, piperidinyl, piperazinyl, homopiperazinyl, morpholinyl and thiomorpholinyl radicals optionally substituted by 1 to 3 alkyl radicals (and preferably by 1 to 3 methyl radicals). R¹⁸ will preferably represent a hydrogen atom or the methyl or ethyl radical.

Moreover, the R⁷, R¹², R¹³, R¹⁵, R¹⁶, R²⁶, R²⁷, R³⁹ and R⁴⁰ radicals are preferably chosen independently from a hydrogen atom and a methyl radical and the R⁸, R¹⁴, R¹⁷, R²⁸ and R⁴¹ radicals are preferably chosen independently from a hydrogen atom and a methyl or benzyl radical.

Moreover, as regards R¹⁹ and R²⁰, the case in which R¹⁹ represents a hydrogen atom, an alkyl radical or a benzyl radical and R²⁰ represents a hydrogen atom or the methyl radical will be preferred, as well as those in which R¹⁹ and R²⁰ form together with the nitrogen atom which carries them a heterocycle with 4 to 7 members comprising 1 to 2 heteroatoms, said heterocycle then preferably being one of the azetidinyl, pyrrolidinyl, piperidinyl, piperazinyl, homopiperazinyl, morpholinyl and thiomorpholinyl radicals optionally substituted by 1 to 3 alkyl radicals (and preferably optionally substituted by 1 to 3 methyl radicals).

Moreover, as regards R²¹ and R²², the cases in which R²¹ represents a hydrogen atom, an alkyl radical or a benzyl radical and R²² represents a hydrogen atom or the methyl radical, as well as those in which R²¹ and R²² form together with the nitrogen atom which carries them a heterocycle with 4 to 7 members comprising 1 to 2 heteroatoms, said heterocycle then preferably being one of the optionally substituted azetidinyl, pyrrolidinyl, piperidinyl, piperazinyl, homopiperazinyl, morpholinyl and thiomorpholinyl radicals are preferred. As regards the corresponding R³², R³³ and R³⁴ radicals, these are preferably such that R³² and R³³ are chosen independently from a hydrogen atom and an alkyl radical and preferably from a hydrogen atom and a methyl radical (R³² and R³³ still more preferentially both representing hydrogen atoms) and that R³⁴ represents a hydrogen atom, an alkyl radical or a phenyl radical optionally substituted 1 to 3 times by substituents chosen independently from a halogen atom and an alkyl or alkoxy radical (R³⁴ still more preferentially representing a hydrogen atom or a methyl or phenyl radical).

Moreover, as regards R³⁵ and R³⁶, the cases in which R³⁵ and R³⁶ form together with the atom of carbon which carries them an indanyl radical or R³⁵ and R³⁶ form together with the carbon atom which carries them a saturated heterocycle containing 5 to 7 members and 1 to 2 heteroatoms chosen from O, N and S, the nitrogen atoms of said heterocycle being optionally substituted by radicals chosen from the alkyl radicals and the benzyl radical are preferred.

Moreover, as regards R³⁷ and R³⁸, the cases in which R³⁷ and R³⁸ independently represent radicals chosen from the alkyl radicals are preferred.

Finally, when R⁴ is a carbocyclic or heterocyclic aryl radical optionally substituted 1 to 4 times, it is preferred that it is chosen from the group consisting of carbocyclic and heterocyclic aryl radicals optionally substituted 1 to 3 times by substituents chosen independently from a halogen atom and an alkyl, haloalkyl, alkoxy, haloalkoxy or NR³⁷R³⁸ radical (and in particular 1 to 3 times by substituents chosen independently from a halogen atom and an alkyl, haloalkyl, alkoxy or haloalkoxy radical) and the 2,3,4,5-tetrafluorophenyl radical. More preferentially, when R⁴ is a carbocyclic or heterocyclic aryl radical optionally substituted 1 to 4 times, R⁴ is chosen from the group consisting of carbocyclic and heterocyclic aryl radicals optionally substituted 1 to 2 times by substituents chosen independently from a halogen atom, an alkyl, haloalkyl, alkoxy, haloalkoxy or NR³⁷R³⁸ radical (and in particular 1 to 2 times by substituents chosen independently from a halogen atom and an alkyl, haloalkyl, alkoxy or haloalkoxy radical), a 3,4,5-trihalophenyl radical and the 2,3,4,5-tetrafluorophenyl radical.

More preferentially, the compounds of general formula (I), (I)₁, (I)₂, (I)₃ or (I)₄ included in a product according to the invention will have at least one of the following characteristics:
R¹ representing an alkyl, cycloalkyl, or —(CH₂)-Z-NR⁵R⁶ radical;
R² representing a hydrogen atom or the methyl radical;
R³ representing a hydrogen atom, a halogen atom or the methoxy radical;
R⁴ representing an alkyl, —CH₂—NR²¹R²² radical, or also a carbocyclic or heterocyclic aryl radical optionally substituted 1 to 4 times (and in particular 1 to 3 times) by substituents chosen independently from a halogen atom and an alkyl, or NR³⁷R³⁸ radical.

Still more preferentially, the compounds of general formula (I), (I)₁, (I)₂, (I)₃ or (I)₄ included in a product according to the invention will have at least one of the following characteristics:
R¹ representing a —(CH₂)-Z-NR⁵R⁶ radical;
R² representing a hydrogen atom;
R³ representing a hydrogen atom or a halogen atom (said halogen atom being preferably a chlorine or bromine atom);
R⁴ representing an alkyl radical or also a phenyl, pyridyl, thienyl or furannyl radical optionally substituted by 1 to 4 (preferably 1 to 3) halogen atoms or by an NR³⁷R³⁸ radical.

Still more preferentially, the compounds of general formula (I), (I)₁, (I)₂, (I)₃ or (I)₄ included in a product according to the invention will have at least one of the following characteristics:
R³ representing a hydrogen atom or a chlorine atom (and more preferentially a hydrogen atom);
R⁴ representing an alkyl radical or also a phenyl, pyridyl, thienyl furannyl radical optionally substituted by 1 to 4 (preferably 1 to 3) halogen atoms (and in particular R⁴ representing an alkyl radical, and preferably an alkyl radical containing 1 to 4 carbon atoms, and still more preferentially a methyl or ethyl radical).

According to a particular variant of the invention, W represents O. In this particular case, it is preferred that R¹ represents an aryl radical, and in particular a phenyl radical, optionally substituted 1 to 3 times by substituents chosen independently from a halogen atom and an alkyl, haloalkyl or alkoxy radical. More preferentially, still when W represents O, it will be preferred that R¹ represents a phenyl radical optionally substituted by a halogen atom (said halogen atom being preferably a fluorine atom).

According to a particular aspect of the invention, R⁴ will represent a phenyl radical or a heterocyclic aryl radical with 5 to 6 members optionally substituted 1 to 4 times (and preferably 1 to 3 times) by substituents chosen from the group consisting of halogen atoms, the trifluoromethyl radical and the trifluoromethoxy radical (and preferably chosen from the group consisting of halogen atoms and the trifluoromethyl radical). In particular, said optionally substituted heterocyclic aryl with 5 to 6 members is an optionally substituted pyridine, thiophene, furane or pyrrole ring.

Another particular aspect of this invention relates to the use of compounds of general formula (I) in which W represents S, R³ represents a hydrogen atom, the —NR¹R² substituent (the preferences indicated previously for R¹ and R² are still applicable) is attached at position 5 of the benzothiazoledione ring and R⁴ is chosen from the alkyl, cycloalkylalkyl, —CH₂—COOR¹⁸, —CH₂—CO—NR¹⁹R²¹ and —CH₂—NR²¹R²² radicals (R⁴ being preferably alkyl or cycloalkylalkyl and more preferentially alkyl according to this particular aspect of the invention).

For a product according to the invention, the compounds of general formula (I) described (if appropriate in the form of salts or mixtures) in Examples 1 to 138 of compounds of general formula (I), or the pharmaceutically acceptable salts of such compounds, are particularly preferred. Among the compounds of Examples 1 to 138 of compounds of general formula (I) and their pharmaceutically acceptable salts, the compounds of Examples 1 to 14, 18 to 39, 48 to 52, 55, 57, 58 and 60 to 138 will generally be of more use for including in a product according to this invention.

Moreover, the compounds of general formula (I) described (if appropriate in the form of salts or mixtures) in Examples 2 to 5, 16, 19 to 26, 32, 34, 38 to 40, 43 to 47, 55 to 58, 60 to 77, 79 to 98 and 101 to 115 of compounds of general formula (I), or the pharmaceutically acceptable salts of such compounds, are still more particularly preferred for including in a product according to the invention.

Moreover, the compounds of general formula (I) described (if appropriate in the form of salts or mixtures) in Examples 2, 19, 20, 23, 24, 34, 57, 60, 62, 63, 67 to 77, 80 to 92, 94, 96 to 98, 103, 104, 106 and 110 to 113 of compounds of general formula (I), or the pharmaceutically acceptable salts of such compounds, are most particularly preferred for including in a product according to the invention:

Particularly preferably, the products according to the invention comprising a compound of general formula (I) will include a compound chosen from the following compounds:
5-{[2-(dimethylamino)ethyl]amino}-2-methyl-1,3-benzothiazole-4,7-dione;
2-methyl-5-[(2-pyrrolidin-1-ylethyl)amino]-1,3-benzothiazole-4,7-dione;
2-methyl-5-[(2-piperidin-1-ylethyl)amino]-1,3-benzothiazole-4,7-dione;
2-(2-chloro-6-fluorophenyl)-5-{[2-(dimethylamino)ethyl]amino}-1,3-benzothiazole 4,7-dione;

and the pharmaceutically acceptable salts of the latter.

According to a still more preferred aspect of the invention, the products according to the invention comprising a compound of general formula (I) will include 5-{[2-(dimethylamino)ethyl]amino}-2-methyl-1,3-benzothiazole-4,7-dione or one of its pharmaceutically acceptable salts.

Alternatively, the Cdc25 phosphatase inhibitor can be a compound of general formula (II)

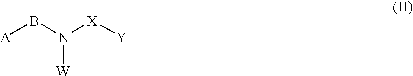

in which:

A represents an (AI) radical

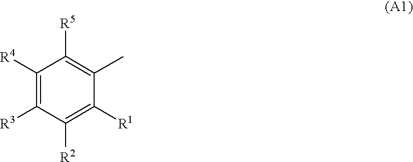

in which two of the R¹, R², R³, R⁴ and R⁵ groups represent hydrogen atoms and the three others are chosen independently from a hydrogen atom, a halogen atom and an alkyl, hydroxy, alkoxy, alkylcarbonyloxy, alkylthio or NR⁶R⁷ radical, it being understood moreover that:

either R¹ and one of R² and R⁴ are chosen independently from a hydroxy, alkylcarbonyloxy and NR⁶R⁷ radical,

or R² and one of R³ and R⁵ are chosen independently from a hydroxy, alkylcarbonyloxy and NR⁶R⁷ radical,

or R⁴ and one of R³ and R⁵ are chosen independently from a hydroxy, alkylcarbonyloxy and NR⁶R⁷ radical,

or also one of R¹, R³ and R⁵ is chosen from a hydroxy, alkylcarbonyloxy and NR⁶R⁷ radical, and the remainder B—N(W)—X—Y is attached to the A radical by a nitrogen atom, R⁶ and R⁷ representing, independently each time that they occur, a hydrogen atom or an alkyl radical or R⁶ and R⁷ forming together with the nitrogen atom a heterocycle with 4 to 7 members comprising 1 to 2 heteroatoms, the members necessary to complete the heterocycle being chosen independently from the —CR⁸R⁹—, —O—, —S— and —NR¹⁰— radicals, R⁸ and R⁹ representing independently each time that they occur a hydrogen atom or an alkyl, alkoxy, benzyloxycarbonylamino or dialkylamino radical, and R¹⁰ representing independently each time that it occurs a hydrogen atom or an alkyl radical,

or also A represents an (A2) radical

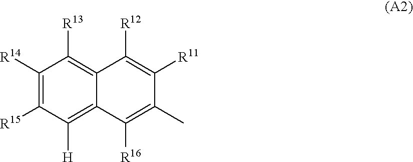

in which:

either R¹¹ and one of R¹³, R¹⁴ and R¹⁵ represent hydroxy radicals while the other radicals from R¹³, R¹⁴ and R¹⁵ as well as R¹⁶ represent hydrogen atoms,

or R¹² and R¹⁶ represent hydroxy radicals while R¹¹, R¹³, R¹⁴ and R¹⁵ represent hydrogen atoms;

B represents a —CO—, —NH—CO—(CH₂)ₙ— or —(CH₂)ₚ— radical, n being an integer from 0 to 3 and p being an integer from 0 to 1;

W represents a hydrogen atom or an alkyl radical;

X represents a —(CH₂)_{q}—, —(CH₂)_{q}—NH— or —CO—(CH₂)ᵣ— radical, q being an integer from 1 to 6 and r an integer from 0 to 6;

or also the B—N(W)—X—Y group is such that it represents the radical

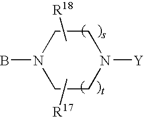

in which B is as defined above, t is an integer from 0 to 2, s is an integer from 0 to 1 and R¹⁷ and R¹⁸ represent radicals chosen independently from a hydrogen atom and an alkyl radical;

and:

when X represents a —(CH₂)_{q}— or —CO—(CH₂)ᵣ— radical, then Y represents a radical

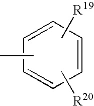

in which R¹⁹ represents a hydrogen atom, a halogen atom, a nitro, alkyl, alkylthio, NR²¹R²², —SO₂—NR²³R²⁴, —NH—SO₂—R²⁵ or —O—P(O)(OR²⁶)(OR²⁷) radical,

R²¹ and R²² independently representing a hydrogen atom, or an alkyl radical,

R²³ and R²⁴ independently representing a hydrogen atom, or an alkyl radical, or R²³ and R²⁴ representing together with the nitrogen atom which carries them a heterocycle with 5 to 7 members the complementary members of which are chosen independently from —CHR²⁸—, —NR²⁹—, —O— and —S—, R²⁸ representing, independently each time that they occur, a hydrogen atom or an alkyl radical,

R²⁵ representing an alkyl, haloalkyl radical or one of the aryl, heteroaryl, aralkyl or heteroaralkyl radicals the aryl or heteroaryl nucleus of which is optionally substituted by one or more radicals chosen independently from a halogen atom and alkyl, haloalkyl, hydroxy, alkoxy or nitro radicals, except for the optional nitrogen atoms of the heteroaryl nucleus the optional substituents of which are chosen from the alkyl radicals, R²⁶ and R²⁷ being chosen independently from alkyl radicals,

and R²⁰ represents a hydrogen atom, a halogen atom or an alkyl, alkoxy or alkylthio radical,

or also Y represents the (T) radical represented below

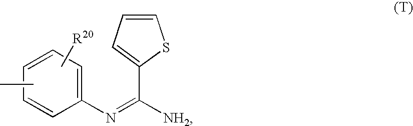

in which R²⁰ represents a hydrogen atom or an alkyl, alkoxy or alkylthio radical,

when X represents a —(CH₂)_{q}—NH— radical or when the B—N(W)—X—Y group is such that it represents the radical

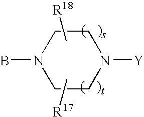

then Y represents exclusively an —SO₂—R³⁰ radical in which R³⁰ represents an alkyl, haloalkyl radical or one of the aryl, heteroaryl, aralkyl or heteroaralkyl radicals the aryl or heteroaryl nucleus of which is optionally substituted by one or more radicals chosen independently from a halogen atom and the alkyl, haloalkyl, hydroxy, alkoxy or nitro radicals, except for the optional nitrogen atoms of the heteroaryl nucleus the optional substituents of which are chosen from the alkyl radicals;

it being understood moreover that when the B—N(W)—X—Y group is such that it represents the radical

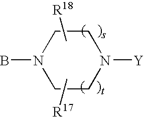

then B represents exclusively a —CO— or —(CH₂)— radical;

or a pharmaceutically acceptable salt of such a compound.

Preferably, the compounds of general formula (II) are chosen from the following compounds:
4-(dimethylamino)-2-methoxy-6-({methyl[2-(4-nitrophenyl)ethyl]amino}methyl)-phenol;
4-(dimethylamino)-2-({methyl[2-(4-nitrophenyl)ethyl]amino}methyl)phenol;
2,7-dihydroxy-N-{2-[4-[(2-thienyl(imino)methyl)amino]phenyl]ethyl}-2-napthalenecarboxamide;
3-[(3-{[amino(2-thienyl)methylidene]amino}-benzyl)amino]-N-[4-(dimethylamino)phenyl]propanamide;
4-(4-aminophenyl)-N-[4-(4-methyl-1-piperazinyl)phenyl]butanamide;
4-(dimethylamino)-2-methoxy-6-({[2-(4-nitrophenyl)ethyl]amino}methyl)phenol;
4-(dimethylamino)-2-({[2-(4-nitrophenyl)ethyl]amino}methyl)phenol;
2-(dimethylamino)-6-methoxy-4-({methyl[2-(4-nitrophenyl)ethyl]amino}methyl)phenol;
2-({methyl[2-(4-nitrophenyl)ethyl]amino}methyl)-1,4-benzenediol;
4-(dimethylamino)-2-methoxy-6-({methyl[2-(4-nitrophenyl)ethyl]amino}methyl)phenyl acetate;
3,7-dihydroxy-N-[2-(4-nitrophenyl)ethyl]-2-naphthamide;
N-[4-(dimethylamino)benzyl]-3,7-dihydroxy-2-naphthamide;
diethyl 4-{2-[(3,7-dihydroxy-2-naphthoyl)amino]ethyl}phenylphosphate;
N-{2-[4-(aminosulphonyl)phenyl]ethyl}-3,7-dihydroxy-2-naphthamide;
3,7-dihydroxy-N-[2-(4-aminophenyl)ethyl]-2-naphthamide;
3,7-dihydroxy-N-(2-{4-[(methylsulphonyl)amino]phenyl}ethyl)-2-naphthamide;
N-(2-{4-[(butylsulphonyl)amino]phenyl}ethyl)-3,7-dihydroxy-2-naphthamide;
3,7-dihydroxy-N-[2-(4-{[(4-methylphenyl)sulphonyl]amino}phenyl)ethyl]-2-naphthamide;
3,7-dihydroxy-N-(2-{4-[(1-naphthylsulphonyl)amino]phenyl}ethyl)-2-naphthamide;
3,7-dihydroxy-N-{2-[4-({[2-(trifluoromethyl)phenyl]sulphonyl}amino)phenyl]ethyl}-2-naphthamide;
N-(2-{4-[(benzylsulphonyl)amino]phenyl}ethyl)-3,7-dihydroxy-2-naphthamide;
3,7-dihydroxy-N-{2-[4-({[3-(trifluoromethyl)phenyl]sulphonyl}amino)phenyl]ethyl}-2-naphthamide;
3,7-dihydroxy-N-[2-(4-{[(4-nitrophenyl)sulphonyl]amino}phenyl)ethyl]-2-naphthamide;
3,7-dihydroxy-N-{2-[4-({[4-(trifluoromethyl)phenyl]sulphonyl}amino) phenyl]ethyl}-2-naphthamide;
3,7-dihydroxy-N-(2-{4-[(thien-2-ylsulphonyl)amino]phenyl}ethyl)-2-naphthamide;
3,7-dihydroxy-N-[2-(4-{[(4-methoxyphenyl)sulphonyl]amino}phenyl)ethyl]-2-naphthamide;
3,7-dihydroxy-N-[2-(4-{[(1-methyl-1H-imidazol-4-yl)sulphonyl]amino}phenyl)ethyl]-2-naphthamide;
N-[2-(4-{[(4-fluorophenyl)sulphonyl]amino}phenyl)ethyl]-3,7-dihydroxy-2-naphthamide;
3,7-dihydroxy-N-{3-[(4-methyl-1-piperidinyl)sulphonyl]benzyl}-2-naphthamide;
5-(4-{[1E)-amino(2-thienyl)methylidene]amino}phenyl)-N-[2-(dimethylamino)phenyl]pentanamide;
3-({4-[(4-methylphenyl)sulphonyl]piperazin-1-yl}carbonyl)naphthalene-2,6-diol;
3-{[4-(methylsulphonyl)piperazin-1-yl]carbonyl}naphthalene-2,6-diol;
3-{[4-(butylsulphonyl)piperazin-1-yl]carbonyl}naphthalene-2,6-diol;
   and the pharmaceutically acceptable salts of the latter.
   The Cdc25 phosphatase inhibitor included in a product according to the invention can moreover also be menadione (also known as vitamin K3) or one of its analogues such as for example 2-(2-mercaptoethanol)-3-methyl-1,4-naphthoquinone (described in Markovits et al., Life Sci. (2003), 72(24), 2769-84).

The anti-cancer agent associated with the Cdc25 phosphatase inhibitor can be chosen from anti-cancer agents as varied as:

analogues of DNA bases such as 5-fluorouracil;

Type I and/or II topoisomerase inhibitors such as for example camptothecin and its analogues, doxorubicin or amsacrine;

compounds interacting with the spindle cell such as for example paclitaxel (Taxol®) or docetaxel (Taxotere®);

compounds acting on the cytoskeleton such as vinblastine;

inhibitors of the transduction of the signal passing through the heterotrimeric G proteins;

prenyltransferase inhibitors, and in particular farnesyltransferase inhibitors;

cyclin-dependent kinase (CDKs) inhibitors;

alkylating agents such as cisplatin;

antagonists of folic acid such as methotrexate; or

inhibitors of the synthesis of DNA and cell division such as mitomycin C.

As regards the analogues of camptothecin being able to be combined with the inhibitor of Cdc25 phosphatases, these being able to be analogues comprising a lactonic E ring with six members (such as for example the compounds described in PCT Patent Application WO 94/11376), analogues comprising a lactonic E ring with seven members (such as for example homocamptothecins—the compounds described in PCT Patent Application WO 97/00876) or open tetracyclic analogues (such as for example the compounds described in PCT Patent Application WO 99/33829). Preferably, the analogue of camptothecin is chosen from the group comprising diflomotecan, (+)-9-chloro-5-ethyl-5-hydroxy-10-methyl-12-(4-methylpiperidinomethyl)-4,5,13,15-tetrahydro-1H,3H-oxepino[3′,4′:6.7]indolizino[1,2-c]quinoline-3,15-dione and its salts (in particular its hydrochloride also known under the name BN-80927) as well as the compound known under the code name SN-38.

By homocamptothecin, is meant in the present Application any analogue of camptothecin in which the pentacyclic pattern of the natural camptothecin has been modified by replacement, in the E ring, of the natural α-hydroxylactone of the camptothecin by an hydroxylactone.

According to a particular variant of the invention, the analogues of camptothecin combined with the inhibitor of Cdc25 phosphatases are analogues comprising a lactonic E ring with seven members. These are preferably homocamptothecins, and in particular homocamptothecins chosen from the compounds of general formula (III)

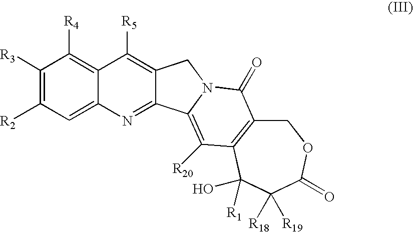

in racemic, enantiomeric form or all combinations of these forms, in which
R₁ represents a lower alkyl, a lower alkenyl, a lower alkynyl, a lower haloalkyl, a lower alkoxy lower alkyl or a lower alkylthio lower alkyl;
R₂, R₃ and R₄ represent, independently, i) H, halo, lower halo alkyl, lower alkyl, lower alkenyl, cyano, lower cyano alkyl, nitro, lower nitro alkyl, amido, lower amido alkyl, hydrazino, lower hydrazino alkyl, azido, lower azido alkyl, (CH₂)ₘNR₆R₇, (CH₂)ₘOR₆, (CH₂)ₘSR₆, (CH₂)ₘCO₂R₆, (CH₂)ₘNR₆C(O)R₈, (CH₂)ₘC(O)R₈, (CH₂)ₘOC(O)₂R₈, O(CH₂)ₘNR₆R₇, OC(O)NR₆R₇, OC(O)(CH₂)ₘCO₂R₆, or ii) the following radicals substituted (i.e., substituted one to four times on the aryl group or the heterocycle) or not substituted: (CH₂)ₙ[N═X], OC(O)[N═X], (CH₂)ₘOC(O)[N═X] (in which [N═X], in this invention, represents a heterocyclic group with 4 to 7 members with the nitrogen atom N, which is a member of the heterocyclic group, and X represents the remaining members, necessary to complete the heterocyclic group, selected from the group constituted by O, S, CH₂, CH, N, NR₉ and COR₁₀), aryl or lower aryl alkyl, in which the optional substituents are chosen from the group constituted by a lower alkyl, halo, nitro, amino, lower alkylamino, lower haloalkyl, lower hydroxy alkyl, lower alkoxy, and lower alkoxy lower alkyl; or R₂ and R₃ form together a chain with 3 or 4 members, in which the elements of the chain are selected from the group constituted by CH, CH₂, O, S, N or NR₉;
R₅ represents i) H, halo, lower halo alkyl, lower alkyl, lower alkoxy, lower alkoxy lower alkyl, lower alkylthio lower alkyl, cycloalkyl, lower cycloalkyl alkyl, cyano, cyano alkyl, lower alkyl lower sulphonyl alkyl, lower hydroxy alkyl, nitro, (CH₂)ₘC(O)R₈, (CH₂)ₘNR₆C(O)R₈, (CH₂)ₘNR₆R₇, (CH₂)ₘN(CH₃)(CH₂)ₙNR₆R₇, (CH₂)ₘOC(O)R₈, (CH₂)ₘOC(O)NR₆R₇, (CH₂)ₙS(O)_{q}R₁₁, (CH₂)ₘP(O)R₁₂R₁₃, (CH₂)₂P(S)R₁₂R₁₃, or ii) one of the following radicals substituted (i.e. one to four times on the aryl or heteroaryl group) or not substituted: (CH₂)ₙ[N═X], OC(O)[N═X], (CH₂)ₘOC(O)[N═X], aryl or lower aryl alkyl, in which the optional substituents are chosen from the group constituted by a lower alkyl, halo, nitro, amino, lower alkyl amino, lower halo alkyl, lower hydroxy alkyl, lower alkoxy and lower alkoxy lower alkyl;
R₆ and R₇ represent, independently, i) H, a lower alkyl, lower hydroxy alkyl, lower alkyl lower amino alkyl, lower amino alkyl, cycloalkyl, lower cycloalkyl alkyl, lower alkenyl, lower alkoxy lower alkyl, lower halo alkyl, or ii) one of the following radicals substituted (i.e., one to four times on the aryl group) or not substituted: aryl or lower aryl alkyl, in which the optional substituents are chosen from the group constituted by a lower alkyl, halo, nitro, amino, lower alkyl amino, lower halo alkyl, lower hydroxy alkyl, lower alkoxy, and lower alkoxy lower alkyl;
R₈ represents i) H, a lower alkyl, lower hydroxy alkyl, amino, lower alkyl amino, lower alkyl amino lower alkyl, lower amino alkyl, cycloalkyl, lower cycloalkyl alkyl, lower alkenyl, lower alkoxy, lower alkoxy lower alkyl, lower halo alkyl, or ii) one of the following radicals substituted (i.e., one to four times on the aryl group) or not substituted: aryl or lower aryl alkyl, in which the optional substituents are chosen from the group constituted by a lower alkyl, halo, nitro, amino, lower alkyl amino, lower halo alkyl, lower hydroxy alkyl, lower alkoxy, or lower alkoxy lower alkyl;
R₉ represents H, a lower alkyl, lower halo alkyl, aryl, or aryl substituted by one or more groups chosen from the lower alkyl, halo, nitro, amino, lower alkyl amino, lower halo alkyl, lower hydroxy alkyl, lower alkoxy, or lower alkoxy lower alkyl radical;
R₁₀ represents H, a lower alkyl, lower halo alkyl, lower alkoxy, aryl, or aryl substituted (i.e., presenting one to four substituents on the aryl group) by one or more groups chosen from the lower alkyl, lower halo alkyl, lower hydroxy alkyl, or lower alkoxy lower alkyl radical;
R₁₁ represents a lower alkyl, aryl, (CH₂)ₘOR₁₄, (CH₂)ₘSR₁₄, (CH₂)₂NR₁₄R₁₅ or (CH₂)ₘ[N═X];
R₁₂ and R₁₃ represent, independently, a lower alkyl, aryl, lower alkoxy, aryloxy or amino;
R₁₄ and R₁₅ represent, independently, H, a lower alkyl or aryl;
R₁₈ and R₁₉ represent, independently, H, halo, lower alkyl, lower alkoxy or hydroxy;
R₂₀ represents H or halo;
m is a whole number comprised between 0 and 6;
n is 1 or 2; and
q represents a whole number from 0 to 2; and [N═X] represents a heterocyclic group with 4 to 7 members, X representing the chain necessary to complete said heterocyclic group and selected from the group constituted by O, S, CH₂, CH, N, NR₉ and COR₁₀;
and the pharmaceutically acceptable salts of the latter.

Preferably the compound of general formula (III) is such that R₂ represents H or halo; R₃ represents H, a lower alkyl or halo; R₄ represents H or halo; R₅ represents H, a lower alkyl or a (CH₂)[N═X] group substituted or not substituted in which the optional substituent is a lower alkyl; or a pharmaceutically acceptable salt of the latter.

The compounds of general formula (III) or their pharmaceutically acceptable salts are more particularly chosen from diflomotecan and (+)-9-chloro-5-ethyl-5-hydroxy-10-methyl-12-(4-methylpiperidinomethyl)-4,5,13,15-tetrahydro-1H,3H-oxepino[3′,4′:6.7]indolizino[1,2-c]quinoline-3,15-dione and its pharmaceutically acceptable salts (in particular its hydrochloride also known under the name BN-80927).

As regards the inhibitors of the transduction of the signal passing through the heterotrimeric G proteins being able to be combined with the inhibitor of Cdc25 phosphatases, these can be compounds of general formula (IV)

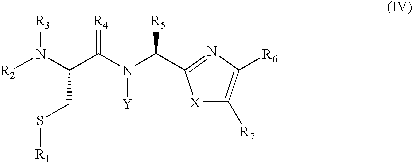

corresponding to the sub-formulae (IV_{A}) or (IV_{B}):

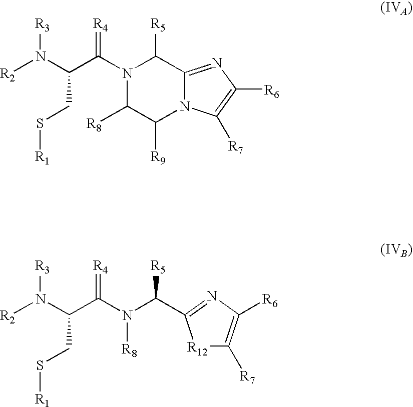

in which:

X represents R₁₂ and Y represents R₈, or X and Y complete a ring with 6 members, the X—Y group representing the —CH(R₈)—CH(R₉)— radical;

R₁ represents H, an alkyl, alkylthio or cycloalkylthio radical;

R₂ and R₃ independently represent H or an alkyl or cycloalkyl radical;

R₄ represents H₂ or O;

R₅ represents H, or one of the alkyl, alkenyl, alkynyl, cycloalkyl, cycloalkylalkyl, cycloalkenyl, cycloalkenylalkyl, aryl, aralkyl, heterocyclyl or heterocyclylalkyl radicals, these radicals being optionally substituted by radicals chosen from the group comprising an alkyl, —O—R₁₀, —S(O)ₘR₁₀ (m representing 0, 1, or 2), —N(R₁₀)(R₁₁), —N—C(O)—R₁₀, —NH—(SO₂)—R₁₀, —CO₂—R₁₀, —C(O)—N(R₁₀)(R₁₁), and —(SO₂)—N(R₁₀)(R₁₁) radical;

R₆ and R₇ independently represent H, a —C(O)—NH—CHR₁₃—CO₂R₁₄ radical, or one of the alkyl, cycloalkyl, cycloalkylalkyl, cycloalkenyl, cycloalkenylalkyl, aryl, aralkyl, heterocyclyl or heterocyclylalkyl radicals, these radicals being optionally substituted by radicals chosen from the group comprising the OH, alkyl or alkoxy, N(R₁₀)(R₁₁), COOH, CON(R₁₀)(R₁₁), and halo radicals,

or R₆ and R₇ form together an aryl radical or a heterocycle;

R₈ and R₉ independently represent, H, or one of the alkyl, cycloalkyl, cycloalkylalkyl, cycloalkenyl, cycloalkenylalkyl, aryl, aralkyl, heterocyclyl or heterocyclylalkyl radicals, these radicals being optionally substituted by radicals chosen from the group comprising the OH, alkyl or alkoxy, N(R₁₀)(R₁₁), COOH, CON(R₁₀)(R₁₁) and halo radicals, or R₈ and R₉ form together an aryl radical or a heterocycle;

R₁₀ and R₁₁, independently represent H, an aryl radical or heterocyclyl, or an alkyl, aryl, aralkyl, cycloalkyl, cycloalkylalkyl, heterocyclyl or heterocyclylalkyl radical;

R₁₂ represents NR₉, S, or O;

R₁₃ represents an alkyl radical optionally substituted by a radical chosen from the alkyl, —OR₁₀, —S(O)ₘR₁₀ (m representing 0, 1, or 2) and —N(R₁₀)(R₁₁) radicals;

R₁₄ represents H or an alkyl radical;

or pharmaceutically acceptable salts of the latter.

Among the compounds of general formula (IV) and the pharmaceutically acceptable salts of such compounds, in particular a compound chosen from 7-(2-amino-1-oxo-3-thiopropyl)-8-(cyclohexylmethyl)-2-phenyl-5,6,7,8-tetrahydroimidazo[1,2a]pyrazine and its dimer form, bis-1,1′-{7-(2-amino-1-oxo-3-thiopropyl)-8-(cyclohexylmethyl)-2-phenyl-5,6,7,8-tetrahydroimidazo[1,2a]pyrazine}disulphide or (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine, or a pharmaceutically acceptable salt of one of these compounds will be preferred.

As regards the farnesyltransferase inhibitors, these can in particular be chosen from the group composed:

of a compound of general formula (V)

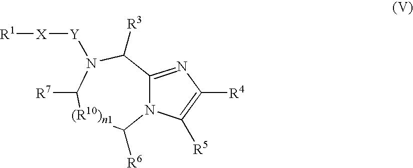

in which:

n1 represents 0 or 1;

X represents, independently each time that it occurs, (CHR¹¹)ₙ₃(CH₂)ₙ₄Z(CH₂)ₙ₅;

Z representing O, N(R¹²), S, or a bond;

n3 representing, independently each time that it occurs, 0 or 1; each of n4 and n5 representing, independently each time that they occur, 0, 1, 2, or 3;

Y represents, independently each time that it occurs, CO, CH₂, CS, or a bond;

R¹ represents one of the radicals

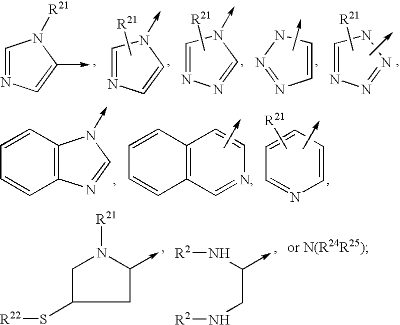

each of R², R¹¹, and R¹² representing, independently each time that it occurs, H or a optionally substituted radical chosen from the group consisting of a (C₁₋₆)alkyl radical and an aryl radical, said optionally substituted radical being optionally substituted by at least one radical chosen from the R⁸ and R³⁰ radicals, each substituent being chosen independently of the others;

R³ represents, independently each time that it occurs, H or an optionally substituted radical chosen from the group consisting of the (C₁₋₆)alkyl, (C₂₋₆)alkenyl, (C₂₋₆)alkynyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl, and heterocyclyl(C₁₋₆)alkyl radicals, said optionally substituted radical being optionally substituted by at least one radical chosen from the R³⁰ radicals, each substituent being chosen independently of the others;

each of R⁴ and R⁵ represents, independently each time that it occurs, H or an optionally substituted radical chosen from the group consisting of the (C₁₋₆)alkyl, (C₃₋₆)cycloalkyl, aryl and heterocyclyl radicals, said optionally substituted radical being optionally substituted by at least one radical chosen from the R³⁰ radicals, each substituent being chosen independently of the other, or R⁴ and R⁵ taken together with the carbon atoms to which they are attached together form an aryl radical;

R⁶ represents, independently each time that it occurs, H or an optionally substituted radical chosen from the group consisting of the (C₁₋₆)alkyl, (C₂₋₆)alkenyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl and heterocyclyl(C₁₋₆)alkyl radicals, said optionally substituted radical being optionally substituted by at least one radical chosen from the OH, (C₁₋₆)alkyl, (C₁₋₆)alkoxy, —N(R⁸R⁹), —COOH, —CON(R⁸R⁹) and halo radicals, each substituent being chosen independently of the others;

R⁷ represents, independently each time that it occurs, H, ═O, ═S, H or an optionally substituted radical chosen from the group consisting of the (C₁₋₆)alkyl, (C₂₋₆)alkenyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl and heterocyclyl(C₁₋₆)alkyl radicals, said optionally substituted radical being optionally substituted by at least one radical chosen from the OH, (C₁₋₆)alkyl, (C₁₋₆)alkoxy, —N(R⁸R⁹), —COOH, —CON(R⁸R⁹) and halo radicals, each substituent being chosen independently of the others;

each of R⁸ and R⁹ representing, independently each time that it occurs, H, (C₁₋₆)alkyl, (C₂₋₆)alkenyl, (C₂₋₆)alkynyl, aryl, or aryl(C₁₋₆)alkyl;

R¹⁰ represents C;

or, when n1=0, R⁶ and R⁷ can be taken together with the carbon atoms to which they are attached to form an aryl or cyclohexyl radical;

R²¹ represents, independently each time that it occurs, H or an optionally substituted radical chosen from the group consisting of the (C₁₋₆)alkyl and aryl(C₁₋₆)alkyl radicals, said optionally substituted radical being optionally substituted by at least one radical chosen from the R⁸ and R³⁰ radicals, each substituent being chosen independently of the others;

R²² represents H, (C₁₋₆)alkylthio, (C₃₋₆)cycloalkylthio, R⁸—CO—, or a substituent of formula

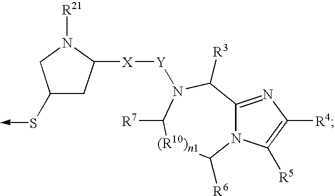

each of R²⁴ and R²⁵ represents, independently each time that it occurs, H, (C₁₋₆)alkyl or aryl(C₁₋₆)alkyl;

R³⁰ represents, independently each time that it occurs, (C₁₋₆)alkyl, —O—R⁸, —S(O)ₙ₆R⁸, —S(O)ₙ₇N(R⁸R⁹), —N(R⁸R⁹), —CN, —NO₂, —CO₂R⁸, —CON(R⁸R⁹), —NCO—R⁸, or halogen, each of n6 and n7 representing, independently each time that it occurs, 0, 1 or 2; said heterocyclyl radical being azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothio-pyranyl sulphone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl-N-oxide, quinoxalinyl, tetrahydrofuryl, tetrahydroisoquinolinyl, tetrahydroquinolinyl, thiamorpholinyl, thiamorpholinyl sulphoxide, thiazolyl, thiazolinyl, thienofuryl, thienothienyl or thienyl;

said aryl radical being phenyl or naphthyl;

it being understood that:

when n1=1, R¹⁰ is C and R⁶ represents, H, then R¹⁰ and R⁷ can form, taken together, the radical

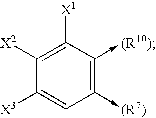

or when n1=1, R¹⁰ is C, and R⁷ is ═O, —H, or ═S, then R¹⁰ and R⁶ can form, taken together; the radical

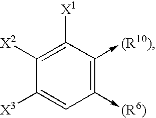

with each of X¹, X², and X³ representing, independently, H, a halogen atom, —N0₂, —NCO—R⁸, —CO₂R⁸, —CN, or —CON(R⁸R⁹); and

when R¹ is N(R²⁴R²⁵), then n3 represents 1, each of n4 and n5 represents 0, Z is a bond, and R³ and R¹¹ can form, taken together, the radical

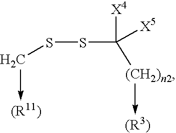

with n2 representing an integer from 1 to 6, and each of X⁴ and X⁵ representing, independently, H, (C₁₋₆)alkyl or aryl, or X⁴ and X⁵ forming, taken together, a (C₃₋₆)cycloalkyl radical;

of a compound of general formula (VI)

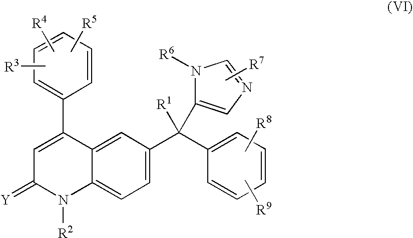

in which:

R¹ represents H or an alkyl radical, OR¹⁰, SR¹⁰ or NR¹¹R¹²

R² represents H or an alkyl radical;

R³, R⁴ and R⁵ represent, independently, H, a halogen atom or an alkyl, trihalomethyl, hydroxy, cyano or alkoxy radical;

R⁶ represents H or an alkyl radical;

R⁷ represents H, a halogen atom or an alkyl, hydroxyalkyl, amino, hydroxycarbonyl radical;

R⁸ and R⁹ represent, independently, H, a halogen atom or a cyano, alkyl, trihalomethyl, alkoxy, alkylthio or dialkylamino radical;

R¹⁰ represents H or an alkyl or alkylcarbonyl radical;

R¹¹ represents H or an alkyl radical;

R¹² represents H or an alkyl or alkylcarbonyl radical;

and Y represents O or S;
and a pharmaceutically acceptable salt of a compound of general formula (V) or of a compound of general formula (VI).

When a chemical structure such as used here has an arrow coming from it, the arrow indicates the attachment point. For example, the structure

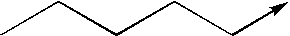

is a pentyl radical. When a value in parentheses appears near the arrow, the value indicates where the attachment point can be found in the compound. For example, in the general

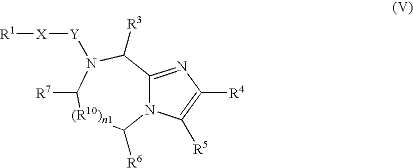

as defined previously, when R¹⁰ and R⁷ are taken together to form the radical

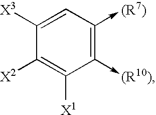

the following structure results:

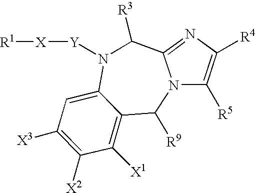

Among the compounds of general formula (V), in particular 1-(2-(1-((4-cyano)phenylmethyl)imidazol-4-yl)-1-oxoethyl-2,5-dihydro-4-(2 methoxyphenyl)imidazo[1,2c][1,4]benzodiazepine, 4-(2-bromophenyl)-1-(2-(1-((4-cyano-3-methoxy)phenylmethyl)imidazo-5-yl)-1-oxoethyl)-1,2-dihydro-8fluoroimidazo[1,2a][1,4]-benzodiazepine or one of its pharmaceutically acceptable salts (and quite particularly 4-(2-bromophenyl)-1-(2-(1-((4-cyano-3methoxy)phenylmethyl)imidazo-5-yl)-1-oxoethyl)-1,2-dihydro-8-fluoroimidazo[1,2a][1,4]-benzodiazepine or one of its pharmaceutically acceptable salts).

As regards the CDK inhibitors, these are preferably chosen from the compounds of general formula (VII)

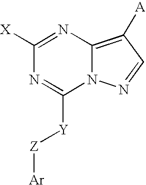

in racemic, enantiomeric form or all combinations of these forms, in which

A represents a hydrogen atom, a halogen atom, a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or also a -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, the complimentary members being chosen independently from the group comprising —CH₂—, —NR³—, —S— and —O—, R³ independently representing each time that it occurs a hydrogen atom or an alkyl radical;

X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by one or more radicals chosen from the alkyl, hydroxy and amino radicals, an aralkyl radical the radical aryl of which is optionally substituted by one or more radicals chosen from a halogen atom, the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by one or more alkyl radicals and R⁵ represents a hydrogen atom, or also R⁴ and R⁵ taken together with the nitrogen atom which carries them form a heterocycle with 5 to 7 members, the complimentary members being chosen independently from the group comprising —CH₂—, —NR⁶—, —S— and —O—, R⁶ independently representing each time that it occurs a hydrogen atom or an alkyl or hydroxyalkyl radical;

Y represents NH or an oxygen atom;

Z represents a bond or an alkyl or alkylthioalkyl radical; and

Ar represents a carbocyclic aryl radical optionally substituted 1 to 3 times by radicals chosen independently from a halogen atom, the cyano radical, the nitro radical, an alkyl or alkoxy radical and an NR⁷R⁸ radical in which R⁷ and R⁸ independently represent a hydrogen atom or an alkyl radical or R⁷ and R⁸ taken together with the nitrogen atom which carries them form a heterocycle with 5 to 7 members, the complimentary members being chosen independently from the group comprising —CH₂—, —NR⁹—, —S— and —O—, R⁹ representing independently each time that it occurs a hydrogen atom or an alkyl radical, or also Ar represents a heterocyclic aryl radical with 5 or 6 members the heteroatoms of which are chosen from nitrogen, oxygen or sulphur atoms, said heteroatoms optionally being oxidized (Ar can represent for example the oxidopyridyl radical) and said heterocyclic aryl radical being able to be optionally substituted by one or more radicals chosen independently from the alkyl, aminoalkyl, alkylaminoalkyl and dialkylaminoalkyl radicals; or the pharmaceutically acceptable salts of these compounds.

According to the invention, the compounds of general formula (VII) (and also their pharmaceutically acceptable salts) are preferably such that they have at least one of the following characteristics:
A represents a halogen atom, a formyl, guanidinoaminomethylenyl, (1,3-dihydro-2oxoindol)-3-ylidenemethyl or alkylcarbonyl radical, or also a -L-NR¹R² radical in which L represents a methylene radical and R¹ and R² are chosen independently from a hydrogen atom and an alkyl radical or R¹ and R² taken together with the nitrogen atom which carries them form a heterocycle with 5 to 7 members, the complimentary members being chosen independently from the group comprising —CH₂—, —NR³— and —O—, R³ independently representing each time that it occurs a hydrogen atom or an alkyl radical;
X represents an alkylthio radical (and preferably methylthio) or alkylthioxo (and preferably methylthioxo), or also an NR⁴R⁵ radical in which R⁴ represents an alkyl radical, a hydroxyalkyl radical, a cycloalkyl radical (and preferably cyclohexyl) optionally substituted by one or more amino radicals, or also R⁴ represents a heteroaryl or heteroarylalkyl radical, the heteroaryl radical of the heteroaryl or heteroarylalkyl radicals being optionally substituted by one or more alkyl radicals and R⁵ represents a hydrogen atom, or also R⁴ and R⁵ taken together with the nitrogen atom which carries them form a heterocycle with 5 to 7 members, the complementary members being chosen independently from the group comprising —CH₂— and —NR⁶—, R⁶ independently representing each time that it occurs a hydrogen atom or an alkyl or hydroxyalkyl radical;
Y represents NH;
Z represents a bond or a —CH₂— radical;
Ar represents a carbocyclic aryl radical (said carbocyclic aryl radical preferably being a phenyl radical) optionally substituted 1 to 3 times by radicals chosen independently from a halogen atom and an NR⁷R⁸ radical in which R⁷ and R⁸ independently represent a hydrogen atom or an alkyl radical or R⁷ and R⁸ taken together with the nitrogen atom which carries them form a heterocycle with 5 to 7 members, the complimentary members being chosen independently from the group comprising —CH₂— and —NR⁹—, R⁹ independently representing each time that it occurs an alkyl radical,
or also Ar represents a heterocyclic aryl radical having 5 or 6 members the heteroatom or heteroatoms of which are chosen from nitrogen and oxygen atoms (said heterocyclic aryl radical preferably being a pyridyl radical), said heteroatoms optionally being oxidized and said heterocyclic aryl radical being able to be optionally substituted by one or more radicals chosen independently from the alkyl, aminoalkyl, alkylaminoalkyl and dialkylaminoalkyl radicals.

Among the compounds of general formula (VII), in particular the compounds chosen from the group constituted by the following compounds will be preferred:
bromo-4-[(3-pyridyl)methylamino]-2-methylthio-pyrazolo[1,5-a]-1,3,5-triazine;
8-bromo-2-(1R-isopropyl-2-hydroxyethylamino)-4-(3-fluorophenylmethylamino)-pyrazolo[1,5-a]-1,3,5-triazine;
8-bromo-2-(1R-isopropyl-2-hydroxyethylamino)-4-(3-pyridylmethylamino)pyrazolo[1,5-a]-1,3,5-triazine;

and their pharmaceutically acceptable salts.

The CDK inhibitors can alternatively be chosen from roscovitine and its analogues, or also from olomoucine, purvalanol, the compound known by the name CVT-313, flavopiridol, γ-butyrolactone, indirubins, paullones and staurosporine (cf. Gray et al., Curr. Med. Chem. (1999), 6(9), 859-75 and cited references).

A further subject of the invention is a particularly useful compound of general formula (IV), i.e. (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine, or its pharmaceutically acceptable salts.

This compound and its salts have shown particularly stable in the form of a powder.

The major advantage of this compound is however its potent anti-cancer activity (whether alone or in combination with other anti-cancer agents) combined with excellent in vivo toxicity data. Besides, this compound is also a potent anti-pain agent, which is also a desirable feature for an anti-cancer agent.

Moreover, a particularly preferred salt of this compound is (1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine tetrahydrochloride.

The invention also offers a very convenient and economical preparation process for said tetrahydrochloride salt, said process comprising the following steps:
1) reacting approximately 2 equivalents of (8S)-8-(cyclohexylmethyl)-2-phenyl-5,6,7,8-tetrahydroimidazo[1,2-a]pyrazine with approximately one equivalent of Boc-Cys-Cys-Boc in a polar aprotic solvent; and
2) reacting in a lower alcohol the disulphide derivative obtained after stage 1) with an excess of hydrochloric acid in solution in a lower alcohol.

By polar aprotic solvent should be understood in the abovementioned process dimethylformamide or tetrahydrofuran, and preferably dimethylformamide.

By excess of hydrochloric acid should be understood in the abovementioned process at

least 4 equivalents of hydrochloric acid (e.g. from 4 to 5 equivalents of hydrochloric acid).

By lower alcohol should be understood an alcohol comprising from 1 to 4 carbon atoms, notably methanol, ethanol or isopropanol. A preferred lower alcohol for stage 2) of the abovementioned process is isopropanol.

Preferably, the addition of the hydrochloric acid solution at stage 2) will be carried out at a temperature not exceeding 25° C. (and more preferably at a temperature not exceeding 5° C.).

Optionally, the reaction medium will be cooled down (e.g. at a temperature of about 0° C. to isolate the expected terahydrochloride salt by crystallization.

Amongst the cancers intended to be treated by a product according to the invention, in particular cancer of the breast, lymphomes, cancers of the neck and the head, cancer of the lung, cancer of the colon, cancer of the prostate and cancer of the pancreas can be mentioned.

A subject of the invention is also a method for treating cancer, said method comprising the administration of a therapeutically effective dose of a product according to the invention to the patient in need of this treatment.

The pharmaceutical compositions containing a product of the invention can be presented in the form of solids, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups.

Appropriate liquid supports can be, for example, water, organic solvents such as glycerol or glycols, as well as their mixtures, in varying proportions, in water.

The administration of a medicament according to the invention can be carried out by topical, oral, parenteral route, by intramuscular injection, etc.

The administration dose envisaged for a medicament according to the invention is comprised between 0.1 mg and 10 g according to the type of active compound used.

In accordance with the invention, the compounds included in products according to the invention can be prepared for example by the processes described below.

### Preparation of Certain Compounds Included in Products According to the Invention

### Preparation of the Compounds of General Formula (I)

The preparation processes below are given by way of illustration and a person skilled in the art will be able to subject them to the variations that he judges useful, just as easily as regards the reagents as the conditions and techniques of the reactions.

### General Method

Generally, the compounds of general formula (I) can be prepared according to the procedure summarized in Diagram 1 below.

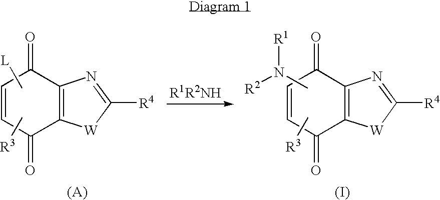

According to this method, the compounds of general formula (I), in which R¹, R², R³, R⁴ and W are as described above, are obtained by treating the compounds of general formula (A), in which L represents a methoxy radical, a halogen atom or a hydrogen atom and R³, R⁴ and W have the same meaning as in general formula (I), with amines of general formula NR¹R²H in a protic solvent such as methanol or ethanol, at a temperature comprised between 0° C. and 50° C. and optionally in the presence of a base such as, for example, diisopropylethylamine (Yasuyuki Kita et al., J. Org. Chem. (1996), 61, 223-227).

In the particular case where the compounds of general formula (A) are such that L and R³ each represent a halogen atom, the compounds of general formula (I) can be obtained in the form of a mixture of the 2 position isomers, but it is then possible to separate them by chromatography on a silica column in an appropriate eluent.

Alternatively, the compounds of general formula (I) in which R³ represents a halogen atom (Hal) can be obtained, diagram 1a, from the compounds of general formula (I) in which R³ represents a hydrogen atom, for example by the action of N-chlorosuccinimide or N-bromosuccinimide in an aprotic solvent such as dichloromethane or tetrahydrofuran (Paquette and Farley, J. Org. Chem. (1967), 32, 2725-2731), by the action of an aqueous solution of sodium hypochlorite (bleach) in a solvent such as acetic acid (Jagadeesh et al., Synth Commun. (1998), 28, 3827-3833), by the action of Cu(II) (in a CuCl₂/HgCl₂ mixture) in the presence of a catalytic quantity of iodine in a solvent such as warm acetic acid (Thapliyal, Synth. Commun. (1998), 28, 1123-1126), by the action of an agent such as benzyltrimethylammonium dichloroiodate in the presence of NaHCO₃ in a solvent such as a dichloromethane/methanol mixture (Kordik and Reitz, J. Org. Chem. (1996), 61, 5644-5645), or also by using chlorine, bromine or iodine in a solvent such as dichloromethane (J. Renault, S. Giorgi-Renault et al., J. Med. Chem. (1983), 26, 1715-1719).

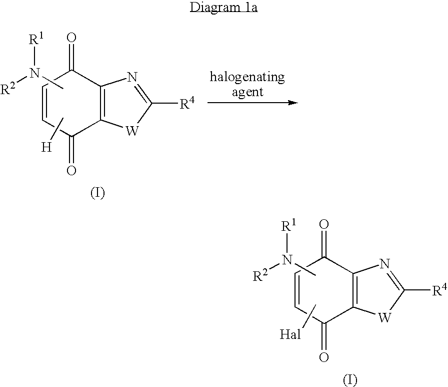

Alternatively also, the compounds of general formula (I) in which R³ represents an alkoxy or alkylthio radical can be obtained, Diagram 1b, from the compounds of general formula (I) in which R³ represents a halogen atom, for example, by the action of an alcohol of general formula R^{3′}—OH or a thiol of general formula R^{3′}—SH (R^{3′} being such that R³═R^{3′}O or R^{3′}S) in a solvent such as anhydrous ethanol in the presence of a base such as, for example, diisopropylethylamine.

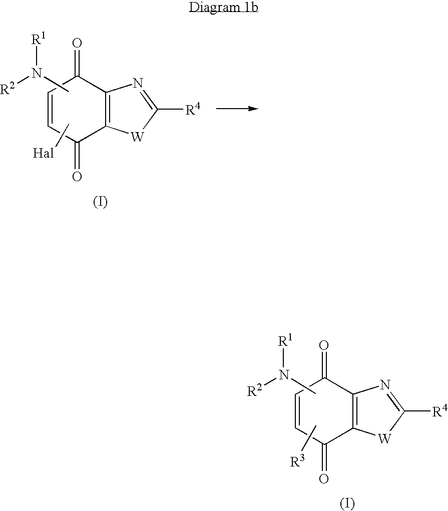

### Preparation of the Intermediates of General Formula (A)

The compounds of general formula (A) in which L, R³, R⁴ and W are as defined above can be obtained, Diagram 2, from the compounds of general formula (B) in which L, R³, R⁴ and W are as defined above and:

one of Q and Q′ represents an amino or hydroxyl radical and the other represents a hydrogen atom; or

Q and Q′ each represent an amino radical; or

Q and Q′ each represent a hydroxyl radical; or finally

Q and Q′ each represent a methoxy radical.

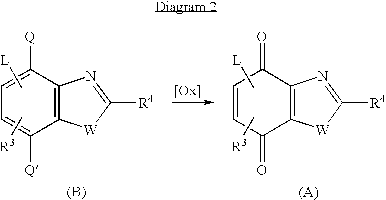

In the case where the compounds of general formula (B) are such that Q and Q′ represent methoxy radicals, the compounds of general formula (A) are obtained by treatment with cerium (IV) and ammonium nitrate (Beneteau et al., Eur. J. Med. Chem. (1999), 34(12), 1053-1060). In the other cases, the compounds of general formula (A) are obtained by oxidation of the compounds of general formula (B), for example by the use of FeCl₃ in an acid medium (Antonini et al., Heterocycles (1982), 19(12), 2313-2317) or Fremy's salt (potassium nitrosodisulphonate). (Ryu et al., Bioorg. Med. Chem. Lett. (2000), 10, 461-464), or by the use of a reagent comprising a hypervalent iodine such as [bis(acetoxy)iodo]benzene or [bis(trifluoroacetoxy)iodo]benzene in aqueous acetonitrile at a temperature preferably comprised between −20° C. and ambient temperature (i.e. approximately 25° C.), and preferably at approximately −5° C. (Kinugawa et al., Synthesis, (1996), 5, 633-636).

In the particular case where L and R³ represent halogens atoms, the compounds of general formula (A) can be obtained, Diagram 3, by halogen oxidation of the compounds of general formula (B) in which L and R³ represent hydrogen atoms and Q and/or Q′ is (are) chosen from an amino radical and a hydroxy radical by the action, for example, of potassium or sodium perchlorate in acid medium (Ryu et al., Bioorg. Med. Chem. Lett. (1999), 9, 1075-1080).

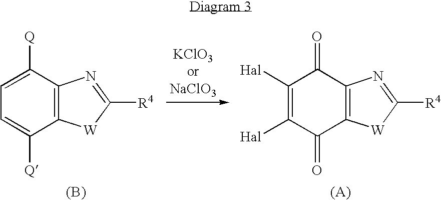

### Preparation of the Intermediates of General Formula (B)

Certain compounds of general formula (B) in which L, R³, R⁴, Q, Q′ and W are as defined above are known industrial products available from the usual suppliers.

If they are not commercially available and in the particular case where Q or Q′ represents an amino radical, the compounds of general formula (B) can in particular be obtained from the nitro derivatives of formula (B.ii) in which Q or Q′ represents a nitro radical by reduction methods which are well known to a person skilled in the art such as, for example, hydrogenation in the presence of a palladium catalyst or treatment with tin chloride in hydrochloric acid. If they are not commercially available, the compounds of formula (B.ii) can themselves be obtained from the compounds of general formula (B.i) in which the positions corresponding to the Q and Q′ radicals are substituted by hydrogen atoms by nitration methods which are well known to the person skilled in the art such as, for example, treatment with a mixture of nitric acid and sulphuric acid (cf. Diagram 4 where only the case in which the compounds of general formula (B) are such that Q=NH₂ and Q′=H is represented).

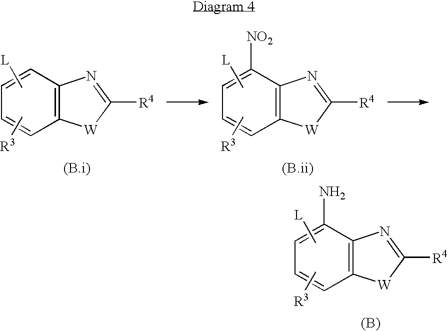

Alternatively, the compounds of general formula (B) which are not commercially available in which Q represents an amino radical, Q′ a hydrogen atom and W an oxygen atom, can be obtained by treatment of the tetrahydrobenzoxazoles of general formula (B.vi) with hydroxylamine hydrochloride in order to produce the oximes of general formula (B.v), themselves treated with warm polyphosphoric acid (cf. Young Kook Koh et al., J. Heterocyclic Chem. (2001), 38, 89-92) to provide the compounds of general formula (B). The compounds of general formula (B.vi) can themselves be obtained from the cyclic 1,3-diketones of general formula (B.viii) firstly by conversion to diazodiketones of general formula (B.vii) by diazotransfer reaction, for example, by the action of tosyl azide or 4-acetamidobenzenesulphonylazide in the presence of triethylamine in a solvent such as anhydrous dichloromethane or chloroform (V. V. Popic et al., Synthesis (1991), 3, 195-198) followed by cycloaddition of these diazodiketones of general formula (B.vii) with the nitriles of general formula R⁴—CN in the presence of a rhodium (II)-type catalyst (Y. R. Lee, Heterocycles (1998), 48, 875-883) (cf. Diagram 4a).

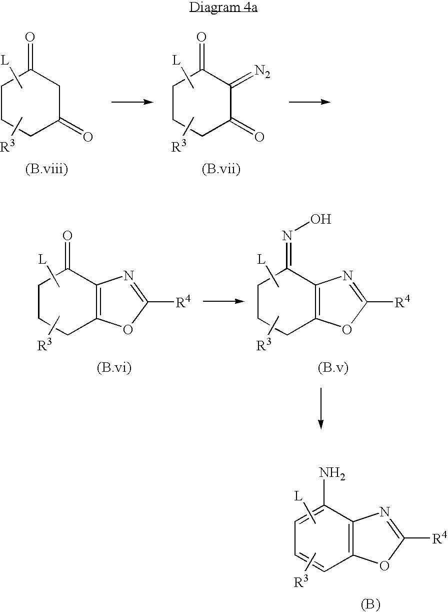

If they are not commercially available and in the particular case where Q represents hydroxyl, Q′ a hydrogen atom and W an oxygen atom, the compounds of general formula (B) can be obtained by aromatization of the oxazolocyclohexanones of general formula (B.vi). Such an aromatization can be carried out in two stages as shown in Diagram 4b, firstly a halogenation in position α of the carbonyl (which leads to the intermediates of general formula (B.ix) in which Hal is a halogen atom), then β-elimination of the halogen by treatment with a base. The halogenation can be carried out, for example, using bromine in acetic acid at ambient temperature, pyridinium tribromide in acetic acid at 50° C., copper bromide (II) in ethyl acetate or acetonitrile under reflux, or also phenylselenyl chloride in ethyl acetate at ambient temperature.

The elimination of the resulting halide can be carried out with diazabicyclo[5.4.0]undec-7-ene (DBU) in tetrahydrofuran at ambient temperature or with lithium carbonate in dimethylformamide. Examples of these reactions are provided by M. Tany et al., Chem. Pharm. Bull. (1996), 44, 55-61; M. A. Ciufolini et al., J. Am. Chem. Soc. (1995), 117, 12460-12469; and M. E. Jung and L. S. Starkey, Tetrahedron (1997), 53, 8815-8824.

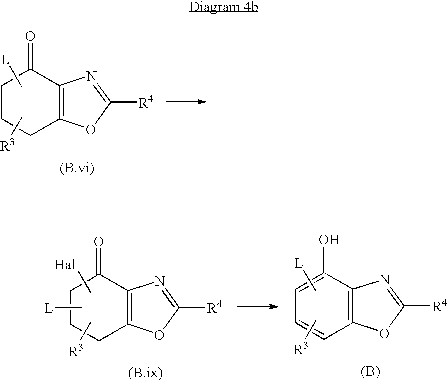

If they are not commercially available and in the particular case where R⁴ represents a —CH₂—NR²¹R²² radical, the compounds of general formula (B) can be obtained, Diagram 5, from the compounds of general formula (B.iii) in which R⁴ represents the methyl radical, which is firstly subjected to a radical bromination using N-bromosuccinimide in the presence of an initiator such as 2,2′-azobis(2-methylpropionitrile) or dibenzoylperoxide in an aprotic solvent such as carbon tetrachloride (CCl₄) at a temperature preferably comprised between ambient temperature (i.e. approximately 25° C.) and 80° C. and with irradiation by a UV lamp (Mylari et al., J. Med. Chem. (1991), 34, 108-122), followed by a substitution of the intermediate of general formula (B.iv) with amines of formula HNR²¹R²² with R²¹ and R²² being as defined above.

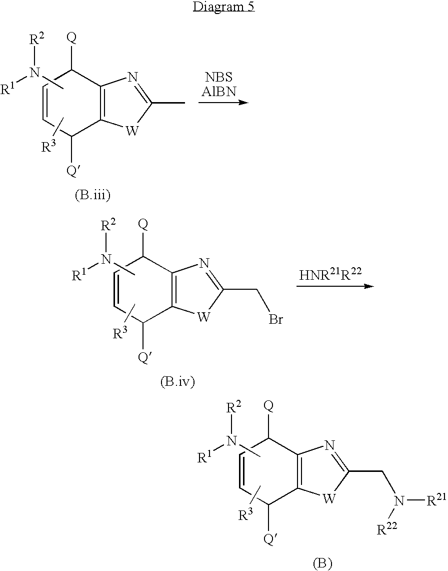

Alternatively, the compounds of general formula (B) which are not commercially available in which R⁴ represents a —CH₂—NR²¹R²² radical can be obtained according to the method represented in Diagram 4 above, from compounds of general formula (B.1) in which R⁴ represents a —CH₂—NR²¹R²² radical, these being themselves obtained from the compounds of general formula (B.1) in which R⁴ represents a CH₂—Br radical by substitution with amines of formula HNR²¹R²² with R²¹ and R²² as defined above. The compounds of general formula (B.1) in which R⁴ represents a CH₂—Br radical can be obtained, as described above, from the compounds of general formula (B.1) in which R⁴ represents the methyl radical, which is subjected to a radical bromination reaction.

If they are not commercially available and in the particular case where R⁴ represents a —CH₂—CO—NR¹⁹R²⁰ radical, the compounds of general formula (B) can be obtained from the compounds of general formula (B) in which R⁴ represents the —CH₂—COOH radical, by standard methods of peptide synthesis (M. Bodansky, The Practice of Peptide Synthesis, 145 (Springer-Verlag, 1984)), for example in tetrahydrofuran, dichloromethane or dimethylformamide in the presence of a coupling reagent such as cyclohexylcarbodiimide (DCC), 1,1′-carbonyldiimidazole (CDI) (J. Med. Chem. (1992), 35(23), 4464-4472) or benzotriazol-1-yl-oxy-tris-pyrrolidino-phosphonium hexafluorophosphate (PyBOP) (Coste et al., Tetrahedron Lett. (1990), 31, 205).

The compounds of general formula (B) in which R⁴ represents —CH₂—COOH can be obtained from the compounds of general formula (B) in which R⁴ represents the —CH₂COOR¹⁸ radical in which R¹⁸ represents an alkyl radical by hydrolysis of the ester function under conditions known to a person skilled in the art.

The compounds of general formula (B) in which W represents S, Q and Q′ each represent a methoxy radical and L represents a halogen atom or a hydrogen atom can be obtained, Diagram 6, by treating N-(2,5-dimethoxyphenyl)thioamides of general formula (B.x) with an aqueous solution of potassium ferricyanide in a sodic medium at ambient temperature (Lyon et al., J. Chem. Soc., Perkin Trans. 1 (1999), 437-442). The compounds of general formula (B.x) can themselves be obtained starting from corresponding acylated 2,5-dimethoxyanilines of general formula (B.xii), for example by the action of an acid chloride of general formula R⁴COCl or a carboxylic acid of general formula R⁴COOH activated according to methods known to the person skilled in the art, in order to produce N-(2,5-dimethoxyphenyl)amides of general formula (B.xi) themselves converted to the thioamides of general formula (B.x) by the action of Lawesson's reagent in toluene under reflux.

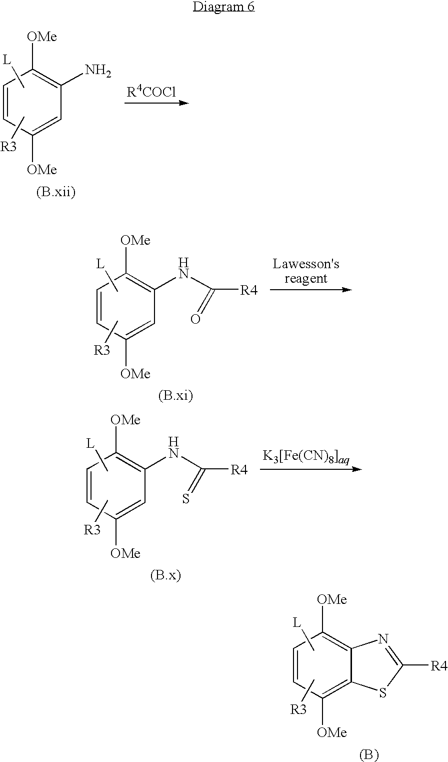

In the other cases, the compounds of general formula (B) can be obtained, Diagram 6a, from the compounds of general formula (C) in which L, R³ and W are as defined above and Q or Q′ represents the NO₂ radical by condensation with the orthoester of general formula R⁴C(OR)₃ in which R is an alkyl radical, for example in the presence of a catalytic quantity of an acid such as, for example, paratoluenesulphonic acid, at a temperature comprised between ambient temperature and 200° C. and preferably at approximately 110° C. (Jenkins et al., J. Org. Chem. (1961), 26, 274) or also in a protic solvent such as ethanol at a temperature comprised between ambient temperature (i.e. approximately 25° C.) and 80° C. and preferably at approximately 60° C. (Scott et al., Synth. Commun. (1989), 19, 2921). A certain number of orthoesters are known industrial products available from the usual suppliers. The preparation of orthoesters for treating various nitrile compounds with hydrochloric gas in an alcohol is known to a person skilled in the art.

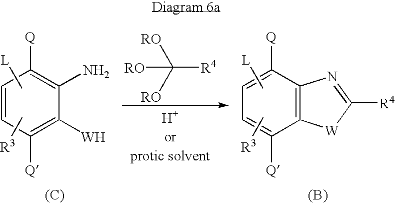

The compounds of general formula (B) in which L, R³, R⁴ and W are as defined above and Q or Q′ represents the NO₂ radical can also be obtained from the compounds of general formula (C) in which L, R³, R⁴ and W are as defined above and one of Q and Q′ represents the NO₂ radical while the other represents a hydrogen atom by condensation of the latter with an acid chloride of formula R⁴—COCl under an inert atmosphere and in a polar and slightly basic solvent such as N-methyl-2-pyrrolidinone (Brembilla et al., Synth. Commun (1990), 20, 3379-3384) or by condensation of the latter with a carboxylic acid of general formula R⁴—COOH in the presence of polyphosphoric acid at high temperature (Ying-Hung So et al., Synth. Commun. (1998), 28, 4123-4135) or in the presence of boric acid in a solvent such as xylene under reflux (M. Terashima, Synthesis (1982), 6, 484-485).

The compounds of general formula (B) in which L, R³, R⁴ and W are as defined above and Q or Q′ represents the NO₂ radical can also be obtained from the compounds of general formula (C) in which L, R³, R⁴ and W are as defined above and one of Q and Q′ represents the NO₂ radical while the other represents a hydrogen atom by condensation with an aldehyde of general formula R⁴—CHO then treating the Schiff base obtained with an oxidizing agent such as [bis(acetoxy)iodo]benzene, ferric chloride or dimethylsulphoxide (Racane et al., Monatsh. Chem. (1995), 126(12), 1375-1381) or by dehydrating with glacial acetic acid at a temperature comprised between ambient temperature (i.e. approximately 25° C.) and 100° C. (Katritzky and Fan, J. Heterocyclic Chem. (1988), 25, 901-906).

The compounds of general formula (B) in which L, R³, R⁴ and W are as defined above and one of Q and Q′ represents the NO₂ radical while the other represents a hydrogen atom can also be obtained from the compounds of general formula (C) by condensation with a nitrile of general formula R⁴—CN in a mixture of solvents of methanol/glacial acetic acid type at a temperature comprised between ambient temperature (i.e. approximately 25° C.) and 100° C. (Nawwar and Shafik, Collect. Czech Chem. Commun. (1995), 60(12), 2200-2208).

### Preparation of the Intermediates of General Formula (C)

Certain compounds of general formula (C) in which L, R³, Q, Q′ and W are as defined above are known industrial products available from the usual suppliers.

Certain compounds of general formula (C) in which one of Q and Q′ represents the NO₂ radical while the other represents a hydrogen atom can be obtained from the compounds of general formula (D)

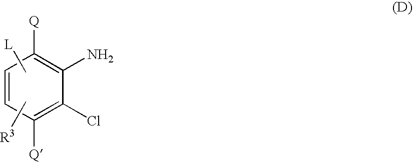

in which L, R³, Q and Q′ are as defined above by reaction, in the case where W represents S, with hydrated sodium sulphide at a temperature comprised between ambient temperature (i.e. approximately 25° C.) and 100° C. (Katritzky and Fan, J. Heterocyclic Chem. (1988), 25, 901-906).

Finally, in the particular case where W represents O, the compounds of general formula (C) are known industrial products available from the usual suppliers or can be synthesized from such products according to current methods known to a person skilled in the art.

### Separation of Mixtures of Regioisomers

In certain cases, it can happen that the compounds of general formula (I) prepared according to the above-mentioned methods are obtained in the form of mixtures of regioisomers.

In such situations, the mixture can be separated with standard liquid chromatography techniques on a column or on preparative thin layer (using a support such as silica or also a gel such as a gel of cross-linked polydextrans forming a three-dimensional network such as a Sephadex® LH-20 type gel). The person skilled in the art will choose the eluent the best suited to the separation of the mixture; such an eluent can be for example a ternary isopropanol/ethyl acetate/water mixture 1/1/1.

### Preparation of the Compounds of General Formula (II)

The compounds of general formula (II) have been described in PCT Patent Application WO 02/09686.

### Preparation of the Compounds of General Formula (III)

The compounds of general formula (III) have in particular been described in PCT Patent Application WO 97/00876.

### Preparation of the Compounds of General Formula (IV)

The compounds of general formula (IV) have been described in PCT Patent Application WO 97/30053.

The most preferred compound, bis-1,1′-{7-(2-amino-1-oxo-3-thiopropyl)-8-(cyclohexylmethyl)-2-phenyl-5,6,7,8-tetrahydroimidazo[1,2a]pyrazine}disulphide, can alternatively be prepared according to the 2-step process represented in Diagram 7 hereafter.

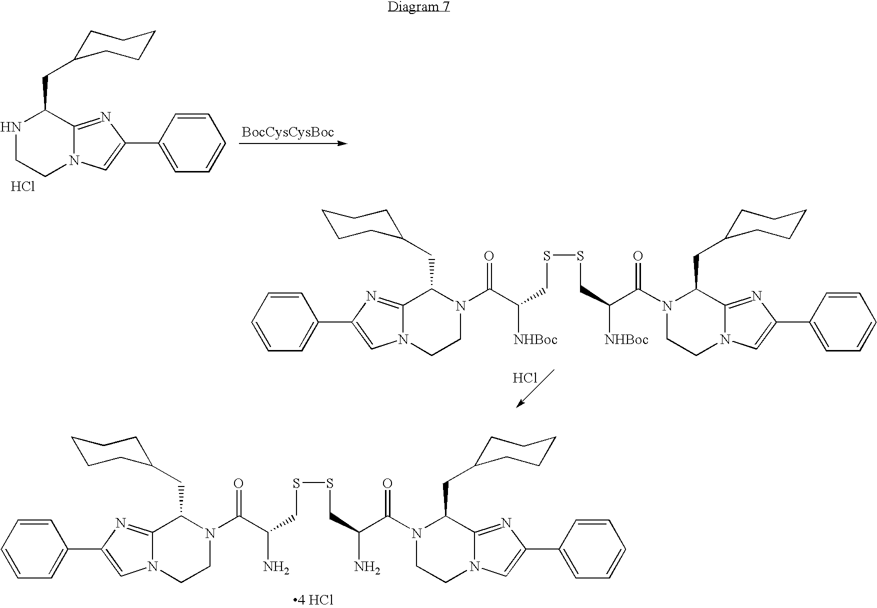

According to this method, (8S)-8-(cyclohexylmethyl)-2-phenyl-5,6,7,8-tetrahydroimidazo[1,2-a]pyrazine (possibly in the form of its hydrochloride salt; product obtained according to the protocol described in PCT application WO 97/30053) can be first condensed with one equivalent of Boc-Cys-Cys-Boc in the presence of a peptidic coupling agent (e.g. HTBU) and of a base (e.g. diisopropylethylamine). The intermediate compound can then be deprotected and converted into the desired tetrahydrochloride salt in one single step by addition of a HCl solution in a lower alcohol (e.g. isopropanol), this reaction being preferably carried out in the same lower alcohol.

### Preparation of the Compounds of General Formula (V)

The compounds of general formula (V) have been described in PCT Patent Application WO 00/39130.

### Preparation of the Compounds of General Formula (VI)

The compounds of general formula (VI) have been described in PCT Patent Application WO 97/21701.

### Preparation of the Compounds of General Formula (VII)

The compounds of general formula (VII) have been described in PCT Patent Application WO 02/50073.

As regards the temperatures referred to in the present text, the term <<approximately XX° C.>> indicates that the temperature in question corresponds to a range of more or less 10° C. either side of the temperature XX° C., and preferably to a range of more or less 5° C. either side of the temperature XX° C. As regards the other numerical values referred to in the present text, the term <<approximately YY>> indicates that the value in question corresponds to a range of more or less 10% either side of the value YY, and preferably to a range of more or less 5% either side of the value YY.

Unless they are defined otherwise, all the technical and scientific terms used here have the same meaning as that usually understood by an ordinary specialist in the field to which this invention belongs. Similarly, all the publications, patent applications, all the patents and all other references mentioned here are incorporated by way of reference.

The following examples are presented in order to illustrate the above procedures and should in no event be considered as a limit to the scope of the invention.

### EXAMPLES OF COMPOUNDS OF GENERAL FORMULA (I)

### Method Used for Measuring the Retention Time (r.t.) and the Molecular Peak (MH+)

The compounds are characterised by their retention time (r.t.), expressed in minutes, determined by liquid chromatography (LC), and their molecular peak (MH+) determined by mass spectrometry (MS), a single quadripole mass spectrometer (Micromass, Platform model) equipped with an electrospray source is used with a resolution of 0.8 Da at 50% valley. For Examples 1 to 138 below, the elution conditions corresponding to the results indicated are the following: transition of an acetonitrile-water-trifluoroacetic acid mixture 50-950-0.2 (A) to an acetonitrile-water mixture 950-50 (B) via a linear gradient over a period of 8.5 minutes, then elution with the pure mixture B for 10.5 minutes.

### Example 1

### 2-methyl-5-{[2-(4-morpholinyl)ethyl]amino}-1,3-benzothiazole-4,7-dione

51.2 μl (0.39 mmol; 3 equivalents) of 4-(2-aminoethyl)morpholine is added to 27 mg (0.129 mmol) of 5-methoxy-2-methyl-4,7-dioxobenzothiazole in solution in 2 ml of anhydrous ethanol. The reaction mixture is stirred under reflux for 18 hours then the solvent is evaporated off under reduced pressure. The residue is purified on a silica column (eluent: 5% methanol in dichloromethane). The expected compound is obtained in the form of a red powder.

NMR ¹H (DMSO d6, 400 MHz, δ): 7.45 (t, 1H, NH); 5.49 (s, 1H, CH); 3.58-3.55 (m, 4H, 2 CH₂); 3.26 (t, 2H, CH₂); 2.75 (s, 3H, CH₃); 2.54 (t, 2H, CH₂); 2.42-2.40 (m, 4H, 2 CH₂).

MS-LC: MH+=308.25; r.t.=6.89 min.

### Example 2

### 5-{[2-(dimethylamino)ethyl]amino}-2-methyl-1,3-benzothiazole-4,7-dione hydrochloride

### 2.1) 5-{[2-(dimethylamino)ethyl]amino}-2-methyl-1,3-benzothiazole-4,7-dione

This compound is obtained in a similar manner to that used for the compound of Example 1.

NMR ¹H (DMSO d6, 400 MHz, δ): 7.34 (t, 1H, NH); 5.48 (s, 1H, CH); 3.24-3.20 (m, H, CH₂); 2.77 (s, 3H, CH₃); 2.47 (m, 2H, CH₂); 2.18 (s, 6H, 2 CH₃). MS-LC: MH+=266.27; r.t.=6.83 min.

### 2.2)₅-{[2-(dimethylamino)ethyl]amino}-2-methyl-1,3-benzothiazole-4,7-dione hydrochloride

0.166 g of intermediate 2.1 is dissolved in 1.88 ml (1.88 mmol; 3 eq.) of a molar solution of hydrochloric acid in ether and the reaction mixture is stirred for 3 hours at ambient temperature. The resulting precipitate is collected by filtration, followed by washing with ethyl ether and drying under reduced pressure in order to produce a dark red solid. Melting point: 138-140° C.

NMR ¹H (DMSO d6, 400 MHz, δ): 10.00 (s, 1H, NH⁺); 7.78 (t, 1H, NH); 5.68 (s, 1H, CH); 3.59-3.55 (m, 2H, CH₂); 3.32-3.27 (m, 2H, CH₂); 2.85-2.80 (s, 6H, 2 CH₃); 2.76 (s, 3H, CH₃).

MS-LC: MH+=266.12; r.t.=6.92 min.

The compounds of Examples 3 to 14 are obtained in a similar manner to that used for Example 1.

### Example 3

### 5-{[6-(dimethylamino)hexyl]amino}-2-methyl-1,3-benzothiazole-4,7-dione

MS-LC: MH+=322.33; r.t.=7.36 min.

### Example 4

### 5-{[3-(dimethylamino)-2,2-dimethylpropyl]amino}-2-methyl-1,3-benzothiazole-4,7-dione

NMR ¹H (DMSO d6, 400 MHz, δ): 8.62 (t, 1H, NH); 5.45 (s, 1H, CH); 3.07-3.06 (m, 2H, CH₂); 2.74 (s, 3H, CH₃); 2.29-2.30 (m, 2H, CH₂); 2.27 (s, 6H, 2CH₃); 0.93 (s, 6H, 2 CH₃).

LC-MS: MH+=308.32; r.t.=7.16 min.

### Example 5

### 2-methyl-5-{[3-(4-methyl-1-piperazinyl)propyl]amino}-1,3-benzothiazole-4,7-dione

NMR ¹H (DMSO d6, 400 MHz, δ): 8.14 (t, 1H, NH); 5.46 (s, 1H, CH); 3.25-3.26 (m, 2H, CH₂); 3.21-3.19 (m, 2H, CH₂); 2.74 (s, 3H, CH₃); 2.49-2.48 (m, 2H, CH₂); 2.37-2.32 (m, 6H, 3CH₂); 2.16 (s, 3H, CH₃); 1.72 (t, 2H, CH₂).

MS-LC: MH+=335.34; r.t.=6.87 min.

### Example 6

### 5-[(1-ethylhexyl)amino)-2-methyl-1,3-benzothiazole-4,7-dione

MS-LC: MH+=307.32; r.t.=11.45 min.

### Example 7

### 5-[(1-adamantylmethyl)amino]-2-methyl-1,3-benzothiazole-4,7-dione

MS-LC: MH+=343.31; r.t.=11.73 min.

### Example 8

### 2-methyl-5-[(2-thienylmethyl)amino]-1,3-benzothiazole-4,7-dione

MS-LC: MH+=291.16; r.t.=9.24 min.

### Example 9

### 5-[(3-chlorobenzyl)amino]-2-methyl-1,3-benzothiazole-4,7-dione

MS-LC: MH+=319.24; r.t.=9.95 min.

### Example 10

### 2-methyl-5-[(4-pyridinylmethyl)amino]-1,3-benzothiazole-4,7-dione

MS-LC: MH+=286.13; r.t.=6.97 min.

### Example 11

### 2-methyl-5-(propylamino)-1,3-benzothiazole-4,7-dione

MS-LC: MH+=237.16; r.t.=8.74 min.

### Example 12

### 5-{[3-(1H-imidazol-1-yl)propyl]amino}-2-methyl-1,3-benzothiazole-4,7-dione

MS-LC: MH+=303.17; r.t.=7.07 min.

### Example 13

### 4-{2-[(2-methyl-4,7-dioxo-4,7-dihydro-1,3-benzothiazol-5-yl)amino]ethyl}benzenesulphonamide

MS-LC: MH+=378.10; r.t.=8.31 min.

### Example 14

### 5-(4-benzyl-1-piperazinyl)-2-methyl-1,3-benzothiazole-4,7-dione

MS-LC: MH+=354.19; r.t.=7.53 min.

### Example 15

### 5-anilino-2-ethyl-1,3-benzoxazole-4,7-dione or 6-anilino-2-ethyl-1,3-benzoxazole-4,7-dione

### 15.1) 2-ethyl-4-nitro-1,3-benzoxazole

A mixture of 2-amino-3-nitrophenol (1 eq.), triethyl orthopropionate (2 eq.) and p-toluene sulphonic acid (in a catalytic quantity) is stirred at 110° C. until disappearance of the aminophenol is verified by thin layer chromatography (2 hours). After cooling down, the reaction mixture is taken up in toluene followed by evaporating under vacuum then treating with isopropanol. The resulting precipitate is collected by filtration, followed by washing with isopropanol and isopentane, then drying under reduced pressure in order to produce a violet-brown solid.

NMR ¹H (DMSO d6, 400 MHz, δ): 8.15 (dd, 2H); 7.58 (t, 1H); 3.06 (q, 2H); 1.38 (t, 3H).

MS-LC: MH+=193.02; r.t.=9.23 min.

### 15.2) 2-ethyl-1,3-benzoxazol-4-amine

2-ethyl-4-nitro-1,3-benzoxazole is hydrogenated under a pressure of 8 bars in the presence of 10% palladium on carbon (0.01 eq.) using methanol as a solvent. The catalyst is separated by filtration and the methanol is eliminated under reduced pressure. The residue is taken up in ethyl ether in order to produce a pale violet solid which is collected by filtration and dried. Melting point: 46° C.

NMR ¹H (DMSO d6, 400 MHz, δ): 6.97 (t, 1H); 6.72 (d, 1H); 6.47, d, 1H); 5.45 (s, 2H); 2.87 (q, 2H); 1.32 (t, 3H).

MS-LC: MH+=162.99; r.t.=8.72 min.

### 15.3) 2-ethyl-1,3-benzoxazole-4,7-dione

A solution of [bis(trifluoroacetoxy)iodo]benzene (2.2 eq.) in a mixture of acetonitrile and water (80/20) is added dropwise to a solution of 2-ethyl-1,3-benzoxazol-4-amine (1 eq.) in the same acetonitrile/water mixture maintained at −5° C. The reaction medium is then diluted with water followed by extracting with dichloromethane. The resulting organic phase is washed with water, followed by drying over sodium sulphate and concentrating in order to produce a brown paste. Purification by medium pressure chromatography on silica gel produces, after taking up in diisopropyl ether, a yellow crystalline solid. Melting point: 99° C.

NMR ¹H(CDCl₃, 400 MHz, δ): 6.75 (dd, 2H); 2.99 (q, 2H); 1.45 (t, 3H).

MS-LC: MH+=177.83; r.t.=8.29 min.

### 15.4) 5-anilino-2-ethyl-1,3-benzoxazole-4,7-dione or 6-anilino-2-ethyl-1,3-benzoxazole-4,7-dione

A mixture of 2-ethyl-1,3-benzoxazole-4,7-dione (1 eq) and aniline (1.1 eq.) in ethanol is kept under stirring for 1 hour. The reaction medium turns to dark violet. After concentration, the residue is purified by medium pressure chromatography on silica in order to produce a violet-coloured powder. Melting point: 200° C.

NMR ¹H (DMSO d6, 400 MHz, δ): 9.38 (s, 1H); 7.44 (t, 2H); 7.36 (d, 2H); 7.22 (t, 1H); 5.69 (s; 1H); 2.94 (q, 2H); 1.29 (t, 3H).

MS-LC: MH+=269.11; r.t.=9.76 min.

### Example 16

### 5-anilino-6-chloro-2-ethyl-1,3-benzoxazole-4,7-dione or 6-anilino-5-chloro-2-ethyl-1,3-benzoxazole-4,7-dione

A solution of 5-anilino-2-ethyl-1,3-benzoxazole-4,7-dione (1 eq.) in acetic acid is treated with N-chlorosuccinimide (1.1 eq.) at ambient temperature. The reaction medium is maintained under stirring for 2 hours before being concentrated, followed by taking up in ethanol and concentrating again. The residue is purified by medium pressure chromatography on silica in order to produce a violet-coloured powder. Melting point: 159° C.

NMR ¹H (DMSO d6, 400 MHz, δ): 9.39 (s, 1H); 7.30 (t, 2H); 7.11 (m, 3H); 2.96 (q, 2H); 1.30 (t, 3H).

MS-LC: MH+=303.01; r.t.=10.28 min.

### Example 17

### 2-ethyl-5-[(4-fluorophenyl)amino]-1,3-benzoxazole-4,7-dione or 2-ethyl-6-[(4-fluorophenyl)amino]-1,3-benzoxazole-4,7-dione

The experimental protocol used is identical to that described for Example 15, 4-fluoroaniline acid replacing aniline in the fourth and last stage. Melting point: 232° C. NMR ¹H (DMSO d6, 400 MHz, δ): 9.38 (s, 1H); 7.37 (t, 2H); 7.26 (t, 2H); 5.57 (s, 1H); 2.93 (q, 2H); 1.30 (t, 3H).

MS-LC: MH+=287.09; r.t.=9.88 min.

The compounds of Examples 18 to 31 are obtained in a similar manner to that described for Example 1.

### Example 18

### 5-[(2-methoxyethyl)amino]-2-methyl-1,3-benzothiazole-4,7-dione

MS-LC: MH+=253.20; r.t.=8.00 min.

### Example 19

### 2-methyl-5-[(2-pyrrolidin-1-ylethyl)amino]-1,3-benzothiazole-4,7-dione

NMR ¹H (DMSO d6, 400 MHz, δ): 7.45 (m, 1H, NH); 5.47 (s, 1H, CH); 3.28-3.23 (m, 2H, CH₂); 2.75 (s, 3H, CH₃); 2.66-2.63 (m, 2H, CH₂); 2.48-2.49 (m, 4H, 2CH₂); 1.68-1.67 (m, 4H, 2CH₂).

MS-LC: MH+=292.13; r.t.=7.11 min.

### Example 20

### 2-methyl-5-[(2-piperidin-1-ylethyl)amino]-1,3-benzothiazole-4,7-dione

MS-LC: MH+=306.24; r.t.=7.22 min.

### Example 21

### 5-{[2-(diisopropylamino)ethyl]amino}-2-methyl-1,3-benzothiazole-4,7-dione

MS-LC: MH+=322.33; r.t.=7.37 min.

### Example 22

### 5-[(1-benzylpyrrolidin-3-yl)amino]-2-methyl-1,3-benzothiazole-4,7-dione

MS-LC: MH+=354.28; r.t.=7.70 min.

### Example 23

### 5-{[3-(dimethylamino)propyl]amino}-2-methyl-1,3-benzothiazole-4,7-dione

MS-LC: MH+=280.15; r.t.=7.01 min.

### Example 24

### 2-methyl-5-{[2-(1-methylpyrrolidin-2-yl)ethyl]amino}-1,3-benzothiazole-4,7-dione

MS-LC: MH+=306.30; r.t.=7.23 min.

### Example 25

### 2-methyl-5-{[3-(2-methylpiperidin-1-yl)propyl]amino}-1,3-benzothiazole-4,7-dione

MS-LC: MH+=334.29; r.t.=7.38 min.

### Example 26

### 5-{[4-(dimethylamino)butyl]amino}-2-methyl-1,3-benzothiazole-4,7-dione

MS-LC: MH+=294.16; r.t.=7.11 min.

### Example 27

### 5-{[5-(dimethylamino)pentyl]amino}-2-methyl-1,3-benzothiazole-4,7-dione

MS-LC: MH+=308.16; r.t.=7.22 min.

### Example 28

### 5-(2,3-dihydro-1H-inden-1-ylamino)-2-methyl-1,3-benzothiazole-4,7-dione

MS-LC: MH+=311.26; r.t.=10.16 min.

### Example 29

### 5-{benzyl[2-(dimethylamino)ethyl]amino}-2-methyl-1,3-benzothiazole-4,7-dione

NMR ¹H (DMSO d6, 400 MHz, δ): 7.37-7.28 (m, 5H, H arom.); 5.61 (s, 1H, CH); 4.57 (s, 2H, CH₂); 3.71-3.68 (m, 2H, CH₂); 2.75 (s, 3H, CH₃); 2.39-2.37 (m, 2H, CH₂); 1.95 (s, 6H, 2 CH₃).

MS-LC: MH+=365.10; r.t.=7.70 min.

### Example 30

### tert-butyl methyl{3-[(2-methyl-4,7-dioxo-4,7-dihydro-1,3-benzothiazol-5-yl)amino]propyl}carbamate

NMR ¹H (DMSO d6, 400 MHz, δ): 7.75 (m, 1H, NH); 5.45 (s, 1H, CH); 3.22-3.18 (m, 2H, CH₂); 3.15-3.12 (m, 2H, CH₂); 2.76 (m, 3H, CH₃); 2.75 (s, 3H, CH₃); 1.78-1.75 (m, 2H, CH₂); 1.35 (m, 9H, 3 CH₃).

MS-LC: MH+=366.15; r.t.=9.61 min.

### Example 31

### tert-butyl 3-[(2-methyl-4,7-dioxo-4,7-dihydro-1,3-benzothiazol-5-yl)amino]propylcarbamate

MS-LC: MH+=352.22; r.t.=9.09 min.

### Example 32

### 2-methyl-5-{[3-(methylamino)propyl]amino}-1,3-benzothiazole-4,7-dione hydrochloride

25 mg (68.5 μmol) of the compound of Example 30 is suspended in 10 ml of diethylether. 4 ml of a molar solution of hydrochloric acid in ether is added, then the reaction mixture is stirred at ambient temperature for 2 hours. The resulting precipitate is collected by filtration, followed by washing with ether then drying under reduced pressure in order to produce a brownish-red solid.

NMR ¹H (DMSO d6, 400 MHz, δ): 8.61 (m, 2H, NH₂⁺); 7.84-7.81 (m, 1H, NH); 5.55 (s, 1H, CH); 3.29-3.24 (m, 2H, CH₂); 2.91-2.88 (m, 2H, CH₂); 2.75 (s, 3H, CH₃); 2.53-2.52 (m, 3H, CH₃); 1.89-1.86 (m, 2H, CH₂).

MS-LC: MH+=266.06; r.t.=7.04 min.

### Example 33

### 5-[(3-aminopropyl)amino]-2-methyl-1,3-benzothiazole-4,7-dione

20 mg (57 μmol) of the compound of Example 30 is suspended in 10 ml of diethylether. 840 μl of a molar solution of hydrochloric acid in ether is added then the reaction mixture is stirred at ambient temperature for 2 hours. The resulting precipitate is collected by filtration, followed by washing with ether then drying under reduced pressure in order to produce a brownish-red solid.

NMR ¹H (DMSO d6, 400 MHz, δ): 7.84-7.78 (m, 3H, NH, NH₂); 5.56 (s, 1H, CH); 3.28-3.23 (m, 2H, CH₂); 2.86-2.81 (m, 2H, CH₂); 2.75 (s, 3H, CH₃); 1.85-1.82 (m, 2H, CH₂).

MS-LC: MH+=280.15; r.t.=7.01 min.

### Example 34

### 6-chloro-5-{[2-(dimethylamino)ethyl]amino}-2-methyl-1,3-benzothiazole-4,7-dione

58.6 mg (0.22 mmol) of intermediate 2.1 is placed in solution in 5 ml of acetic acid. 32.5 mg (0.24 mmol; 1.1 eq.) of N-chlorosuccinimide is added and the reaction mixture is stirred for 3 hours at ambient temperature. After concentration, the residue is purified by chromatography on a silica column (eluent: dichloromethane/methanol 90/10) and the expected product is obtained, after taking up in ethyl ether, in the form of a violet-coloured powder.

NMR ¹H (DMSO d6, 400 MHz, δ): 7.31 (m, 1H, NH); 3.79-3.74 (m, 2H, CH₂); 2.75 (s, 3H, CH₃); 2.47-2.44 (m, 2H, CH₂); 2.13 (s, 6H, 2 CH₃).

MS-LC: MH+=300.09; r.t.=7.17 min.

### Example 35

### 6-bromo-5-{[2-(dimethylamino)ethyl]amino}-2-methyl-1,3-benzothiazole-4,7-dione

102 mg (0.38 mmol) of intermediate 2.1 is placed in solution in 10 ml of acetic acid. 77.3 mg (0.43 mmol; 1.1 eq.) of N-bromosuccinimide is added and the reaction mixture is stirred for 3 hours at ambient temperature. After concentration under reduced pressure, the residue is purified by chromatography on a silica column (eluent: dichloromethane/methanol 90/10) and the expected product is obtained, after taking up in ethyl ether, in the form of a violet-coloured powder.

NMR ¹H (DMSO d6, 400 MHz, δ): 7.24 (m, 1H, NH); 3.78-3.74 (m, 2H, CH₂); 2.75 (s, 3H, CH₃); 2.45-2.42 (m, 2H, CH₂); 2.11 (s, 6H, 2 CH₃).

MS-LC: MH+=343.97; r.t.=7.22 min.

### Example 36

### 6-(butylthio)-5-{[2-(dimethylamino)ethyl]amino}-2-methyl-1,3-benzothiazole-4,7-dione

20 μl (0.115 mmol; 1.2 eq.) of diisopropylethylamine and 16 μl (0.154 mmol; 1.6 eq.) of butanethiol are added to 33 mg (96 μmol) of the compound of Example 35 in solution in 4 ml of anhydrous ethanol. The reaction mixture is maintained under stirring for 24 hours at 60° C., then after concentration under reduced pressure, the residue is purified by chromatography on a silica column (eluent: dichloromethane/methanol 95/5) and the expected product is obtained, after taking up in ethyl ether, in the form of a violet-coloured powder.

NMR ¹H (DMSO d6, 400 MHz, δ): 7.56 (m, 1H, NH); 3.84-3.83 (m, 2H, CH₂); 2.75 (s, 3H, CH₃); 2.64-2.60 (t, 2H, CH₂); 2.45-2.42 (m, 2H, CH₂); 2.20 (s, 6H, 2 CH₃); 1.44-1.46 (m, 2H, CH₂); 1.37-1.33 (m, 2H, CH₂); 0.85-0.82 (t, 3H, CH₃).

### Example 37

### 5-{[2-(dimethylamino)ethyl]amino}-2-(morpholin-4-ylmethyl)-1,3-benzothiazole-4,7-dione

### 37.1) 2-(bromomethyl)-5-methoxy-1,3-benzothiazole

2.58 g (14.5 mmol; 1.3 eq.) of N-bromosuccinimide and a spatula tip's worth of aza-bis-isobutyronitrile are added to 2 g (11.16 mmol) of 2-methyl-5-methoxy-1,3-benzothiazole in solution in 25 ml of anhydrous carbon tetrachloride. The reaction mixture is heated under reflux and under irradiation for 6 hours, with a spatula tip's worth of aza-bis-isobutyronitrile added every 2 hours. After returning to ambient temperature, the insoluble part formed is filtered, the solvent is evaporated off under reduced pressure and the residue is purified by chromatography on a silica column (eluent: ethyl acetate/heptane 1/4). The expected product is obtained in the form of a white solid.

NMR ¹H (DMSO d6, 400 MHz, δ): 7.98-7.96 (m, 1H, H arom.); 7.54-7.53 (m, 1H,H arom.); 7.13-7.10 (m, 1H, H arom.); 5.09 (s, 2H, CH₂); 3.84 (s, 3H, CH₃).

MS-LC: MH+=258.38; r.t.=10.36 min.

### 37.2) 5-methoxy-2-(morpholin-4-ylmethyl)-1,3-benzothiazole

678 μl of diisopropylethylamine (3.9 mmol; 2 eq.) is added to 0.5 g of intermediate 37.1 in solution in 20 ml of anhydrous toluene. 187 μl (2.14 mmol; 1.1 eq.) of morpholine and a spatula tip's worth of sodium iodide are added to the previous solution, then the reaction mixture is maintained under stirring at 80° C. for 3 hours. After cooling down, the reaction medium is washed with water (3 times 20 ml), then the organic phase is dried over magnesium sulphate and concentrated. Purification by chromatography on a silica column (eluent: ethyl acetate/heptane 1/1) allows the expected product to be obtained in the form of a beige solid.

NMR ¹H (DMSO d6, 400 MHz, δ): 7.91-7.89 (m, 1H, H arom.); 7.47-7.46 (m, 1H, H arom.); 7.05-7.02 (m, 1H, H arom.); 3.92 (s, 2H, CH₂); 3.82 (s, 3H, CH₃); 3.63-3.61 (m, 4H, 2CH₂); 2.56-2.53 (m, 4H, 2CH₂).

MS-LC: MH+=265.10; r.t.=7.55 min.

### 37.3) 5-methoxy-2-(morpholin-4-ylmethyl)-4-nitro-1,3-benzothiazole

84 mg (0.83 mmol; 1.2 eq.) of potassium nitrate is added by portions to a solution at 0° C. of 0.2 g (0.76 mmol) of intermediate 37.2 in 0.7 ml of concentrated sulphuric acid. After returning to ambient temperature, the reaction mixture is stirred for 18 hours, neutralized by adding a 10M aqueous solution of soda followed by extracting with 3 times 50 ml dichloromethane. The resulting organic phase is dried over magnesium sulphate followed by concentrating, then purifying by chromatography on a silica column (eluent: ethyl acetate/heptane 1/1). The expected product is obtained in the form of a yellow oil.

NMR ¹H (DMSO d6, 400 MHz, δ): 8.26-8.24 (m, 1H, H arom.); 7.48-7.46 (m, 1H, H arom.); 3.98-3.96 (2s, 5H, CH₃, CH₂); 3.63-3.61 (m, 4H, 2CH₂); 2.59-2.56 (m, 4H, 2 CH₂).

MS-LC: MH+=310.11; r.t.=8.03 min.

### 37.4) 5-methoxy-2-(morpholin-4-ylmethyl)-1,3-benzothiazol-4-amine

0.93 g (4.11 mmol; 5 eq.) of tin chloride is added to a solution of 0.254 g (0.822 mmol) of intermediate 37.3 in 7 ml of concentrated hydrochloric acid. The reaction mixture is maintained under stirring for 3 hours at 70° C. After returning to ambient temperature, the medium is diluted by adding 20 ml of ethyl acetate, followed by neutralizing with a saturated solution of NaHCO₃ and finally washing with 3 times 20 ml of water. The organic phases are combined, followed by drying over magnesium sulphate and concentrating in order to provide the expected product in the form of a beige powder.

NMR ¹H (DMSO d6, 400 MHz, δ): 7.12-7.10 (m, 1H, arom H.); 7.02-7.00 (m, 1H, arom H.); 5.04 (s, 2H, NH₂); 3.88 (s, 2H, CH₂); 3.81 (s, 3H, CH₃); 3.63-3.60 (m, 4H, 2 CH₂); 2.55-2.52 (m, 4H, 2CH₂).

MS-LC: MH+=280.11; r.t.=7.29 min.

### 37.5) 5-methoxy-2-(morpholin-4-ylmethyl)-1,3-benzothiazole-4,7-dione

A solution of 84 mg (0.31 mmol; 1.8 eq.) of Fremy's salt, dissolved in 14 ml of a buffer solution (0.3M) of sodium hydrogen phosphate, is added to 0.0483 mg (0.17 mmol) of intermediate 37.4 in solution in 10 ml of acetone. The reaction mixture is stirred for 18 hours at ambient temperature, followed by extracting with 3 times 30 ml of dichloromethane, the organic phases then being washed with twice 20 ml of water. The organic phases are then combined, followed by drying over magnesium sulphate and then concentrating under reduced pressure. The residue is purified by chromatography on a silica column (eluent: ethyl acetate/heptane 1/1) and the expected product is obtained in the form of a yellow oil.

MS-LC: MH+=295.06; r.t.=7.11 min.

### 37.6) 5-{[2-(dimethylamino)ethyl]amino}-2-(morpholin-4-ylmethyl)-1,3-benzothiazole-4,7-dione

The experimental protocol used is identical to that described for Example 1, intermediate 37.5 replacing 5-methoxy-2-methyl-4,7-dioxobenzothiazole.

MS-LC: MH+=351.38; r.t.=3.07 min.

### Example 38

### 5-{[2-(dimethylamino)ethyl]amino}-2-[(4-phenylpiperazin-1-yl)methyl]-1,3-benzothiazole-4,7-dione

The experimental protocol used is identical to that described for Example 37, N-phenylpiperazine replacing morpholine in the second stage.

MS-LC: MH+=426.18; r.t.=7.39 min.

### Example 39

### 5-{[2-(dimethylamino)ethyl]amino}-2-(piperidin-1-ylmethyl)--1,3-benzothiazole-4,7-dione

The experimental protocol used is identical to that described for Example 37, piperidine replacing morpholine in the second stage.

MS-LC: MH+=349.13; r.t.=2.82 min.

The compounds of Examples 40 to 52 are obtained in a similar manner to that described for Example 15, suitable primary or secondary amines replacing aniline in the fourth and last stage.

### Example 40

### 5-{[2-(dimethylamino)ethyl]amino}-2-ethyl-1,3-benzoxazole-4,7-dione or 6-{[2-(dimethylamino)ethyl]amino}-2-ethyl-1,3-benzoxazole-4,7-dione

Melting point: 123° C.

NMR ¹H (DMSO d6, 400 MHz, δ): 7.39 (t, 1H, NH); 5.30 (s, 1H, CH); 3.30-3.31 (m, 2H, CH₂); 3.24-3.20 (m, 2H, CH₂); 2.95-2.88 (q, 2H, CH₂); 2.17 (s, 6H, 2 CH₃); 1.30 (t, 3H, CH₃).

MS-LC: MH+=264.13; r.t.=7.02 min.

### Example 41

### tert-butyl 2-[(2-ethyl-4,7-dioxo-4,7-dihydro-1,3-benzoxazol-5-yl)(methyl)amino]ethylcarbamate or tert-butyl 2-[(2-ethyl-4,7-dioxo-4,7-dihydro-1,3-benzoxazol-6-yl)(methyl)amino]ethylcarbamate

Melting point: 135° C.

NMR ¹H (DMSO d6, 400 MHz, δ): 7.82 (t, 1H, NH); 5.36 (s, 1H, CH); 3.38-3.36 (m, 2H, CH₂); 3.30-3.27 (m, 2H, CH₂); 2.93-2.88 (q, 2H, CH₂); 2.79 (s, 3H, CH₃); 1.37-1.26 (m, 12H, 4 CH₃).

MS-LC: MH+=350.14; r.t.=9.72 min.

### Example 42

### tert-butyl 2-[(2-ethyl-4,7-dioxo-4,7-dihydro-1,3-benzoxazol-5-yl)amino]ethylcarbamate or tert-butyl 2-[(2-ethyl-4,7-dioxo-4,7-dihydro-1,3-benzoxazol-6-yl)amino]ethylcarbamate

Melting point: 173° C.

NMR ¹H (DMSO d6, 400 MHz, δ): 7.73 (t, 1H, NH); 6.97 (t, 1H, NH); 5.36 (s, 1H, CH); 3.20-3.17 (m, 2H, CH₂); 3.15-3.12 (m, 2H, CH₂); 2.93-2.88 (q, 2H, CH₂); 1.36 (s, 9H, 3CH₃); 1.28 (t, 3H, CH₃).

MS-LC: MH+=336.23; r.t.=9.24 min.

### Example 43

### 5-{[3-(dimethylamino)propyl]amino}-2-ethyl-1,3-benzoxazole-4,7-dione or 6-{[3-(dimethylamino)propyl]amino}-2-ethyl-1,3-benzoxazole-4,7-dione

Melting point: 101° C.

NMR ¹H (DMSO d6, 400 MHz, δ): 8.09 (t, 1H, NH); 5.28 (s, 1H, CH); 3.21-3.16 (m, 2H, CH₂); 2.93-2.88 (q, 2H, CH₂); 2.28-2.25 (m, 2H, CH₂); 2.13 (s, 6H, 2 CH₃); 1.71-1.67 (m, 2H, CH₂); 1.28 (t, 3H, CH₃).

MS-LC: MH+=278.19; r.t.=7.09 min.

### Example 44

### 2-ethyl-5-{[2-(1-methylpyrrolidin-2-yl)ethyl]amino}-1,3-benzoxazole-4,7-dione or 2-ethyl-6-{[2-(1-methylpyrrolidin-2-yl)ethyl]amino}-1,3-benzoxazole-4,7-dione

Melting point: 121° C.

NMR ¹H (DMSO d6, 400 MHz, δ): 8.11 (t, 1H, NH); 5.24 (s, 1H, CH); 3.19-3.17 (m, 2H, CH₂); 2.95-2.93 (m, 1H, CH); 2.92-2.87 (q, 2H, CH₂); 2.21 (s, 3H, CH₃); 2.16-2.05 (m, 2H, CH₂); 1.88-1.84 (m, 2H, CH₂); 1.63-1.57 (m, 4H, 2 CH₂); 1.28 (t, 3H, CH₃).

MS-LC: MH+=304.20; r.t.=7.20 min.

### Example 45

### 5-{[4-(dimethylamino)butyl]amino}-2-ethyl-1,3-benzoxazole-4,7-dione or 6-{[4-(dimethylamino)butyl]amino}-2-ethyl-1,3-benzoxazole-4,7-dione

NMR ¹H (DMSO d6, 400 MHz, δ): 8.06 (t, 1H, NH); 5.28 (s, 1H, CH); 3.17-3.12 (m, 2H, CH₂); 2.93-2.88 (q, 2H, CH₂); 2.22-2.19 (m, 2H, CH₂); 2.11 (s, 6H, 2CH₃); 1.61-1.56 (m, 2H, CH₂); 1.46-1.42 (m, 2H, CH₂); 1.28 (t, 3H, CH₃) MS-LC: MH+=292.20; r.t.=7.10 min.

### Example 46

### 2-ethyl-5-[(4-pyrrolidin-1-ylbutyl)amino]-1,3-benzoxazole-4,7-dione or 2-ethyl-6-[(4-pyrrolidin-1-ylbutyl)amino]-1,3-benzoxazole-4,7-dione

Melting point: 102° C.

NMR ¹H (DMSO d6, 400 MHz, δ): 7.95 (t, 1H, NH); 5.28 (s, 1H, CH); 3.17-3.13 (m, 2H, CH₂); 2.93-2.87 (q, 2H, CH₂); 2.41-2.37 (m, 6H, 3CH₂); 1.63-1.58 (m, 2H, CH₂); 1.49-1.45 (m, 2H, CH₂); 1.28 (t, 3H, CH₃).

MS-LC: MH+=318.20; r.t.=7.30 min.

### Example 47

### 5-{[5-(dimethylamino)pentyl]amino}-2-ethyl-1,3-benzoxazole-4,7-dione or 6-{[5-(dimethylamino)pentyl]amino}-2-ethyl-1,3-benzoxazole-4,7-dione

NMR ¹H (DMSO d6, 400 MHz, δ): 7.83 (t, 1H, NH); 5.27 (s, 1H, CH); 3.17-3.13 (m, 2H, CH₂); 2.93-2.87 (q, 2H, CH₂); 2.18-2.14 (m, 2H, CH₂); 2.09 (s, 6H, 2CH₃); 1.58-1.54 (m, 2H, CH₂); 1.41-1.38 (m, 2H, CH₂); 1.28 (t, 3H, CH₃).

MS-LC: MH+=306.20; r.t.=7.30 min.

### Example 48

### Mixture of 5-{[6-(dimethylamino)hexyl]amino}-2-ethyl-1,3-benzoxazole-4,7-dione and 6-{[6-(dimethylamino)hexyl]amino}-2-ethyl-1,3-benzoxazole-4,7-dione

MS-LC: MH+=320.20; r.t.=7.50 min.

### Example 49

### Mixture of 2-ethyl-5-(4-methylpiperazin-1-yl)-1,3-benzoxazole-4,7-dione and 2-ethyl-6-(4-methylpiperazin-1-yl)-1,3-benzoxazole-4,7-dione

MS-LC: MH+=276.10; r.t.=7.10 min.

### Example 50

### Mixture of 2-ethyl-5-[(1-ethylhexyl)amino]-1,3-benzoxazole-4,7-dione and 2-ethyl-6-[(1-ethylhexyl)amino]-1,3-benzoxazole-4,7-dione

MS-LC: MH+=305.20; r.t.=11.50 min.

### Example 51

### Mixture of 5-azocan-1-yl-2-ethyl-1,3-benzoxazole-4,7-dione and 6-azocan-1-yl-2-ethyl-1,3-benzoxazole-4,7-dione

MS-LC: MH+=289.20; r.t.=10.40 min.

### Example 52

### Mixture of 2-ethyl-5-morpholin-4-yl-1,3-benzoxazole-4,7-dione and 2-ethyl-6-morpholin-4-yl-1,3-benzoxazole-4,7-dione

MS-LC: MH+=263.10; r.t.=−8.60 min.

### Example 53

### 6-chloro-5-{[2-(dimethylamino)ethyl]amino}-2-ethyl-1,3-benzoxazole-4,7-dione or 5-chloro-6-{[2-(dimethylamino)ethyl]amino}-2-ethyl-1,3-benzoxazole-4,7-dione

The experimental protocol used is identical to that described for Example 34, the compound of Example 40 replacing intermediate 2.1. Melting point: 110° C.

NMR ¹H (DMSO d6, 400 MHz, δ): 7.35 (t, 1H, NH); 3.78-3.74 (m, 2H, CH₂); 2.94-2.89 (q, 2H, CH₂); 2.48-2.45 (m, 2H, CH₂); 2.15 (s, 6H, 2CH₃); 1.28 (t, 3H, CH₃).

MS-LC: MH+=298.10; r.t.=7.20 min.

### Example 54

### 6-bromo-5-{[2-(dimethylamino)ethyl]amino}-2-ethyl-1,3-benzoxazole-4,7-dione or 5-bromo-6-{[2-(dimethylamino)ethyl]amino}-2-ethyl-1,3-benzoxazole-4,7-dione

The experimental protocol used is identical to that described for Example 35, the compound of Example 40 replacing intermediate 2.1.

NMR ¹H (DMSO d6, 400 MHz, δ): 7.27 (t, 1H, NH); 3.78-3.74 (m, 2H, CH₂); 2.94-2.89 (q, 2H, CH₂); 2.46-2.43 (m, 2H, CH₂); 2.13 (s, 6H, 2 CH₃); 1.26 (t, 3H, CH₃).

MS-LC: MH+=342.00; r.t.=7.30 min.

### Example 55

### 5-{[2-(dimethylamino)ethyl]amino}-2-ethyl-6-methyl-1,3-benzoxazole-4,7-dione

### 55.1) 2-diazo-5-methylcyclohexane-1,3-dione

12.25 ml (87.2 mmol; 2.2 eq.) of triethylamine and 8.57 g (35.67 mmol; 0.9 eq.) of 4-acetamidobenzenesulphonylazide are added to a solution of 5 g (39.6 mmol) of 5-methylcyclohexane-1,3-dione in 100 ml of dichloromethane. The reaction mixture is stirred for 75 minutes at ambient temperature, then cooled down to 0° C. and filtered on a silica bed. After concentration under reduced pressure, the solution is washed with 3 times 50 ml of water. The organic phases are combined, dried over sodium sulphate and concentrated. The resulting solid is taken up in ethyl ether followed by filtering and drying under reduced pressure. It is used in the following stage without other purification.

MS-LC: MH+=153.49; r.t.=7.21 min.

### 55.2) 2-ethyl-6-methyl-6,7-dihydro-1,3-benzoxazol-4(5H)-one

285 mg (0.644 mmol; 0.02 eq.) of rhodium acetate is added to a solution of 4.9 g (32.2 mmol) of intermediate 55.1 in 50 ml of propionitrile. The reaction mixture is maintained under stirring under an inert argon atmosphere at 60° C. for 2 hours. The solvent is then evaporated off and the residue is purified by chromatography on a silica column (eluent: ethyl acetate/heptane 1/1). The expected product is obtained in the form of a yellow oil.

NMR 1H (DMSO d6, 400 MHz, δ): 3.02-2.97 (m, 1H, CH); 2.80-2.74 (q, 2H, CH₂); 2.68-2.61 (m, 1H, CH₂); 2.44-2.39 (m, 2H, CH₂); 2.34-2.30 (m, 1H, CH₂); 1.23 (t, 3H, CH₃); 1.08 (s, 3H, CH₃).

MS-LC: MH+=180.25; r.t.=8.55 min.

### 55.3) (4E)-2-ethyl-6-methyl-6,7-dihydro-1,3-benzoxazol-4(5H)-one oxime

647 mg (9.31 mmol; 1.2 eq.) of hydroxylamine hydrochloride and 764 mg (9.31 mmol; 1.2 eq.) of ammonium acetate are added to a solution of 1.39 g (7.76 mmol) of intermediate 55.2 in 200 ml of methanol. The reaction mixture is stirred for 90 minutes under reflux of the methanol, then the solvent is evaporated off, the residue is taken up in 50 ml of water then neutralized using a saturated solution of NaHCO₃. The expected product is extracted twice with 50 ml of ethyl acetate then washed twice with 30 ml of water. The organic phases are combined, dried over sodium sulphate and concentrated under reduced pressure. The desired product is obtained in the form of a dark yellow solid, used without other purification in the following stage.

MS-LC: MH+=195.09; r.t.=8.73 min.

### 55.4) 2-ethyl-6-methyl-1,3-benzoxazol-4-amine

1.45 g (7.46 mmol) of intermediate 55.3 is dissolved in 25 g of polyphosphoric acid. After stirring for 1 hour at 140° C., the solution is hydrolyzed by the addition of iced water, then neutralized by a 50% aqueous solution of soda. The product obtained is extracted with dichloromethane, and the organic phase is washed 3 times with 25 ml of water, dried over sodium sulphate and concentrated under reduced pressure. The desired product is obtained after purification by chromatography on a silica column (eluent: dichloromethane/ethanol 98/2).

MS-LC: MH+=177.21; r.t.=9.12 min.

### 55.5) 2-ethyl-6-methyl-1,3-benzoxazole-4,7-dione

The experimental protocol used is identical to that described for Stage 15.3 of Example 15, intermediate 55.4 replacing intermediate 15.2.

NMR ¹H (DMSO d6, 400 MHz, δ): 6.72 (s, 1H, CH); 2.98-2.93 (q, 2H, CH₂); 2.04 (s, 3H, CH₃); 1.30 (t, 3H, CH₃).

MS-LC: MH+=192.06; r.t.=8.93 min.

### 55.6) 5-{[2-(dimethylamino)ethyl]amino}-2-ethyl-6-methyl-1,3-benzoxazole-4,7-dione

The experimental protocol used is identical to that described for Stage 15.4 of Example 15, intermediate 55.5 replacing intermediate 15.3 and N,N-dimethylethylenediamine replacing aniline. Melting point: 135° C.

NMR ¹H (DMSO d6, 400 MHz, δ): 6.63 (t, 1H, NH); 3.62-3.58 (m, 2H, CH₂); 2.92-2.86 (q, 2H, CH₂); 2.44-2.41 (m, 2H, CH₂); 2.14 (s, 6H, 2 CH₃); 1.97 (s, 3H, CH₃); 1.27 (t, 3H, CH₃).

MS-LC: MH+=278.12; r.t.=7.27 min.

### Example 56

### 2-cyclopropyl-5-{[2-(dimethylamino)ethyl]amino}-1,3-benzoxazole-4,7-dione or 2-cyclopropyl-6-{[2-(dimethylamino)ethyl]amino}-1,3-benzoxazole-4,7-dione

The experimental protocol used is identical to that described for Example 55, cyclohexane-1,3-dione replacing 5-methylcyclohexane-1,3-dione in the first stage and cyclopropanecarbonitrile replacing propionitrile in the second stage. Melting point: 155° C.

NMR ¹H (DMSO d6, 400 MHz, δ): 7.35 (t, 1H, NH); 5.27 (s, 1H, CH); 3.30-3.18 (m, 2H, CH₂); 2.49-2.46 (m, 2H, CH₂); 2.28-2.25 (m, 1H, CH); 2.17 (s, 6H, 2 CH₃); 1.18-1.07 (m, 4H, 2 CH₂).

MS-LC: MH+=276.10; r.t.=7.10 min.

### Example 57

### Mixture of 5-{[2-(dimethylamino)ethyl]amino}-2-phenyl-1,3-benzoxazole-4,7-dione and 6-{[2-(dimethylamino)ethyl]amino}-2-phenyl-1,3-benzoxazole-4,7-dione

The experimental protocol used is identical to that described for Example 15, trimethyl orthobenzoate replacing triethyl orthopropionate in the first stage and N,N-dimethylethylenediamine replacing aniline in the fourth and last stage. Melting point: 147° C.

NMR ¹H (DMSO d6, 400 MHz, δ): 8.15-8.08 (m, 2H, H arom); 7.70-7.61 (m, 3H, H arom.); 7.33 (t, 1H, NH); 5.38 (s, 1H, CH); 3.26-3.21 (m, 4H, 2 CH₂); 2.19 (s, 6H, 2 CH₃).

The two components of the mixture can be characterized by the NMR shifts (400 MHz) of the single proton of the benzoxazoledione ring which are 5.38 and 5.39 ppm.

MS-LC: MH+=312.20; r.t.=7.70 min.

### Example 58

### Mixture of 5-{[6-(dimethylamino)hexyl]amino}-2-phenyl-1,3-benzoxazole-4,7-dione and 6-{[6-(dimethylamino)hexyl]amino}-2-phenyl-1,3-benzoxazole-4,7-dione

The experimental protocol used is identical to that described for Example 15, trimethyl orthobenzoate replacing triethyl orthopropionate in the first stage and 6-(dimethylamino)hexylamine replacing aniline in the fourth and last stage. The two components of the mixture can be characterized by the NMR shifts (400 MHz) of the single proton of the benzoxazoledione ring which are 5.34 and 5.35 ppm.

MS-LC: MH+=368.20; r.t.=8.10 min.

### Example 59

### 5-[(1-ethylhexyl)amino]-2-phenyl-1,3-benzoxazole-4,7-dione or 6-[(1-ethylhexyl)amino]-2-phenyl-1,3-benzoxazole-4,7-dione

The experimental protocol used is identical to that described for Example 15, trimethyl orthobenzoate replacing triethyl orthopropionate in the first stage and 2-ethylhexylamine replacing aniline in the fourth and last stage.

MS-LC: MH+=353.20; r.t.=12.50 min.

### Example 60

### Mixture of 2-(2,6-difluorophenyl)-5-[(2-pyrrolidin-1-ylethyl)amino]-1,3-benzoxazole-4,7-dione and 2-(2,6-difluorophenyl)-6-[(2-pyrrolidin-1-ylethyl)amino]-1,3-benzoxazole-4,7-dione

### 60.1) 2-(2,6-difluorophenyl)-4-nitro-1,3-benzoxazole

2 g (32.4 mmol; 1 eq.) of boric acid is added to a solution of 5 g (32.4 mmol) of 2-amino-3-nitrophenol and 5.12 g (32.4 mmol; 1 eq.) of 2,6-difluorobenzoic acid in 50 ml of xylene. The mixture is heated under reflux of the xylene for 8 hours with elimination of the water formed by a Dean-Stark apparatus. After returning to ambient temperature, the reaction medium is diluted by 100 ml of ethyl acetate and neutralized by a 10% aqueous solution of soda. The organic phase is washed 3 times with 50 ml of water then with a saturated solution of NaCl before being dried over sodium sulphate, filtered and concentrated under reduced pressure. The 2-(2,6-difluorophenyl)-4-nitro-1,3-benzoxazole is used without other purification in the following stage.

MS-LC: MH+=277.00; r.t.=10.45 min.

### 60.2) 2-(2,6-difluorophenyl)-1,3-benzoxazol-4-amine

14.3 g (63.5 mmol; 5 eq.) of tin chloride is added to a solution of 3.5 g (12.7 mmol) of 2-(2,6-difluorophenyl)-4-nitro-1,3-benzoxazole in 60 ml of concentrated hydrochloric acid. The mixture is stirred for 2 hours at 60° C., then, after returning to ambient temperature and the addition of 100 ml of water, is neutralized by a 50% aqueous solution of soda. The precipitate formed is filtered on a Celite bed and washed with ethanol. The resulting solution is concentrated under reduced pressure, then the desired product is extracted 3 times with 50 ml of ethyl acetate. The organic phases are combined, washed twice with 30 ml of a saturated solution of sodium chloride, dried over sodium sulphate and concentrated under reduced pressure. The 2-(2,6-difluorophenyl)-1,3-benzoxazol-4-amine is used without other purification in the following stage.

MS-LC: MH+=247.08; r.t.=10.02 min.

### 60.3) 2-(2,6-difluorophenyl)-1,3-benzoxazole-4,7-dione

The experimental protocol used is identical to that described for Stage 15.3 of Example 15, intermediate 60.2 replacing intermediate 15.2. The expected product is obtained in the form of yellow crystals.

MS-LC: MH+=261.93; r.t.=9.62 min.

### 60.4) mixture of 2-(2,6-difluorophenyl)-5-[(2-pyrrolidin-1-ylethyl)amino]-1,3-benzoxazole-4,7-dione and 2-(2,6-difluorophenyl)-6-[(2-pyrrolidin-1-ylethyl)amino]-1,3-benzoxazole-4,7-dione

The experimental protocol used is identical to that described for Stage 15.4 of Example 15, intermediate 60.3 replacing intermediate 15.3 and (2-aminoethyl)pyrrolidine replacing aniline. Melting point: 150° C.

NMR ¹H (DMSO d6, 400 MHz, δ): 7.78-7.76 (m, 1H, H arom.); 7.43-7.37 (m, 2H, H arom.); 5.41 (s, 1H, CH); 3.38-3.36 (m, 2H, CH₂); 3.28-3.26 (m, 4H, 2 CH₂); 2.68-2.64 (m, 2H, CH₂); 1.70-1.67 (m, 4H, 2 CH₂).

The two components of the mixture can be characterized by the NMR shifts (400 MHz) of the single proton of the benzoxazoledione ring which are 5.40 and 5.42 ppm.

MS-LC: MH+=373.99; r.t.=7.76 min.

The compounds of Examples 61 to 65 are obtained in similar manner to that described for Example 60.

### Example 61

### Mixture of 2-[4-(diethylamino)phenyl]-5-{[2-(dimethylamino)ethyl]amino}-1,3-benzoxazole-4,7-dione and 2-[4-(diethylamino)phenyl]-6-{[2-(dimethylamino)ethyl]amino}-1,3-benzoxazole-4,7-dione

NMR ¹H (DMSO d6, 400 MHz, δ): 7.91-7.89 (d, 2H, H arom.); 6.83-6.81 (d, 2H, H arom.); 5.29 (s, 1H, CH); 3.47-3.42 (m, 4H, 2 CH₂); 3.41-3.38 (m, 2H, CH₂); 3.25-3.21 (m, 2H, CH₂); 2.19 (s, 6H, 2 CH₃); 1.12 (t, 6H, 2 CH₃).

The two components of the mixture can be characterized by the NMR shifts (400 MHz) of the single proton of the benzoxazoledione ring which are 5.29 and 5.30 ppm.

MS-LC: MH+=383.20; r.t.=8.30 min.

### Example 62

### Mixture of 2-[4-(diethylamino)phenyl]-5-[(2-pyrrolidin-1-ylethyl)amino]-1,3-benzoxazole-4,7-dione and 2-[4-(diethylamino)phenyl]-6-[(2-pyrrolidin-1-ylethyl)amino]-1,3-benzoxazole-4,7-dione

NMR ¹H (DMSO d6, 400 MHz, δ): 7.91-7.88 (d, 2H, H arom.); 6.83-6.81 (d, 2H, H arom.); 5.29 (s, 1H, CH); 3.47-3.42 (m, 4H, 2 CH₂); 3.37-3.35 (m, 2H, CH₂); 3.26-3.23 (m, 4H, 2 CH₂); 2.66 (t, 2H, CH₂); 1.70-1.68 (m, 4H, 2 CH₂); 1.14 (t, 6H, 2 CH₃).

The two components of the mixture can be characterized by the NMR shifts (400 MHz) of the single proton of the benzoxazoledione ring which are 5.28 and 5.29 ppm.

MS-LC: MH+=409.10; r.t.=8.40 min.

### Example 63

### Mixture of 2-(4-chlorophenyl)-5-{[2-(dimethylamino)ethyl]amino}-1,3-benzoxazole-4,7-dione and 2-(4-chlorophenyl)-6-{[2-(dimethylamino)ethyl]amino}-1,3-benzoxazole-4,7-dione

Melting point: 169° C.

The two components of the mixture can be characterized by the NMR shifts (400 MHz) of the single proton of the benzoxazoledione ring which are 5.39 and 5.41 ppm.

MS-LC: MH+=346.20; r.t.=8.10 min.

### Example 64

### Mixture of 2-(4-chlorophenyl)-5-{[3-(dimethylamino)propyl]amino}-1,3-benzoxazole-4,7-dione and 2-(4-chlorophenyl)-6-{[3-(dimethylamino)propyl]amino}-1,3-benzoxazole-4,7-dione

MS-LC: MH+=360.10; r.t.=8.10 min.

### Example 65

### Mixture of 2-(4-chlorophenyl)-5-{[4-(dimethylamino)butyl]amino}-1,3-benzoxazole-4,7-dione and 2-(4-chlorophenyl)-6-{[4-(dimethylamino)butyl]amino}-1,3-benzoxazole-4,7-dione

NMR ¹H (DMSO d6, 400 MHz, δ): 8.13-8.09 (m, 2H, H arom.); 7.70-7.67 (m, 2H,H arom.); 5.36 (s, 1H, CH); 3.18-3.15 (m, 2H, CH₂); 2.25-2.21 (m, 2H, CH₂); 2.13 (s, 6H, 2 CH₃); 1.62-1.58 (m, 2H, CH₂); 1.48-1.44 (m, 2H, CH₂).

The two components of the mixture can be characterized by the NMR shifts (400 MHz) of the single proton of the benzoxazoledione ring which are 5.35 and 5.37 ppm.

MS-LC: MH+=374.10; r.t.=8.20 min.

### Example 66

### Mixture of 2-(2-fluorophenyl)-5-{[2-(dimethylamino)ethyl]amino}-1,3-benzoxazole-4,7-dione and 2-(2-fluorophenyl)-6-{[2-(dimethylamino)ethyl]amino}-1,3-benzoxazole-4,7-dione

### 66.1) 2-diazocyclohexane-1,3-dione

A mixture of 4-acetamidobenzenesulphonylazide (25 g, 104 mmol) and triethylamine (36 ml, 250 mmol) in dichloromethane maintained at a temperature below 30° C. by external cooling is treated dropwise by a solution of cyclohexane-1,3-dione (13 g, 115 mmol) in 200 ml of dichloromethane. The reaction mixture is stirred for 75 minutes at ambient temperature then filtered on Celite. After concentration to approximately 300 ml, the filtrate is washed with water then dried over sodium sulphate. The brown-yellow solid (14 g; 88%) obtained by evaporation of the solvent under reduced pressure is similar to that obtained in Example 55.1, and is used as it is in the following stage.

NMR ¹H (DMSO-d₆, δ): 1.93 (m, 2H); 2.50 (t, 4H).

NMR ¹³C (DMSO-d₆, δ): 18.20; 36.68; 190.96.

### 66.2) 2-(2-fluorophenyl)-6,7-dihydro-1,3-benzoxazol-4(5H)-one

A mixture of rhodium acetate (32 mg, 72 μmol) and 2-fluorobenzonitrile (2.31 ml; 22 mmol) in perfluorobenzene (5 ml) is treated at 60° C. dropwise by a solution of diazocyclohexanedione (obtained in Stage 66.1; 1 g; 7.24 mmol) in 5 ml of perfluorobenzene. The reaction medium is maintained at 60° C. until exhaustion of the release of nitrogen (1 hour; TLC on SiO₂: 2% MeOH/CH₂Cl₂). After cooling down to ambient temperature and filtration, the solvent of the filtrate is evaporated. The residue is purified by chromatography (SiO₂: AcOEt/heptane: 1/1) in order to produce a light yellow powder.

NMR ¹H(CDCl₃, δ): 2.31 (m, 2H); 2.66 (m, 2H,); 3.09 (t, 2H); 7.19-7.28 (m, 2H); 7.48-7.50 (m, 1H); 8.15-8.19 (m, 1H).

MS-LC: MH+=232.08; r.t.=9.28 min.

### 66.3) 5-bromo-2-(2-fluorophenyl)-6,7-dihydro-1,3-benzoxazol-4(5H)-one

A solution of intermediate 66.2 (470 mg, 2 mmol) in acetic acid (5 ml) is treated with bromine in acetic acid (0.2M; 10 ml; 2 mmol) for 4 days at ambient temperature (TLC on SiO₂: AcOEt/heptane:1/1). The reaction medium is then diluted with water and extracted using dichloromethane. The organic phases are combined, washed with a saturated solution of bicarbonate then with a 5% solution of sodium disulphite. After drying over sodium sulphate and elimination of the volatile constituents under reduced pressure, a yellow oil is obtained which is purified by chromatography (SiO₂: AcOEt/heptane:1/1) in order to produce a white powder.

NMR ¹H (DMSO-d₆, δ): 2.49 (m, 2H); 2.73 (m, 1H,); 3.15 (m, 2H); 4.95 (t, 1H,); 7.39-7.48 (m, 2H); 7.63-7.67 (m, 1H); 8.03-8.08 (t, 1H).

MS-LC: MH+=309.93; r.t.=10.08 min.

### 66.4) 2-(2-fluorophenyl)-4-hydroxy-1,3-benzoxazole

Intermediate 66.3 (6.52 g; 21 mmol) in solution in tetrahydrofuran (100 ml) is treated dropwise with diazabicyclo[5.4.0]undec-7-ene (4.7 ml; 31 mmol). When the reaction is complete (1.5 hours; TLC on SiO₂: AcOEt/heptane:1/1), the reaction mixture is diluted with ethyl acetate then washed successively with 1N hydrochloric acid and a saturated solution of sodium chloride. The combined organic phases are dried and concentrated in order to produce a brown residue which is purified by chromatography (SiO₂: AcOEt/heptane:1/1) in order to produce a beige powder.

NMR ¹H (DMSO-d₆, δ): 6.80 (d, 1H); 7.19-7.26 (m, 2H); 7.41-7.49 (m, 2H); 7.65 (m, 1H); 8.18 (t, 1H); 10.43 (s, 1H).

MS-LC: MH+=230.07; r.t.=10.03 min.

### 66.5) 2-(2-fluorophenyl)-1,3-benzoxazole-4,7-dione

The experimental protocol used is identical to that described for Stage 15.3 of Example 15, intermediate 66.4 replacing intermediate 15.2. A yellow powder is obtained.

NMR ¹H (DMSO-d₆, δ): 6.94 (broad, 2H); 7.45-7.54 (m, 2H); 7.74 (m, 2H); 8.18 (t, 1H).

MS-LC: MH+=244.04; r.t.=9.73 min. (61%) and MH₃+=246.06; r.t.=8.70 min.

### 66.6) Mixture of 2-(2-fluorophenyl)-5-{[2-(dimethylamino)ethyl]amino}-1,3-benzoxazole-4,7-dione and 2-(2-fluorophenyl)-6-{[2-(dimethylamino)ethyl]amino}-1,3-benzoxazole-4,7-dione

The experimental protocol used is identical to that described for Stage 15.4 of Example 15, intermediate 66.5 replacing intermediate 15.3 and N,N-dimethylethylenediamine replacing aniline. A ruby-coloured powder is obtained. Melting point: 191° C.

NMR ¹H (DMSO-d₆, δ): 2.19 (s, 6H); 2.5 (m, 2H); 3.27 (m, 2H); 5.41 (s, 1H); 7.42-7.52 (m, 3H); 7.70 (m, 2H); 8.13 (m, 1H).

The two components of the mixture can be characterized by the NMR shifts (400 MHz) of the single proton of the benzoxazoledione ring which are 5.40 and 5.41 ppm.

MS-LC: MH+=330.14; r.t.=7.69 min.

### Example 67

### Mixture of 2-(2-fluorophenyl)-5-[(2-pyrrolidin-1-ylethyl)amino]-1,3-benzoxazole-4,7-dione and 2-(2-fluorophenyl)-6-[(2-pyrrolidin-1-ylethyl)amino]-1,3-benzoxazole-4,7-dione

The experimental protocol used is identical to that described for Example 66, N-(2-aminoethyl)-pyrrolidine replacing N,N-dimethylethylenediamine. Melting point: 152° C.

The two components of the mixture can be characterized by the NMR shifts (400 MHz) of the single proton of the benzoxazoledione ring which are 5.39 and 5.41 ppm.

MS-LC: MH+=356.1; r.t.=7.8 min.

### Example 68

### Mixture of 2-(2-bromophenyl)-5-{[2-(dimethylamino)ethyl]amino}-1,3-benzoxazole-4,7-dione and 2-(2-bromophenyl)-6-{[2-(dimethylamino)ethyl]amino}-1,3-benzoxazole-4,7-dione

### 68.1) 2-(2-bromophenyl)-6,7-dihydro-1,3-benzoxazol-4(5H)-one

The experimental protocol used is identical to that described for Stage 66.2, 2-bromobenzonitrile replacing 2-fluorobenzonitrile. A yellow solid is obtained.

MS-LC: MH+=292.0; r.t.=9.8 min.

### 68.2) 5-bromo-2-(2-bromophenyl)-6,7-dihydro-1,3-benzoxazol-4(5H)-one

A mixture of intermediate 68.1 (6.6 g, 22 mmol) and CuBr₂ (10 g; 45 mmol) in ethyl acetate (250 ml) with approximately 1 ml of acetic acid added to it is taken to reflux for 3.5 hours (TLC on SiO₂: AcOEt/heptane:1/1). The reaction medium is then filtered on Celite, the filtrate is evaporated under reduced pressure and the residue is purified on a column (SiO₂: AcOEt/heptane:1/1) in order to produce a light yellow powder. MS-LC: MH+=371.8; r.t.=10.5 min.

### 68.3) 2-(2-bromophenyl)-5-{[2-(dimethylamino)ethyl]amino}-1,3-benzoxazole-4,7-dione

This compound is obtained from intermediate 68.2 according to the operating methods described for Stages 66.4, 66.5 and 66.6. Melting point: 138° C.

The two components of the mixture can be characterized by the NMR shifts (400 MHz) of the single proton of the benzoxazoledione ring which are 5.41 and 5.43 ppm.

MS-LC: MH+=390.0; r.t.=7.9 min.

### Example 69

### Mixture of 2-(2-bromophenyl)-5-[(2-pyrrolidin-1-ylethyl)amino]-1,3-benzoxazole-4,7-dione and 2-(2-bromophenyl)-6-[(2-pyrrolidin-1-ylethyl)amino]-1,3-benzoxazole-4,7-dione

The experimental protocol used is identical to that described for Example 68, N-(2-aminoethyl)-pyrrolidine replacing N,N-dimethylethylenediamine. Melting point: 122° C.

The two components of the mixture can be characterized by the NMR shifts (400 MHz) of the single proton of the benzoxazoledione ring which are 5.40 and 5.42 ppm.

MS-LC: MH+=416.0; r.t.=8.0 min.

### Example 70

### Mixture of 2-(2-bromophenyl)-5-{[3-(dimethylamino)propyl]amino}-1,3-benzoxazole-4,7-dione and 2-(2-bromophenyl)-6-{[3-(dimethylamino)propyl]amino}-1,3-benzoxazole-4,7-dione

The experimental protocol used is identical to that described for Example 68, N,N-dimethylpropylenediamine replacing N,N-dimethylethylenediamine. Melting point: 119° C.

The two components of the mixture can be characterized by the NMR shifts (400 MHz) of the single proton of the benzoxazoledione ring which are 5.38 and 5.40 ppm.

MS-LC: MH+=404.0; r.t.=8.0 min.

### Example 71

### Mixture of 2-(2-chlorophenyl}-5-{[2-(dimethylamino)ethyl]amino}-1,3-benzoxazole-4,7-dione and 2-(2-chlorophenyl)-6-{[2-(dimethylamino)ethyl]amino}-1,3-benzoxazole-4,7-dione

The experimental protocol used is identical to that described for Example 66, 2-chlorobenzonitrile replacing 2-fluorobenzonitrile. Melting point: 137° C.

The two components of the mixture can be characterized by the NMR shifts (400 MHz) of the single proton of the benzoxazoledione ring which are 5.39 and 5.41 ppm.

MS-LC: MH+=346.1; r.t.=7.8 min.

### Example 72

### Mixture of 2-(2-chlorophenyl)-5-[(2-pyrrolidin-1-ylethyl)amino]-1,3-benzoxazole-4,7-dione and 2-(2-chlorophenyl)-6-[(2-pyrrolidin-1-ylethyl)amino]-1,3-benzoxazole-4,7-dione

The experimental protocol used is identical to that described for Example 71, N-(2-aminoethyl)-pyrrolidine replacing N,N-dimethylethylenediamine. Melting point: 85° C.

The two components of the mixture can be characterized by the NMR shifts (400 MHz) of the single proton of the benzoxazoledione ring which are 5.40 and 5.41 ppm.

MS-LC: MH+=372.1; r.t.=8.0 min.

### Example 73

### Mixture of 2-(3-bromophenyl)-5-{[2-(dimethylamino)ethyl]amino}-1,3-benzoxazole-4,7-dione and 2-(3-bromophenyl)-6-{[2-(dimethylamino)ethyl]amino}-1,3-benzoxazole-4,7-dione

The experimental protocol used is identical to that described for Example 68, 3-bromobenzonitrile replacing 2-bromobenzonitrile. Melting point: 133° C.

The two components of the mixture can be characterized by the NMR shifts (400 MHz) of the single proton of the benzoxazoledione ring which are 5.39 and 5.41 ppm.

MS-LC: MH+=390.0; r.t.=8.1 min.

### Example 74

### Mixture of 2-(4-bromophenyl)-5-[(2-pyrrolidin-1-ylethyl)amino]-1,3-benzoxazole-4,7-dione and 2-(4-bromophenyl)-6-[(2-pyrrolidin-1-ylethyl)amino]-1,3-benzoxazole-4,7-dione

The experimental protocol used is identical to that described for Example 68, 4-bromobenzonitrile replacing 2-bromobenzonitrile, and N-(2-aminoethyl)-pyrrolidine replacing N,N-dimethylethylenediamine. Melting point: 181° C.

The two components of the mixture can be characterized by the NMR shifts (400 MHz) of the single proton of the benzoxazoledione ring which are 5.37 and 5.39 ppm.

MS-LC: MH+=415.0; r.t.=8.3 min.

### Example 75

### Mixture of 2-(4-bromophenyl)-5-{[2-(dimethylamino)ethyl]amino}-1,3-benzoxazole-4,7-dione and 2-(4-bromophenyl)-6-{[2-(dimethylamino)ethyl]amino}-1,3-benzoxazole-4,7-dione

The experimental protocol used is identical to that described for Example 74, N,N-dimethylethylenediamine replacing N-(2-aminoethyl)-pyrrolidine. Melting point: 184° C.

The two components of the mixture can be characterized by the NMR shifts (400 MHz) of the single proton of the benzoxazoledione ring which are 5.38 and 5.40 ppm.

MS-LC: MH+=390.1; r.t.=8.2 min.

### Example 76

### Mixture of 2-(4-fluorophenyl)-5-[(2-pyrrolidin-1-ylethyl)amino]-1,3-benzoxazole-4,7-dione and 2-(4-fluorophenyl)-6-[(2-pyrrolidin-1-lethyl)amino]-1,3-benzoxazole-4,7-dione

### 76.1) 2-(4-fluorophenyl)-6,7-dihydro-1,3-benzoxazol-4(5H)-one

The experimental protocol used is identical to that described for Stage 66.2, 4-fluorobenzonitrile replacing 2-fluorobenzonitrile. A yellow solid is obtained.

MS-LC: MH+=232.1; r.t.=9.4 min.

### 76.2) 5-bromo-2-(4-fluorophenyl)-6,7-dihydro-1,3-benzoxazol-4(5H)-one

Pyridinium tribromide (996 mg; 3.11 mmol) is added in three equal portions separated by intervals of 2-3 minutes to a solution of intermediate 76.1 (600 mg; 2.59 mmol) in glacial acetic acid (25 ml) taken to 50° C. The reaction mixture is maintained at 50° C. for 4 hours (TLC on SiO₂: AcOEt/heptane:1/1). The volatile constituents are evaporated under reduced pressure, then the residue is taken up in water and extracted with dichloromethane. The reaction medium is then filtered on Celite, the filtrate is evaporated under reduced pressure and the residue is purified on a column (SiO₂: AcOEt/heptane:1/1) in order to produce a light yellow powder. The organic phases are combined and washed with a 10% bicarbonate solution then with a saturated solution of sodium chloride. After drying over sodium sulphate and elimination of the volatile constituents under reduced pressure, the residue is purified by chromatography on a column (SiO₂: AcOEt/heptane:1/1) in order to produce a beige powder.

MS-LC: MH+=312.0; r.t.=10.3 min.

### 76.3) Mixture of 2-(4-fluorophenyl)-5-{[2-(dimethylamino)ethyl]amino}-1,3-benzoxazole-4,7-dione and 2-(4-fluorophenyl)-6-{[2-(dimethylamino)ethyl]amino}-1,3-benzoxazole-4,7-dione

This compound is obtained from intermediate 76.2 according to the operating methods described for Stages 66.4, 66.5 and 66.6. Melting point: 162° C.

The two components of the mixture can be characterized by the NMR shifts (400 MHz) of the single proton of the benzoxazoledione ring which are 5.37 and 5.39 ppm.

MS-LC: MH+=356.1; r.t.=8.0 min.

### Example 77

### Mixture of 5-{[2-(dimethylamino)ethyl]amino}-2-(4-fluorophenyl)-1,3-benzoxazole-4,7-dione and 6-{[2-(dimethylamino)ethyl]amino}-2-(4-fluorophenyl)-1,3-benzoxazole-4,7-dione

The experimental protocol used is identical to that described for Example 76, N-(2-aminoethyl)-pyrrolidine replacing N,N-dimethylethylenediamine. Melting point: 170° C.

The two components of the mixture can be characterized by the NMR shifts (400 MHz) of the single proton of the benzoxazoledione ring which are 5.38 and 5.39 ppm.

MS-LC: MH+=330.1; r.t.=7.8 min.

### Example 78

### Mixture of 5-[(1-benzylpyrrolidin-3-yl)amino]-2-(4-fluorophenyl)-1,3-benzoxazole-4,7-dione and 6-[(1-benzylpyrrolidin-3-yl)amino]-2-(4-fluorophenyl)-1,3-benzoxazole-4,7-dione

The experimental protocol used is identical to that described for Example 76, (1-benzylpyrrolidin-3-yl)-amine replacing N,N-dimethylethylenediamine. Melting point: 180° C.

The two components of the mixture can be characterized by the NMR shifts (400 MHz) of the single proton of the benzoxazoledione ring which are 5.37 and 5.39 ppm.

MS-LC: MH+=418.1; r.t.=8.5 min.

### Example 79

### Mixture of 5-{[3-(dimethylamino)propyl]amino}-2-(4-fluorophenyl)-1,3-benzoxazole-4,7-dione and 6-{[3-(dimethylamino)propyl]amino}-2-(4-fluorophenyl)-1,3-benzoxazole-4,7-dione

The experimental protocol used is identical to that described for Example 76,N,N-dimethylpropylenediamine replacing N,N-dimethylethylenediamine. Melting point: 149° C.

The two components of the mixture can be characterized by the NMR shifts (400 MHz) of the single proton of the benzoxazoledione ring which are 5.35 and 5.37 ppm.

MS-LC: MH+=344.2; r.t.=7.9 min.

### Example 80

### Mixture of 2-(3,5-difluorophenyl)-5-[(2-pyrrolidin-1-ylethyl)amino]-1,3-benzoxazole-4,7-dione and 2-(3,5-difluorophenyl)-6-[(2-pyrrolidin-1-ylethyl)amino]-1,3-benzoxazole-4,7-dione

The experimental protocol used is identical to that described for Example 68, 3,5-difluorobenzonitrile replacing 2-bromobenzonitrile and N-(2-aminoethyl)-pyrrolidine replacing N,N-dimethylethylenediamine. Melting point: 158° C.

The two components of the mixture can be characterized by the NMR shifts (400 MHz) of the single proton of the benzoxazoledione ring which are 5.41 and 5.43 ppm.

MS-LC: MH+=374.0; r.t.=8.0 min.

### Example 81

### Mixture of 2-(3,5-difluorophenyl)-5-{[2-(dimethylamino)ethyl]amino}-1,3-benzoxazole-4,7-dione and 2-(3,5-difluorophenyl)-6-{[2-(dimethylamino)ethyl]amino}-1,3-benzoxazole-4,7-dione

The experimental protocol used is identical to that described for Example 68, 3,5-difluorobenzonitrile replacing 2-bromobenzonitrile. Melting point: 175° C.

The two components of the mixture can be characterized by the NMR shifts (400 MHz) of the single proton of the benzoxazoledione ring which are 5.33 and 5.41 ppm.

MS-LC: MH+=348.0; r.t.=7.9 min.

### Example 82

### Mixture of 2-(2,5-difluorophenyl)-5-[(2-pyrrolidin-1-ylethyl)amino]-1,3-benzoxazole-4,7-dione and 2-(2,5-difluorophenyl)-6-[(2-pyrrolidin-1-ylethyl)amino]-1,3-benzoxazole-4,7-dione

The experimental protocol used is identical to that described for Example 68, 2,5-difluorobenzonitrile replacing 2-bromobenzonitrile and N-(2-aminoethyl)-pyrrolidine replacing N,N-dimethylethylenediamine.

Melting point: 163° C.

The two components of the mixture can be characterized by the NMR shifts (400 MHz) of the single proton of the benzoxazoledione ring which are 5.40 and 5.42 ppm.

MS-LC: MH+=374.0; r.t.=7.9 min.

### Example 83

### Mixture of 2-(2,5-difluorophenyl)-5-{[2-(dimethylamino)ethyl]amino}-1,3-benzoxazole-4,7-dione and 2-(2,5-difluorophenyl)-6-{[2-(dimethylamino)ethyl]amino}-1,3-benzoxazole-4,7-dione

The experimental protocol used is identical to that described for Example 68, 2,5-difluorobenzonitrile replacing 2-bromobenzonitrile.

The two components of the mixture can be characterized by the NMR shifts (400 MHz) of the single proton of the benzoxazoledione ring which are 5.41 and 5.43 ppm.

MS-LC: MH+=348.0; r.t.=7.7 min.

### Example 84

### Mixture of 2-(2,3-difluorophenyl)-5-{[2-(dimethylamino)ethyl]amino}-1,3-benzoxazole-4,7-dione and 2-(2,3-difluorophenyl)-6-{[2-(dimethylamino)ethyl]amino}-1,3-benzoxazole-4,7-dione

The experimental protocol used is identical to that described for Example 68, 2,3-difluorobenzonitrile replacing 2-bromobenzonitrile. Melting point: 167° C.

The two components of the mixture can be characterized by the NMR shifts (400 MHz) of the single proton of the benzoxazoledione ring which are 5.41 and 5.43 ppm.

MS-LC: MH+=348.1; r.t.=7.8 min.

### Example 85

### Mixture of 2-(2,3-difluorophenyl)-5-[(2-pyrrolidin-1-ylethyl)amino]-1,3-benzoxazole-4,7-dione and 2-(2,3-difluorophenyl)-6-[(2-pyrrolidin-1-ylethyl)amino]-1,3-benzoxazole-4,7-dione

The experimental protocol used is identical to that described for Example 68, 2,3-difluorobenzonitrile replacing 2-bromobenzonitrile and N-(2-aminoethyl)-pyrrolidine replacing N,N-dimethylethylenediamine. Melting point: 150° C.

The two components of the mixture can be characterized by the NMR shifts (400 MHz) of the single proton of the benzoxazoledione ring which are 5.40 and 5.42 ppm.

MS-LC: MH+=374.1; r.t.=7.9 min.

### Example 86

### Mixture of 2-(2,3-difluorophenyl)-5-{[3-(dimethylamino)propyl]amino}-1,3-benzoxazole-4,7-dione and 2-(2,3-difluorophenyl)-6-{[3-(dimethylamino)propyl]amino}-1,3-benzoxazole-4,7-dione

The experimental protocol used is identical to that described for Example 68, 2,3-difluorobenzonitrile replacing 2-bromobenzonitrile, and N,N dimethylpropylenediamine replacing N,N-dimethylethylenediamine. Melting point: 169° C.

The two components of the mixture can be characterized by the NMR shifts (400 MHz) of the single proton of the benzoxazoledione ring which are 5.38 and 5.41 ppm.

MS-LC: MH+=362.1; r.t.=7.8 min.

### Example 87

### Mixture of 5-[(2-pyrrolidin-1-ylethyl)amino]-2-(3,4,5-trifluorophenyl)-1,3-benzoxazole-4,7-dione and 6-[(2-pyrrolidin-1-ylethyl)amino]-2-(3,4,5-trifluorophenyl)-1,3-benzoxazole-4,7-dione

The experimental protocol used is identical to that described for Example 68, 3,4,5-trifluorobenzonitrile replacing 2-bromobenzonitrile, and N-(2-aminoethyl)-pyrrolidine replacing N,N-dimethylethylenediamine.

The two components of the mixture can be characterized by the NMR shifts (400 MHz) of the single proton of the benzoxazoledione ring which are 5.39 and 5.41 ppm.

MS-LC: MH+=392.0; r.t.=8.2 min.

### Example 88

### Mixture of 5-{[2-(dimethylamino)ethyl]amino}-2-(3,4,5-trifluorophenyl)-1,3-benzoxazole-4,7-dione and 6-{[2-(dimethylamino)ethyl]amino}-2-(3,4,5-trifluorophenyl)-1,3-benzoxazole-4,7-dione

The experimental protocol used is identical to that described for Example 68, 3,4,5-trifluorobenzonitrile replacing 2-bromobenzonitrile.

The two components of the mixture can be characterized by the NMR shifts (400 MHz) of the single proton of the benzoxazoledione ring which are 5.40 and 5.42 ppm.

MS-LC: MH+=366.1; r.t.=8.1 min.

### Example 89

### Mixture of 5-[(2-pyrrolidin-1-ylethyl)amino]-2-(2,3,4,5-tetrafluorophenyl)-1,3-benzoxazole-4,7-dione and 6-[(2-pyrrolidin-1-ylethyl)amino]-2-(2,3,4,5-tetrafluorophenyl)-1,3-benzoxazole-4,7-dione

The experimental protocol used is identical to that described for Example 68, 2,3,4,5-tetrafluorobenzonitrile replacing 2-bromobenzonitrile and N-(2-aminoethyl)-pyrrolidine replacing N,N-dimethylethylenediamine.

The two components of the mixture can be characterized by the NMR shifts (400 MHz)

of the single proton of the benzoxazoledione ring which are 5.42 and 5.44 ppm.

MS-LC: MH+=410.0; r.t.=8.2 min.

### Example 90

### Mixture of 5-{[2-(dimethylamino)ethyl]amino}-2-(2,3,4,5-tetrafluorophenyl)-1,3-benzoxazole-4,7-dione and 6-{[2-(dimethylamino)ethyl]amino}-2-(2,3,4,5-tetrafluorophenyl)-1,3-benzoxazole-4,7-dione

The experimental protocol used is identical to that described for Example 68, 2,3,4,5-tetrafluorobenzonitrile replacing 2-bromobenzonitrile. Melting point: 160° C.

The two components of the mixture can be characterized by the NMR shifts (400 MHz) of the single proton of the benzoxazoledione ring which are 5.42 and 5.45 ppm.

MS-LC: MH+=384.0; r.t.=8.1 min.

### Example 91

### Mixture of 5-{[2-(dimethylamino)ethyl]amino}-2-[2-fluoro-6-(trifluoromethyl)phenyl]-1,3-benzoxazole-4,7-dione and 6-{[2-(dimethylamino)ethyl]amino}-2-[2-fluoro-6-(trifluoromethyl)phenyl]-1,3-benzoxazole-4,7-dione

The experimental protocol used is identical to that described for Example 68, 2-fluoro-6-(trifluoromethyl)-benzonitrile replacing 2-bromobenzonitrile.

The two components of the mixture can be characterized by the NMR shifts (400 MHz)

of the single proton of the benzoxazoledione ring which are 5.44 and 5.46 ppm.

MS-LC: MH+=398.0; r.t.=8.0 min.

### Example 92

### Mixture of 2-[2-fluoro-6-(trifluoromethyl)phenyl]-5-[(2-pyrrolidin-1-ylethyl)amino]-1,3-benzoxazole-4,7-dione and 2-[2-fluoro-6-(trifluoromethyl)phenyl]-6-[(2-pyrrolidin-1-ylethyl)amino]-1,3-benzoxazole-4,7-dione

The experimental protocol used is identical to that described for Example 68, 2-fluoro-6-(trifluoromethyl)-benzonitrile replacing 2-bromobenzonitrile and N-(2-aminoethyl)-pyrrolidine replacing N,N-dimethylethylenediamine. Melting point: 166° C.

The two components of the mixture can be characterized by the NMR shifts (400 MHz) of the single proton of the benzoxazoledione ring which are 5.43 and 5.45 ppm.

MS-LC: MH+=424.1; r.t.=8.1 min.

### Example 93

### Mixture of 5-{[3-(dimethylamino)propyl]amino}-2-[2-fluoro-6-(trifluoromethyl)phenyl]-1,3-benzoxazole-4,7-dione and 6-{[3-(dimethylamino)propyl]amino}-2-[2-fluoro-6-(trifluoromethyl)phenyl]-1,3-benzoxazole-4,7-dione

The experimental protocol used is identical to that described for Example 68, 2-fluoro-6-(trifluoromethyl)-benzonitrile replacing 2-bromobenzonitrile and N,N-dimethylpropylenediamine replacing N,N-dimethylethylenediamine. Melting point: 128° C.

The two components of the mixture can be characterized by the NMR shifts (400 MHz) of the single proton of the benzoxazoledione ring which are 5.42 and 5.43 ppm.

MS-LC: MH+=412.0; r.t.=8.0 min.

### Example 94

### Mixture of 2-[2-chloro-5-(trifluoromethyl)phenyl]-5-{[2-(dimethylamino)ethyl]amino}-1,3-benzoxazole-4,7-dione and 2-[2-chloro-5-(trifluoromethyl)phenyl]-6-{[2-(dimethylamino)ethyl]amino}-1,3-benzoxazole-4,7-dione

The experimental protocol used is identical to that described for Example 68, 2-chloro-5-(trifluoromethyl)-benzonitrile replacing 2-bromobenzonitrile. Melting point: 182° C.

The two components of the mixture can be characterized by the NMR shifts (400 MHz) of the single proton of the benzoxazoledione ring which are 5.43 and 5.46 ppm.

MS-LC: MH+=414.0; r.t.=8.3 min.

### Example 95

### Mixture of 2-[2-chloro-5-(trifluoromethyl)phenyl]-5-[(2-pyrrolidin-1-ylethyl)amino]-1,3-benzoxazole-4,7-dione and 2-[2-chloro-5-(trifluoromethyl)phenyl]-6-[(2-pyrrolidin-1-ylethyl)amino]-1,3-benzoxazole-4,7-dione

The experimental protocol used is identical to that described for Example 68, 2-chloro-5-(trifluoromethyl)-benzonitrile replacing 2-bromobenzonitrile and N-(2-aminoethyl)-pyrrolidine replacing N,N-dimethylethylenediamine. Melting point: 152° C.

The two components of the mixture can be characterized by the NMR shifts (400 MHz) of the single proton of the benzoxazoledione ring which are 5.43 and 5.45 ppm.

MS-LC: MH+=440.0; r.t.=8.5 min.

### Example 96

### Mixture of 2-[2-chloro-5-(trifluoromethyl)phenyl]-5-{[3-(dimethylamino)propyl]amino}-1,3-benzoxazole-4,7-dione and 2-[2-chloro-5-(trifluoromethyl)phenyl]-6-{[3-(dimethylamino)propyl]amino}-1,3-benzoxazole-4,7-dione

The experimental protocol used is identical to that described for Example 68, 2-chloro-5-(trifluoromethyl)-benzonitrile replacing 2-bromobenzonitrile and N,N-dimethylpropylenediamine replacing N,N-dimethylethylenediamine. Melting point: 121° C.

The two components of the mixture can be characterized by the NMR shifts (400 MHz) of the single proton of the benzoxazoledione ring which are 5.41 and 5.43 ppm.

MS-LC: MH+=428.0; r.t.=8.4 min.

### Example 97

### Mixture of 2-[2-chloro-6-fluorophenyl]-5-{[3-(dimethylamino)propyl]amino}-1,3-benzoxazole-4,7-dione and 2-[2-chloro-6-fluorophenyl]-6-{[3-(dimethylamino)propyl]amino}-1,3-benzoxazole-4,7-dione

The experimental protocol used is identical to that described for Example 68, 2-chloro-6-fluorobenzonitrile replacing 2-bromobenzonitrile.

The two components of the mixture can be characterized by the NMR shifts (400 MHz)

of the single proton of the benzoxazoledione ring which are 5.43 and 5.45 ppm.

MS-LC: MH+=364.1; r.t.=7.8 min.

### Example 98

### Mixture of 2-[2-chloro-6-fluorophenyl]-5-[(2-pyrrolidin-1-ylethyl)amino]-1,3-benzoxazole-4,7-dione and 2-(2-chloro-6-fluorophenyl]-6-[(2-pyrrolidin-1-ylethyl)amino]-1,3-benzoxazole-4,7-dione

The experimental protocol used is identical to that described for Example 68, 2-chloro-6-fluorobenzonitrile replacing 2-bromobenzonitrile and N-(2-aminoethyl)-pyrrolidine replacing N,N-dimethylethylenediamine. Melting point: 124° C.

The two components of the mixture can be characterized by the NMR shifts (400 MHz) of the single proton of the benzoxazoledione ring which are 5.42 and 5.44 ppm.

MS-LC: MH+=390.1; r.t.=7.9 min.

### Example 99

### Mixture of 2-[3,4-dimethoxyphenyl]-5-{[2-(dimethylamino)ethyl]amino}-1,3-benzoxazole-4,7-dione and 2-[3,4-dimethoxyphenyl]-6-{[2-(dimethylamino)ethyl]amino}-1,3-benzoxazole-4,7-dione

### 99.1) 2-(3,4-dimethoxyphenyl)-6,7-dihydro-1,3-benzoxazol-4(5H)-one

The experimental protocol used is identical to that described for Stage 66.2, 3,4-dimethoxybenzonitrile replacing 2-fluorobenzonitrile. A yellow solid is obtained.

MS-LC: MH+=274.0; r.t.=8.9 min.

### 99.2) 5-iodo-2-(3,4-dimethoxyphenyl)-6,7-dihydro-1,3-benzoxazol-4(5H)-one

A solution of intermediate 99.1 (500 mg, 1.83 mmol) in acetic acid (30 ml) is treated for 96 hours at ambient temperature with poly[styrene-co-(4-vinylpyridinium dichloroiodate(1-))] (2.6 g; 8.25 mEq; prepared according to B S\check{k}et et al., Bull. Chem. Soc. Jpn (1989), 62, 3406-3408) (TLC verification on SiO₂: 2% MeOH/CH₂Cl₂). The polymer is removed by filtration and the volatile constituents are evaporated under reduced pressure. The residue is purified on a column (SiO₂: 1% MeOH/CH₂Cl₂) in order to produce a yellow oil.

MS-LC: MH+=399.9; r.t.=9.8 min.

### 99.3) Mixture of 2-(3,4-dimethoxyphenyl)-5-{[2-(dimethylamino)ethyl]amino}-1,3-benzoxazole-4,7-dione and 2-(3,4-dimethoxyphenyl)-6-{[2-(dimethylamino)ethyl]amino}-1,3-benzoxazole-4,7-dione

This compound is obtained from intermediate 99.2 according to the operating methods described for Stages 66.4, 66.5 and 66.6. Melting point: 181° C.

The two components of the mixture can be characterized by the NMR shifts (400 MHz) of the single proton of the benzoxazoledione ring which are 5.35 and 5.36 ppm.

MS-LC: MH+=372.1; r.t.=7.6 min.

### Example 100

### Mixture of 2-[2-bromo-3-pyridyl]-5-{[2-(dimethylamino)ethyl]amino}-1,3-benzoxazole-4,7-dione and 2-[2-bromo 3-pyridyl]-6-{[2-(dimethylamino)ethyl]amino}-1,3-benzoxazole-4,7-dione

The experimental protocol used is identical to that described for Example 68, 2-bromonicotinonitrile replacing 2-bromobenzonitrile. Melting point: 133° C.

The two components of the mixture can be characterized by the NMR shifts (400 MHz) of the single proton of the benzoxazoledione ring which are 5.43 and 5.45 ppm.

MS-LC: MH+=391.0; r.t.=7.4 min.

### Example 101

### Mixture of 2-cyclohexyl-5-[(2-pyrrolidin-1-ylethyl)amino]-1,3-benzothiazole-4,7-dione and 2-cyclohexyl-6-[(2-pyrrolidin-1-ylethyl)amino]-1,3-benzothiazole-4,7-dione

### 101.1) N-(2,5-dimethoxyphenyl)cyclohexanecarboxamide

1 ml (7.62 mmol, 1.1 eq.) of cyclohexanoic acid chloride is added to a solution of 1.05 g (6.89 mmol) of 2,5-dimethoxyaniline in 10 ml of a mixture toluene/methanol (1/1). The reaction mixture is maintained under stirring at 70° C. for 1.5 hour, and, after returning to ambient temperature, is poured into 50 ml of water. The expected product is extracted twice with 50 ml of toluene, then washed twice with 50 ml of water. The organic phases are combined, dried over magnesium sulphate and the solvent evaporated off under reduced pressure. 1.46 g (yield=67%) of N-(2,5-dimethoxyphenyl)cyclohexanecarboxamide is obtained and used without other purification in the following stage.

NMR ¹H (DMSO d6, 400 MHz, δ): 8.84 (s, 1H, NH); 7.72-7.71 (m, 1H, H arom.); 6.93-6.91 (d, 1H, H arom.); 6.60-6.57 (m, 1H, H arom.); 3.76 (s, 3H, CH₃); 3.66 (s, 3H, CH₃); 1.78-1.70 (m, 6H, CH₂, CH); 1.38-1.24 (m, 5H, CH₂).

MS-LC: MH+=264.14; r.t.=10.76 min.

### 101.2) N-(2,5-dimethoxyphenyl)cyclohexanecarbothioamide

1.46 g (5.54 mmol) of N-(2,5-dimethoxyphenyl)cyclohexanecarboxamide is placed in solution in 40 ml of anhydrous toluene. The solution is taken to 100° C., and 3.34 g (8.26 mmol; 1.5 eq.) of Lawesson's reagent are added to the reaction medium which is then maintained under stirring at 100° C. for 4 hours. After returning to ambient temperature, the solution is poured into 50 ml of iced water and extracted using toluene. The organic phases are dried over magnesium sulphate and the solvent is evaporated off. The N-(2,5-dimethoxyphenyl)cyclohexanecarbothioamide is then purified by chromatography on a silica column (eluent: dichloromethane/heptane:1/1 then 3/2). 1.26 g (yield=81%) of product is obtained in the form of yellow oil.

NMR ¹H (DMSO d6, 400 MHz, δ): 10.76 (s, 1H, NH); 7.28-7.27 (m, 1H, H arom.); 7.02-6.99 (d, 1H, H arom.); 6.82-6.80 (m, 1H, H arom.); 3.73 (s, 3H, CH₃); 3.68 (s, 3H, CH₃); 1.77-1.75 (m, 4H, CH₂); 1.67-1.58 (m, 3H, CH₂, CH); 1.31-1.15 (m, 4H, 2CH₂).

MS-LC: MH+=280.12; r.t.=11.38 min.

### 101.3) 2-cyclohexyl-4,7-dimethoxy-1,3-benzothiazole

1.26 g (4.50 mmol) of N-(2,5-dimethoxyphenyl)cyclohexanecarbothioamide is dissolved in 100 ml of a 1.5 M sodium hydroxide solution (100 ml) and the reaction medium is cooled down to 0° C. before adding 25 ml of a freshly prepared 20% aqueous solution of potassium ferricyanide (5.05 g of K₃[Fe(CN)₆]; 3.4 eq.). The reaction mixture is maintained under stirring at ambient temperature for 24 hours, then 1.1 g (yield=88%) of the expected benzothiazole derivative is obtained by filtration, washing with cold water and drying under reduced pressure in the presence of P₂O₅.

NMR ¹H (DMSO d6, 400 MHz, δ): 6.95-6.85 (dd, 2H, H arom.); 3.88 (s, 6H, 2CH₃); (m, 1H, CH); 2.10-2.07 (m, 2H, CH₂); 1.81-1.77 (m, 2H, CH₂); 1.70-1.67 (m, 1H, CH); 1.57-1.51 (m, 2H, CH₂); 1.42-1.39 (m, 2H, CH₂); 1.26-1.28 (m, 1H, CH).

MS-LC: MH+=278.09; r.t.=11.91 min.

### 101.4) 2-cyclohexyl-1,3-benzothiazole-4,7-dione

1 g (3.61 mmol) of 2-cyclohexyl-4,7-dimethoxy-1,3-benzothiazole is put into suspension in an acetonitrile/water mixture (3/1) at 0° C. then 4.36 g (7.96 mmol; 2.2 eq.) of cerium (IV) and ammonium nitrate are added to the suspension. The reaction mixture is maintained for 1.5 hours under stirring at ambient temperature, then 0.78 g (yield=88%) of 2-cyclohexyl-1,3-benzothiazole-4,7-dione is obtained after filtration, washing with cold water and drying under reduced pressure.

NMR ¹H (DMSO d6, 400 MHz, δ): 6.90 (s, 2H); 3.15-3.10 (m, 1H, CH); 2.10-2.07 (m, 2H, CH₂); 1.81-1.77 (m, 2H, CH₂); 1.65-1.70 (m, 1H, CH); 1.55-1.39 (m, 5H, CH, CH₂).

MS-LC: MH+=248.12; r.t.=10.82 min.

### 101.5) N-(2,5-dimethoxyphenyl)cyclohexanecarboxamide

The experimental protocol used is identical to that described for Stage 15.4 of Example 15, intermediate 101.4 replacing intermediate 15.3 and N,N-dimethylethylene diamine replacing aniline. A mixture of 80% and 9% of 2-cyclohexyl-5-{[2-(dimethylamino)ethyl]amino}-1,3-benzothiazole-4,7-dione and of 2-cyclohexyl-6-{[2-(dimethylamino)ethyl]amino}-1,3-benzothiazole-4,7-dione is obtained.

NMR ¹H (DMSO d6, 400 MHz, δ): 7.20 (t, 1H, NH); 5.49 and 5.43 (2s, H); 3.24-3.21 (m, 2H, CH₂); 3.09-3.12 (m, 3H, CH, CH₂); 2.19 (s, 6H, 2CH₃); 2.09-2.06 (m, 2H, CH₂); 1.80-1.77 (m, 3H, CH, CH₂); 1.53-1.49 (m, 4H, 2CH₂); 1.41-1.38 (m, 1H, CH).

MS-LC: MH+=334.17; r.t.=7.99 and 8.06 min.

The compounds of Examples 102 to 113 are obtained in a similar manner to that described for Example 101.

### Example 102

### Mixture of 2-cyclohexyl-5-[(2-pyrrolidin-1-ylethyl)amino]-1,3-benzothiazole-4,7-dione and 2-cyclohexyl-6-[(2-pyrrolidin-1-ylethyl)amino]-1,3-benzothiazole-4,7-dione

MS-LC: MH+=360.16; r.t.=8.14 and 8.19 min.

### Example 103

### Mixture of 5-[(2-pyrrolidin-1-ylethyl)amino]-2-thien-2-yl-1,3-benzothiazole-4,7-dione and 6-[(2-pyrrolidin-1-ylethyl)amino]-2-thien-2-yl-1,3-benzothiazole-4,7-dione

MS-LC: MH+=360.01; r.t.=7.78 and 7.86 min.

### Example 104

### Mixture of 2-(2,5-dichlorothien-3-yl)-5-{(2-(dimethylamino)ethyl]amino}-1,3-benzothiazole-4,7-dione and 2-(2,5-dichlorothien-3-yl)-6-{[2-(dimethylamino)ethyl]amino}-1,3-benzothiazole-4,7-dione

MS-LC: MH+=401.86; r.t.=8.44 and 8.59 min.

### Example 105

### Mixture of 2-(2,5-dichlorothien-3-yl)-5-[(2-pyrrolidin-1-ylethyl)amino]-1,3-benzothiazole-4,7-dione and 2-(2,5-dichlorothien-3-yl)-6-[(2-pyrrolidin-1-ylethyl)amino]-1,3-benzothiazole-4,7-dione

MS-LC: MH+=427.87; r.t.=8.63 and 8.80 min.

### Example 106

### Mixture of 2-(2-furyl)-5-[(2-pyrrolidin-1-ylethyl)amino]-1,3-benzothiazole-4,7-dione and 2-(2-furyl)-6-[(2-pyrrolidin-1-ylethyl)amino]-1,3-benzothiazole-4,7-dione

MS-LC: MH+=344.04; r.t.=7.57 and 7.64 min.

### Example 107

### Mixture of 5-{[2-(dimethylamino)ethyl]amino}-2-(2-methoxypheDyl)-1,3-benzothiazole-4,7-dione and 6-{[2-(dimethylamino)ethyl]amino}-2-(2-methoxyphenyl)-1,3-benzothiazole-4,7-dione

MS-LC: MH+=358.18; r.t.=7.88 and 7.97 min.

### Example 108

### Mixture of 5-{[2-(dimethylamino)ethyl]amino}-2-(2-fluorophenyl)-1,3-benzothiazole-4,7-dione and 6-{(2-{dimethylamino)ethyl]amino}-2-(2-fluorophenyl)-1,3-benzothiazole-4,7-dione

MS-LC: MH+=346.14; r.t.=7.85 and 7.94 min.

### Example 109

### Mixture of 2-(2-fluorophenyl)-5-[(2-pyrrolidin-1-ylethyl)amino]-1,3-benzothiazole-4,7-dione and 2-(2-fluorophenyl)-6-[(2-pyrrolidin-1-ylethyl)amino]-1,3-benzothiazole-4,7-dione

MS-LC: MH+=372.14; r.t.=7.97 and 8.06 min.

### Example 110

### Mixture of 2-(4-fluorophenyl)-5-[(2-pyrrolidin-1-ylethyl)amino]-1,3-benzothiazole-4,7-dione and 2-(4-fluorophenyl)-6-[(2-pyrrolidin-1-ylethyl)amino]-1,3-benzothiazole-4,7-dione

MS-LC: MH+=372.05; r.t.=7.98 and 8.07 min.

### Example 111

### Mixture of 5-{[2-(dimethylamino)ethyl]amino}-2-(4-fluorophenyl)-1,3-benzothiazole-4,7-dione and 6-{[2-(dimethylamino)ethyl]amino}-2-(4-fluorophenyl)-1,3-benzothiazole-4,7-dione

MS-LC: MH+=346.05; r.t.=7.87 and 7.95 min.

### Example 112

### Mixture of 2-(2,6-difluorophenyl)-5-[(2-pyrrolidin-1-ylethyl)amino]-1,3-benzothiazole-4,7-dione and 2-(2,6-difluorophenyl)-6-[(2-pyrrolidin-1-ylethyl)amino]-1,3-benzothiazole-4,7-dione

MS-LC: MH+=390.04; r.t.=7.89 and 7.95 min.

### Example 113

### Mixture of 2-(2,6˜difluorophenyl)-5-{[2-(dimethylamino)ethyl]amino}-1,3-benzothiazole-4,7-dione and 2-(2,6-difluorophenyl)-6-{[2-(dimethylamino)ethyl]amino}-1,3-benzothiazole-4,7-dione

MS-LC: MH+=364.05; r.t.=7.78 and 7.83 min.

### Example 114

### 5-[[2-(dimethylamino)ethyl](ethyl)amino]-2-methyl-1,3-benzothiazole-4,7-dione

This compound is obtained in a similar manner to that described for Example 1, N,N,N′-trimethylethylenediamine replacing 4-(2-aminoethyl)morpholine.

NMR ¹H (DMSO d6, 400 MHz, δ): 5.53 (s, 1H, CH); 3.73-3.70 (t, 2H, CH₂); 2.93 (s, 3H, CH₃); 2.74 (s, 3H, CH₃); 2.32-2.30 (t, 2H, CH₂); 1.92 (s, 6H, 2CH₃).

MS-LC: MH+=280.11; r.t.=7.03 min.

### Example 115

### 5-[[2-(dimethylamino)ethyl](methyl)amino]-2-methyl-1,3-benzothiazole-4,7-dione

This compound is obtained in a similar manner to that described for Example 1, N,N-dimethyl-N′-ethylenediamine replacing 4-(2-aminoethyl)morpholine.

MS-LC: MH+=294.07; r.t.=7.20 min.

### Example 116

### Mixture of 2-[2,6-dichloro-5-fluoro-3-pyridyl]-5-{(2-(dimethylamino)ethyl]amino}-1,3-benzoxazole-4,7-dione and 2-[2,6-dichloro-5-fluoro-3-pyridyl]-6-{[2-(dimethylamino)ethyl]amino}-1,3-benzoxazole-4,7-dione

The experimental protocol used is identical to that described for Example 68, 2,6-dichloro-5-fluoronicotinonitrile replacing 2-bromobenzonitrile.

MS-LC: MH+=399.1; r.t.=8.1 min.

### Example 117

### Mixture of 2-[2,6-dichloro-5-fluoro-3-pyridyl]-5-[(2-pyrrolidin-1-ylethyl)amino]-1,3-benzoxazole-4,7-dione and 2-[2,6-dichloro-5-fluoro-3-pyridyl]-6-[(2-pyrrolidin-1-ylethyl)amino]-1,3-benzoxazole-4,7-dione

The experimental protocol used is identical to that described for Example 68, 2,6-dichloro-5-fluoronicotinonitrile replacing 2-bromobenzonitrile and N-(2-aminoethyl)-pyrrolidine replacing N,N-dimethylethylenediamine.

MS-LC: MH+=399.1; r.t.=8.1 min.

### Example 118

### Mixture of 2-(2,4-difluorophenyl)-5-{[2-(dimethylamino)ethyl]amino}-1,3-benzoxazole-4,7-dione and 2-(2,4-difluorophenyl)-6-{[2-(dimethylamino)ethyl]amino}-1,3-benzoxazole-4,7-dione

The experimental protocol used is identical to that described for Example 68, 2,4-difluorobenzonitrile replacing 2-bromobenzonitrile.

MS-LC: MH+=348.1; r.t.=7.8 min.

### Example 119

### Mixture of 5-{[2-(dimethylamino)ethyl]amino}-2-(2,3,4-trifluorophenyl)-1,3-benzoxazole-4,7-dione and 6-{[2-(dimethylamino)ethyl]amino}-2-(2,3,4-trifluorophenyl)-1,3-benzoxazole-4,7-dione

The experimental protocol used is identical to that described for Example 68, 2,3,4-trifluorobenzonitrile replacing 2-bromobenzonitrile. Melting point: 156° C.

MS-LC: MH+=366.1; r.t.=8.0 min.

### Example 120

### Mixture of 5-[(2-pyrrolidin-1-ylethyl)amino]-2-(2,3,4-trifluorophenyl)-1,3-benzoxazole-4,7-dione and 6-[(2-pyrrolidin-1-ylethyl)amino]-2-(2,3,4-trifluorophenyl)-1,3-benzoxazole-4,7-dione

The experimental protocol used is identical to that described for Example 68, 2,3,4-trifluorobenzonitrile replacing 2-bromobenzonitrile and N-(2-aminoethyl)-pyrrolidine replacing N,N-dimethylethylenediamine.

MS-LC: MH+=392.1; r.t.=8.1 min.

### Example 121

### Mixture of 2-(3-fluoro-4-methylphenyl)-5-{[2-(dimethylamino)ethyl]amino}-1,3-benzoxazole-4,7-dione and 2-(3-fluoro-4-methylphenyl)-6-{[2-(dimethylamino)ethyl]amino}-1,3-benzoxazole-4,7-dione

The experimental protocol used is identical to that described for Example 68, 3-fluoro-4-methylbenzonitrile replacing 2-bromobenzonitrile. Melting point: 179° C.

MS-LC: MH+=344.1; r.t.=8.1 min.

### Example 122

### Mixture of 2-(3-fluoro-4-methylphenyl)-5-[(2-pyrrolidin1-ylethyl)amino]-1,3-benzoxazole-4,7-dione and 2-(3-fluoro-4-methylphenyl)-6-[(2-pyrrolidin-1-ylethyl)amino]-1,3-benzoxazole-4,7-dione

The experimental protocol used is identical to that described for Example 68, 3-fluoro-4-methylbenzonitrile replacing 2-bromobenzonitrile and N-(2-aminoethyl)-pyrrolidine replacing N,N-dimethylethylenediamine.

MS-LC: MH+=370.1; r.t.=8.2 min.

The compounds of Examples 123 to 127 are obtained in a similar manner to that described for Example 101.

### Example 123

### Mixture of 2-(4-clorophenyl)-5-{[2-(dimethylamino)ethyl]amino}-1,3-benzothiazole-4,7-dione and 2-(4-chlorophenyl)-6-{[2-(dimethylamino)ethyl]amino}-1,3-benzothiazole-4,7-dione

MS-LC: MH+=362.07; r.t.=8.11 and 8.20 min.

### Example 124

### Mixture of 2-(4-chlorophenyl)-5-[(2-pyrrolidin-1-ylethyl)amino]-1,3-benzothiazole-4,7-dione and 2-(4-chlorophenyl)-6-[(2-pyrrolidin-1-ylethyl)amino]-1,3-benzothiazole-4,7-dione

MS-LC: MH+=388.04; r.t.=8.23 and 8.34 min.

### Example 125

### Mixture of 5-{[2-(dimethylamino)ethyl]amino}-2-(2,3,4,5-tetrafluorophenyl)-1,3-benzothiazole-4,7-dione and 6-{[2-(dimethylamino)ethyl]amino}-2-(2,3,4,5-tetrafluorophenyl)-1,3-benzothiazole-4,7-dione

MS-LC: MH+=400.01; r.t.=8.23 and 8.32 min.

### Example 126

### Mixture of 5-{[2-(dimethylamino)ethyl]amino}-2-(3,4,5-trifluorophenyl)-1,3-benzothiazole-4,7-dione and 6-{[2-(dimethylamino)ethyl]amino}-2-(3,4,5-trifluorophenyl)-1,3-benzothiazole-4,7-dione

MS-LC: MH+=382.03; r.t.=8.10 and 8.19 min.

### Example 127

### Mixture of 5-[(2-pyrrolidin-1-ylethyl)amino]-2-(2,4,6-trifluorophenyl)-1,3-benzothiazole-4,7-dione and 6-[(2-pyrrolidin-1-ylethyl)amino]-2-(2,4,6-trifluorophenyl)-1,3-benzothiazole-4,7-dione

MS-LC: MH+=408.02; r.t.=7.97 and 8.05 min.

The compounds of Examples 128 to 131 are obtained in a similar manner to that described for Example 66.

### Example 128

### Mixture of 2-(1,3-benzodioxol-5-yl)-5-{[2-(dimethylamino)ethyl]amino}-1,3-benzoxazole-4,7-dione and 2-(1,3-benzodioxol-5-yl)-6-{[2-(dimethylamino)ethyl]amino}-1,3-benzoxazole-4,7-dione

The two components of the mixture can be characterized by the NMR shifts (400 MHz) of the single proton of the benzoxazoledione ring which are 5.35 and 5.37 ppm.

MS-LC: MH+=356.07; r.t.=7.72 min.

### Example 129

### Mixture of 5-{[2-(dimethylamino)ethyl]amino}-2-(4-ethylphenyl)-1,3-benzoxazole-4,7-dione and of 6-{(2-(dimethylamino)ethyl]amino}-2-(4-ethylphenyl)-1,3-benzoxazole-4,7-dione

The two components of the mixture can be characterized by the NMR shifts (400 MHz) of the single proton of the benzoxazoledione ring which are 5.36 and 5.38 ppm.

MS-LC: MH+=340.18; r.t.=8.24 min.

### Example 130

### Mixture of 2-(4-ethylphenyl)-5-[(2-pyrrolidin-1-ylethyl)amino]-1,3-benzoxazole-4,7-dione and 2-(4-ethylphenyl)-6-[(2-pyrrolidin-1-ylethyl)amino]-1,3-benzoxazole-4,7-dione

The two components of the mixture can be characterized by the NMR shifts (400 MHz) of the single proton of the benzoxazoledione ring which are 5.35 and 5.36 ppm.

MS-LC: MH+=366.15; r.t.=8.34 min.

### Example 131

### Mixture of 5-{[2-(dimethylamino)ethyl]amino}-2-(2-fluoro-6-methoxyphenyl)-1,3-benzoxazole-4,7-dione and 6-{[2-(dimethylamino)ethyl]amino}-2-(2-fluoro-6-methoxyphenyl)-1,3-benzoxazole-4,7-dione

The two components of the mixture can be characterized by the NMR shifts (400 MHz) of the single proton of the benzoxazoledione ring which are 5.39 and 5.40 ppm.

MS-LC: MH+=360.09; r.t.=7.67 min.

### Example 132

### 2-(2,6-difluorophenyl)-5-{[2-(dimethylamino)ethyl]amino}-1,3-benzothiazole-4,7-dione

### 132.1) N-(3,5-dimethoxyphenyl)-2,6-difluorobenzamide

5.5 ml (39.2 mmol; 1.2 equivalent) of triethylamine and 4.5 ml (35.9 mmol; 1.1 equivalent) of 2,6-difluorobenzoyl chloride are added to 5 g (32.6 mmol) of 3,5-dimethoxyaniline in solution in 100 ml of anhydrous toluene. The reaction medium is maintained under stirring at 70° C. for 1 hour 30 minutes, then, after returning to ambient temperature, is washed with 3 times 50 ml of water. The resulting organic phase is dried over magnesium sulphate then the solvent is evaporated off under reduced pressure. The expected product is obtained in the form of a white powder (8.75 g; yield 97%) used in the following stage without other purification.

MS-LC: MH+=294.11; r.t.=9.93 min.

### 132.2) N-(3,5-dimethoxyphenyl)-2,6-difluorobenzenecarbothioamide

20.3 g (50 mmol; 1.5 equivalents) of Lawesson's reagent is added to 9.8 g (33.4 mmol) of N-(3,5-dimethoxyphenyl)-2,6-difluorobenzamide in solution in 150 ml of anhydrous toluene. The reaction medium is maintained under stirring at 120° C. for 8 hours, then, after returning to ambient temperature, is washed with 3 times 75 ml of water. The resulting organic phase is dried over magnesium sulphate then the solvent is evaporated off under reduced pressure. The residue is purified by chromatography on a silica column (eluent: dichloromethane/methanol 98/2) and the expected product is obtained in the form of a green oil (10 g; yield=96%).

MS-LC: MH+=310.06; r.t.=10.53 min.

### 132.3) 2-(2,6-difluorophenyl}-5,7-dimethoxy-1,3-benzothiazole

170 ml (103 mmol; 3 equivalents) of a freshly prepared 20% aqueous solution of potassium ferricyanide is added to 10.3 g (33.3 mmol) of N-(3,5-dimethoxyphenyl)-2,6-difluorobenzenecarbothioamide dissolved in 150 ml of a soda solution at 1.5M. The reaction medium is maintained under stirring at ambient temperature for 24 hours, then the beige precipitate formed is filtered, washed with water and dried (6.8 g; yield=66%). The mother liquors can be extracted with 3 times 75 ml of dichloromethane, then the organic phases are washed with a saturated solution of sodium chloride. After concentration under reduced pressure, the residue obtained can be purified on a silica column (eluent: ethyl acetate/heptane:1/3) to provide an additional 2 g of expected product (overall yield=86%). Melting point: 136-138° C.

NMR ¹H (DMSO d6, 400 MHz, δ): 7.65 (m, 1H, arom. H); 7.36-7.31 (m, 3H, arom. H); 6.75 (m, 1H, arom. H); 3.96 (s, 3H, CH₃); 3.87 (s, 3H, CH₃).

MS-LC: MH+=308.12; r.t.=11.48 min.

### 132.4) 2-(2,6-difluorophenyl)-5-methoxy-1,3-benzothiazole-4,7-dione

### 132.4.1) 2-(2,6-difluorophenyl)-5,7-dimethoxy-4-nitro-1,3-benzothiazole

A solution of 16 g (29.3 mmol; 3 equivalents) of cerium ammonium nitrate in 40 ml of water is added dropwise to 3 g (9.76 mmol) of 2-(2,6-difluorophenyl)-5,7-dimethoxy-1,3-benzothiazole in solution in 75 ml of ethyl acetate. The reaction mixture is maintained under stirring for 2 hours at ambient temperature, then washed with 3 times 20 ml of water. The organic phases are dried over magnesium sulphate, filtered then concentrated under reduced pressure. The residue is purified by chromatography on a silica column (eluent: ethyl acetate/heptane:3/7). Two fractions are separated:

0.3 g (yield 10%) of 2-(2,6-difluorophenyl)-5-methoxy-1,3-benzothiazole-4,7-dione is obtained in the form of a yellow powder.

MS-LC: MH+=308.08; r.t.=10 min.

1.5 g of 2-(2,6-difluorophenyl)-5,7-dimethoxy-4-nitro-1,3-benzothiazole (yield 45%) is obtained in the form of an orange powder.

NMR ¹H (DMSO d6, 400 MHz, δ): 7.72 (m, 1H, arom. H); 7.38 (m, 2H, arom. H); 7.11 (m, 1H, arom. H); 4.12 (s, 3H, CH₃); 4.07 (s, 3H, CH₃).

MS-LC: MH+=353.05; r.t.=11.30 min.

### 132.4.2) 2-(2,6-difluorophenyl}-5,7-dimethoxy-1,3-benzothiazol-4-amine

230 mg (0.65 mmol) of intermediate 132.4.1 in solution in 15 ml of concentrated hydrochloric acid is reacted with 0.5 g (2.2 mmol; 3.4 equivalents) of dihydrated tin chloride in 5 ml of water. The reaction mixture is maintained under stirring for 2 hours at 50° C., then after returning to ambient temperature, is poured onto ice before neutralizing with a 5 M soda solution. The product is then extracted with 3 times 15 ml of dichloromethane, the organic phases are combined, washed with a saturated solution of sodium chloride, dried over magnesium sulphate, filtered then, after concentration under reduced pressure, the expected product is obtained in the form of a yellow oil. It is used in the following stage without other purification.

NMR ¹H (DMSO d6, 400 MHz, δ): 7.67 (m, 1H, arom. H); 7.34 (m, 2H, arom. H); 6.92 (s, 1H, arom. H); 3.91 (s, 3H, CH₃); 3.90 (s, 3H, CH₃).

MS-LC: MH+=323.10; r.t.=9.86 min.

### 132.4.3) 2-(2,6-difluorophenyl}-5-methoxy-1,3-benzothiazole-4,7-dione

A solution of 1.22 g of cerium ammonium nitrate (2.23 mmol, 2.1 equivalents) in 8 ml of water is added to 343 mg (1.06 mmol) of 2-(2,6-difluorophenyl)-5,7-dimethoxy-1,3-benzothiazol-4-amine in solution in 25 ml of ethyl acetate. The reaction mixture is maintained under vigorous stirring at ambient temperature for 1 hour 30 minutes then the organic phase is separated and washed with 3 times 20 ml of water, then dried over magnesium sulphate, filtered and the solvent is evaporated off under reduced pressure. The residue is purified by chromatography on a silica column (eluent: ethyl acetate/heptane:3/7) and 280 mg (yield=86%) of 2-(2,6-difluorophenyl)-5-methoxy-1,3-benzothiazole-4,7-dione is obtained in the form of a yellow powder.

NMR ¹H (DMSO d6, 400 MHz, δ): 7.72 (m, 1H, arom. H); 7.39 (m, 2H. arom. H); 6.32 (s, 1H, CH); 3.88 (s, 3H, CH₃).

MS-LC: MH+=308.05; r.t.=9.99 min.

### 132.5) 2-(2,6-difluorophenyl}-5-{[2-(dimethylamino}ethyl]amino)-1,3-benzothiazole-4,7-dione

104 ml (0.95 mmol; 1.5 equivalents) of N,N-dimethylethylenediamine is added to 195 mg of 2-(2,6-difluorophenyl)-5-methoxy-1,3-benzothiazole-4,7-dione in solution in 20 ml of anhydrous ethanol. The reaction mixture is stirred at 70° C. for 2 hours then the solvent is evaporated off under reduced pressure. The residue is purified on a silica column (eluent: 5% methanol in dichloromethane). 130 mg (yield=57%) of expected compound is obtained in the form of a red powder.

NMR ¹H (DMSO d6, 400 MHz, δ): 7.72 (m, 1H, arom. H); 7.52 (m, 1H, NH.); 7.38 (m, 2H, arom. H); 5.60 (s, 1H, CH); 3.28 (m, 2H, CH₂); 2.53 (m, 2H, CH₂); 2.20 (s, 6H, 2CH₃).

MS-LC: MH+=364.14; r.t.=7.85 min.

The compounds of Examples 133 to 138 are obtained in a similar manner to that described for Example 132, suitable acyl chlorides replacing 2,6-difluorobenzoyl chloride in the first stage and N-(2-aminoethyl)pyrrolidine replacing N,N-dimethylethylenediamine in the last stage for Examples 134, 136 and 138.

### Example 133

### 2-(2,5-dichlorothien-3-yl)-5-{[2-(dimethylamino)ethyl]amino}-1,3-benzothiazole-4,7-dione

### 133.1) 2,5-dichloro-N-(3,5-dimethoxyphenyl)thiophene-3-carboxamide

NMR ¹H (DMSO d6, 400 MHz, δ): 10.20 (s, 1H, NH); 7.47 (s, 1H, arom. H); 6.95 (s, 1H, arom. H); 6.27 (s, 1H, arom. H); 3.72 (s, 6H, 2CH₃).

MS-LC: MH+=332.01; r.t.=11.08 min.

### 133.2) 2,5-dichloro-N-(3,5-dimethoxyphenyl)thiophene-3-carbothioamide

NMR ¹H (DMSO d6, 400 MHz, δ): 11.96 (s, 1H, NH); 7.30 (s, 1H, arom. H); 7.25 (s, 1H, arom. H); 6.44 (s, 1H, arom. H); 3.74 (s, 6H, 2CH₃).

MS-LC: MH+=348.00; r.t.=11.55 min.

### 133.3) 2-(2,5-dichlorothien-3-yl)-5,7-dimethoxy-1,3-benzothiazole

NMR ¹H (DMSO d6, 400 MHz, δ): 7.72 (s, 1H, arom. H); 7.22 (s, 1H, arom. H); 6.73 (s, 1H, arom. H); 3.96 (s, 3H, CH₃); 3.86 (s, 3H, CH₃).

MS-LC: MH+=345.94; r.t.=12.77 min.

### 133.4) 2-(2, S-dichlorothien-3-yl)-5-methoxy-1,3-benzothiazole-4,7-dione

NMR ¹H (DMSO d6, 400 MHz, δ): 7.75 (s, 1H, arom. H); 6.31 (s, 1H, CH); 3.88 (s, 3H, CH³).

MS-LC: MH+=345.98; Lt.=11.52 min.

### 133.5) 2-(2,5-dichlorothien-3-yl)-5-{[2-(dimethylamino)ethyl]amino}-1,3-benzothiazole-4,7-dione

NMR ¹H (DMSO d6, 400 MHz, δ): 7.72 (s, 1H, arom. H); 7.51 (m, 1H, NH.); 5.58 (s, 1H, CH); 3.36 (m, 2H, CH₂); 2.54 (m, 2H, CH₂); 2.20 (s, 6H, 2CH₃).

MS-LC: MH+=402.06; r.t.=8.42 min.

### Example 134

### 2-(2,5-dichlorothien-3-yl)-5-[(2-pyrrolidin-1-ylethyl)amino]-1,3-benzothiazole-4,7-dione

MS-LC: MH+=427.97; Lt.=8.70 min.

### Example 135

### 5-{[2-(dimethylamino)ethyl]amino}-2-(4-fluorophenyl)-1,3-benzothiazole-4,7-dione

### 135.1) N-(3,5-dimethoxyphenyl)-4-fluorobenzamide

NMR ¹H (DMSO d6, 400 MHz, δ): 10.15 (s, 1H, NH); 8.01 (m, 2H, arom. H); 7.36 (m, 2H, arom. H); 7.05 (m, 2H, arom. H); 6.26 (s, 1H, arom. H); 3.73 (s, 6H, 2CH₃).

MS-LC: MH+=276.17; r.t.=10.07 min.

### 135.2) N-(3,5-dimethoxyphenyl)-4-fluorobenzenecarbothioamide

MS-LC: MH+=292.17; r.t.=10.72 min.

### 135.3) 2-(4-fluorophenyl)-5,7-dimethoxy-1,3-benzothiazole

NMR ¹H (DMSO d6, 400 MHz, δ): 8.11 (m, 2H, arom. H); 7.40 (m, 2H, arom. H); 7.22 (s, 1H, arom. H); 6.69 (s, 1H, arom. H); 3.95 (s, 3H, CH₃); 3.86 (s, 3H, CH₃).

MS-LC: MH+=290.07; r.t.=11.93 min.

### 135.4) 2-(4-fluorophenyl)-5-methoxy-1,3-benzothiazole-4,7-dione

NMR ¹H (DMSO d6, 400 MHz, δ): 8.15 (m, 2H, arom. H); 7.42 (m, 2H, arom. H); 6.28 (s, 1H, CH); 3.87 (s, 3H, CH₃).

MS-LC: MH+=290.14; r.t.=11.95 min.

### 135.5) 5-{[2-(dimethylamino)ethyl]amino}-2-(4-fluorophenyl)-1,3-benzothiazole-4,7-dione

NMR ¹H (DMSO d6, 400 MHz, δ): 8.11 (m, 2H, arom. H); 7.48 (m, 1H, NH); 7.41 (m, 2H, arom. H); 5.57 (s, 1H, CH); 3.26 (m, 2H, CH₂); 2.55 (m, 2H, CH₂); 2.22 (s, 6H, 2CH₃).

MS-LC: MH+=346.18; r.t.=8.01 min.

### Example 136

### 2-(4-fluorophenyl)-5-[(2-pyrrolidin-1-ylethyl)amino]-1,3-benzothiazole-4,7-dione

NMR ¹H (DMSO d6, 400 MHz, δ): 8.12 (m, 2H, arom. H); 7.58 (m, 1H, NH); 7.41 (m, 2H, arom. H); 5.55 (s, 1H, CH); 3.41 (m, 2H, CH₂); 2.69 (m, 2H, CH₂); 2.51 (m, 2H, CH₂); 2.44 (m, 2H, CH₂); 1.70 (m, 4H, 2CH₂).

MS-LC: MH+=372.19; r.t.=8.12 min.

### Example 137

### 2-(2-chloro-6-fluorophenyl)-5-{[2-(dimethylamino)ethyl]amino}-1,3-benzothiazole-4,7-dione

### 137.1) 2-chloro-N-(3,5-dimethoxyphenyl)-6-fluorobenzamide

NMR ¹H (DMSO d6, 400 MHz, δ): 10.69 (s, 1H, NH); 7.53 (m, 1H, arom. H); 7.43 (m, 1H, arom. H); 7.37 (m, 1H, arom. H); 6.93 (m, 2H, arom. H); 6.29 (s, 1H, arom. H); 3.72 (s, 6H, 2CH₃)

MS-LC: MH+=310.15; r.t.=10.11 min.

### 137.2) 2-chloro-N-(3,5-dimethoxyphenyl)-6-fluorobenzenecarbothioamide

NMR ¹H (DMSO d6, 400 MHz, δ): 7.41 (m, 2H, arom. H); 7.27 (m, 3H, arom. H); 6.46 (s, 1H, arom. H); 3.75 (s, 6H, 2CH₃).

MS-LC: MH+=326.09; r.t.=10.73 min.

### 137.3) 2-(2-chloro-6-fluorophenyl)-5,7-dimethoxy-1,3-benzothiazole

NMR ¹H (DMSO d6, 400 MHz, δ): 7.66 (m, 1H, arom. H); 7.56 (m, 1H, arom. H); 7.47 (m, 1H, arom. H); 7.30 (s, 1H, arom. H); 6.77 (s, 1H, arom. H); 3.96 (s, 3H, CH₃); 3.88 (s, 3H, CH₃).

MS-LC: MH+=324.03; r.t.=11.60 min.

### 137.4) 2-(2-chloro-6-fluorophenyl)-5-methoxy-1,3-benzothiazole-4,7-dione

NMR ¹H (DMSO d6, 400 MHz, δ): 7.69 (m, 1H, arom. H); 7.61 (m, 1H, arom. H); 7.52 (m, 1H, arom. H); 6.32 (s, 1H, CH); 3.88 (s, 3H, CH₃).

MS-LC: MH+=324.03; r.t.=9.23 min.

### 137.5) 2-(2-chloro-6-fluorophenyl)-5-{[2-(dimethylamino)ethyl]amino}-1,3-benzothiazole-4,7-dione

NMR ¹H (DMSO d6, 400 MHz, δ): 7.67 (s, 1H, arom. H); 7.59 (m, 1H, arom. H); 7.55 (m, 1H, NH.); 7.49 (m, 1H, arom. H); 5.61 (s, 1H, CH); 3.36 (m, 2H, CH₂); 2.54 (m, 2H, CH₂); 2.19 (s, 6H, 2CH₃).

MS-LC: MH+=380.10; r.t.=7.88 min.

### Example 138

### 2-(2-chloro-6-fluorophenyl)-5-[(2-pyrrolidin-1-ylethyl)amino]-1,3-benzothiazole-4,7-dione

MS-LC: MH+=406.10; r.t.=8.01 min.

### Pharmacological Study for Examples 1 to 131 of the Compounds of General Formula (I)

### Test Protocols

i) Measurement of the Phosphatase Activity of the Purified Cdc25C Recombinant Enzyme

The phosphatase activity of the MBP-Cdc25C protein is evaluated by dephosphorylation of 3-O-methylfluorescein-phosphate (OMFP) to 3-O-methylfluorescein (OMF) with determination of the fluorescence of the reaction product at 475 nm. This test allows identification of the inhibitors of cdc25 recombinant enzyme. The preparation of the fusion protein MBP-cdc25C is described in PCT Patent Application WO 01/44467.

The reaction is carried out in 384-well plate format in a final volume of 50 μL. The MBP-Cdc25C protein (prepared as described above) is stored in the following elution buffer: 20 mM Tris-HCl pH 7.4; 250 mM NaCl; 1 mM EDTA; 1 mM of dithiothreitol (DTT); 10 mM maltose. It is diluted to a concentration of 60 μM in the following reaction buffer: 50 mM Tris-HCl pH 8.2; 50 mM NaC1; 1 mM DTT; 20% glycerol. Measurement of the background noise is carried out with the buffer without addition of the enzyme. The products are tested at decreasing concentrations starting from 40 μM. The reaction is initiated by the addition of an OMFP solution at 500 μM final (prepared extemporaneously from a 12.5 mM stock solution in 100% DMSO (Sigma #M2629)). After 4 hours at 30° C. in a disposable 384-well plate, the fluorescence measured at OD 475 nm is read using a Victor² plate reader (EGG-Wallac). Determination of the 50% inhibitory concentration of the enzymatic reaction is calculated from three independent experiments. Only the values included in the linear part of the sigmoid are retained for linear regression analysis.

ii) Characterization of the Antiproliferative Activity:

By way of example, the effect of a treatment on two human cell lines Mia-Paca2 and DU 145 with the compounds of the examples described previously will be studied. The cell lines DU145 (human prostate cancer cells) and Mia-PaCa2 (human pancreas cancer cells) were acquired from the American Tissue Culture Collection (Rockville, Md., USA). The cells placed in 80 μl of Dulbecco's Modified Eagle's medium (Gibco-Brl, Cergy-Pontoise, France) completed with 10% foetal calf serum inactivated by heating (Gibco-Brl, Cergy-Pontoise, France), 50,000 units/l of penicillin and 50 mg/l of streptomycin (Gibco-Brl, Cergy-Pontoise, France), and 2 mM of glutamine (Gibco-Brl, Cergy-Pontoise, France) were seeded on a 96-well plate on day 0. The cells were treated on day 1 for 96 hours with increasing concentrations of each of the compounds to be tested up to 10 μM. At the end of this period, quantification of cell proliferation is evaluated by a calorimetric test based on the cleavage of the tetrazolium salt WST1 by the mitochondrial dehydrogenases in viable cells leading to the formation of formazan (Boehringer Mannheim, Meylan, France). These tests are carried out in duplicate with 8 determinations per concentration tested. For each compound to be tested, the values included in the linear part of the sigmoid were retained for a linear regression analysis and used to estimate the inhibitory concentration IC₅₀. The products are solubilized in dimethylsulphoxide (DMSO) at 10⁻² M and finally used in culture with 0.1% DMSO.

### Results of the Tests

a) The compounds of Examples 1 to 98, 101 to 104 and 107 to 115 have an IC₅₀ below or equal to 10 μM on the phosphatase activity of the purified Cdc25-C recombinant enzyme.

b) The compounds of Examples 1 to 9, 11, 14 to 34, 36 to 53, 55 to 58, 60 to 98 and 101 to 115 have an IC₅₀ below or equal to 10 μM on the cell proliferation of Mia-Paca2 lines.

c) The compounds of Examples 1 to 9, 11, 14 to 34, 36 to 53, 55 to 58, 60 to 98 and 101 to 115 have an IC₅₀ below or equal to 10 μM on the cell proliferation of DU-145 lines.

### Example of Compound of General Formula (IV)

### Preparation of [7-(2-(R)-amino-1-oxo-3-thiopropyl)-(S)-8-(cyclohexylmethyl)-2-phenyl-5,6,7,8-tetrahydro-imidazo-[1,2a]-pyrazine]dimer tetrahydrochloride

### Stage 1: [7-(2-(R)-t-butyloxycarbonylamino-1-oxo-3-thiopropyl)-(S)-8-(cyclohexylmethyl)-2-phenyl-5,6,7,8-tetrahydro-imidazo-[1,2a]-pyrazine]dimer

Condensation of 2 equivalents of (S)-8-(cyclohexylmethyl)-2-phenyl-5,6,7,8-tetrahydro-imidazo-[1,2a]-pyrazine (obtained according to the protocol described in PCT application WO 97/30053) with 1 equivalent of Boc-L-Cystine is carried out in dimethylformamide in the presence of HBTU and diisopropylethylamine. After completion of the reaction, the reaction medium is diluted with water and the product collected by filtration. Purification by chromatography on silica allows to isolate the product with a yield of 60%.

### Stage 2: [7-(2-(R)-amino-1-oxo-3-thiopropyl)-(S)-8-(cyclohexylmethyl)-2-phenyl-5,6,7,8-tetrahydro-imidazo-[1,2a]-pyrazine]dimer tetrahydrochloride

The product of stage 1 is dissolved in isopropanol. After cooling down at 0° C., an excess of a HCl solution in isopropanol is added dropwise to monitor the release rate of the gas formed by the Boc deprotection. After agitation one night at ambient temperature, the completion of the reaction induces in situ crystallization of the tetrahydrochloride. The reaction medium is then cooled down at 0° C. to complete crystallization. Filtration, washing of the crystals with isopropanol and drying under vacuum allows to isolate the expected product with a yield of 75%.

### Examples of Combinations According to the Invention

### A) Test of Cell Proliferation on the HT-29 Cells

The combinations presented as examples of combinations according to the invention can be tested with regard to their biological activity and the results of the combination compared with the results obtained for each of the compounds of the combination used separately. The protocol for the test used to obtain the results shown is described below:

Cell line

The HT-29 cell line (human colon cancer cells) were acquired from the American Tissue Culture Collection (Rockville, Md., USA).

### Measurement of In Vitro Cell Proliferation

The HT-29 cells (2000 cells/well) are cultured in 96-well plates. On day 0, these cells are seeded in 90 μl of Dulbeceo's modified Eagle medium (Gibeo-Brl, Cergy-Pontoise, France) completed with 10% foetal calf serum inactived by heating (Gibeo-Brl, Cergy-Pontoise, France), 50000 units/l of penicillin and 50 mg/l streptomycin (Gibeo-Brl, Cergy-Pontoise, France), and 2 mM of glutamine (Gibco-Brl, Cergy-Pontoise, France).

The cells were treated simultaneously with concentrations of two products individually or in combination on Day 1 and for 120 hours.

At the end of the of this period (D6), quantification of cell proliferation is evaluated by a colorimetric test based on the cleavage of the tetrazolium salt WST1 by mitochondrial dehydrogenases in living cells leading to the formation of formazan (Boehringer Mannheim, Meylan, France). These tests are carried out at least in duplicate with 4 determinations for each individual product and for each combination tested. This allows determination of the number of living cells at the end of each treatment.

### B) Combinations According to the Invention

### Combination 1:

Cdc25 phophatase inhibitor=5-{[2-(dimethylamino)ethyl]amino}-2-methyl-1,3-benzothiazole-4,7-dione hydrochloride (A1)

Combined anti-cancer agent=5-fluorouracil (B1)

The results in the cell proliferation test described above (expressed in % of surviving cells) for the above-mentioned compounds individually or in combination are set out in the table below.

| Compound A1 | | Compound B1 |
|---|---|---|
| (5 × 10⁻⁷ M) | Compound A1 (5 × 10⁻⁷ M) + | (2.5 × 10⁻⁶ M) |
| individually | compound B1 (2.5 × 10⁻⁶ M) | individually |
| 35 | 13 | 42 |

### Combination 2:

Cdc25 phophatase inhibitor=5-{[2-(dimethylamino)ethyl]amino}-2-methyl-1,3-benzothiazole-4,7-dione hydrochloride (A1)

Combined anti-cancer agent=mitomycin C (B2)

The results in the cell proliferation test described above (expressed in % of surviving cells) for the above-mentioned compounds individually or in combination are set out in the table below.

| Compound A1 | | Compound B2 |
|---|---|---|
| (2.5 × 10⁻⁷ M) | Compound A1 (2.5 × 10⁻⁷ M) + | (10⁻⁷ M) |
| individually | compound B2 (10⁻⁷ M) | individually |
| 91 | 36 | 49 |

### Combination 3:

Cdc25 phosphatase inhibitor=5-{[2-(dimethylamino)ethyl]amino}-2-methyl-1,3-benzothiazole-4,7-dione hydrochloride (A1)

Combined anti-cancer agent=taxol (B3)

The results in the cell proliferation test described above (expressed in % of surviving cells) for the above-mentioned compounds individually or in combination are set out in the table below.

| Compound A1 | | Compound B3 |
|---|---|---|
| (5 × 10⁻⁷ M) | Compound A1 (5 × 10⁻⁷ M) + | (5 × 10⁻⁹ M) |
| individually | compound B3 (5 × 10⁻⁹ M) | individually |
| 18 | 9 | 74 |

### Combination 4:

Cdc25 phosphatase inhibitor=5-{[2-(dimethylamino)ethyl]amino}-2-methyl-1,3-benzothiazole-4,7-dione hydrochloride (A1)

Combined anti-cancer agent=cisplatin (B4)

The results in the cell proliferation test described above (expressed in % of surviving cells) for the above-mentioned compounds individually or in combination are set out in the table below.

| Compound A1 | | Compound B4 |
|---|---|---|
| (5 × 10⁻⁷ M) | Compound A1 (5 × 10⁻⁷ M) + | (10⁻⁵ M) |
| individually | compound B4 (10⁻⁵ M) | individually |
| 31 | 11 | 33 |

### Combination 5:

Cdc25 phosphatase inhibitor=5-{[2-(dimethylamino)ethyl]amino}-2-methyl-1,3-benzothiazole-4,7-dione hydrochloride (A1)

Combined anti-cancer agent=methotrexate (B5)

The results in the cell proliferation test described above (expressed in % of surviving cells) for the above-mentioned compounds individually or in combination are set out in the table below.

| Compound A1 | | Compound B5 |
|---|---|---|
| (5 × 10⁻⁷ M) | Compound A1 (5 × 10⁻⁷ M) + | (5 × 10⁻⁸ M) |
| individually | compound B5 (5 × 10⁻⁸ M) | individually |
| 27 | 17 | 83 |

### Combination 6:

Cdc25 phosphatase inhibitor=5-{[2-(dimethylamino)ethyl]amino}-2-methyl-1,3-benzothiazole-4,7-dione hydrochloride (A1)

Combined anti-cancer agent=doxorubicin (B6)

The results in the cell proliferation test described above (expressed in % of surviving cells) for the above-mentioned compounds individually or in combination are set out in the table below.

| Compound A1 | | Compound B6 |
|---|---|---|
| (5 × 10⁻⁷ M) | Compound A1 (5 × 10⁻⁷ M) + | (5 × 10⁻⁷ M) |
| individually | compound B6 (5 × 10⁻⁷ M) | individually |
| 25 | 9 | 38 |

### Combination 7:

Cdc25 phosphatase inhibitor=5-{[2-(dimethylamino)ethyl]amino}-2-methyl-1,3-benzothiazole-4,7-dione hydrochloride (A1)

Combined anti-cancer agent=4-(2-bromophenyl)-1-(2-(1-((4-cyano-3-methoxy)phenylmethyl)imidazo-5-yl)-1-oxoethyl)-1,2-dihydro-8-fluoroimidazo[1,2a][1,4]-benzodiazepine (B7)

The results in the cell proliferation test described above (expressed in % of surviving cells) for the above-mentioned compounds individually or in combination are set out in the table below.

| Compound A1 | | Compound B7 |
|---|---|---|
| (5 × 10⁻⁷ M) | Compound A1 (5 × 10⁻⁷ M) + | (10⁻⁵ M) |
| individually | compound B7 (10⁻⁵ M) | individually |
| 51 | 14 | 92 |

### Combination 8:

Cdc25 phosphatase inhibitor=5-{[2-(dimethylamino)ethyl]amino}-2-methyl-1,3-benzothiazole-4,7-dione hydrochloride (A1)

Combined anti-cancer agent=8-bromo-2-(1R-isopropyl-2-hydroxyethylamino)-4-(3-fluorophenylmethylamino)-pyrazolo[1,5-a]-1,3,5-triazine (B8)

The results in the cell proliferation test described above (expressed in % of surviving cells) for the above-mentioned compounds individually or in combination are set out in the table below.

| Compound A1 | | Compound B8 |
|---|---|---|
| (5 × 10⁻⁷ M) | Compound A1 (5 × 10⁻⁷ M) + | (10⁻⁶ M) |
| individually | compound B8 (10⁻⁶ M) | individually |
| 47 | 6 | 69 |

### Combination 9:

Cdc25 phosphatase inhibitor=5-{[2-(dimethylamino)ethyl]amino}-2-methyl-1,3-benzothiazole-4,7-dione hydrochloride (A1)

Combined anti-cancer agent=(1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine tetrahydrochloride (B9)

The results in the cell proliferation test described above (expressed in % of surviving cells) for the above-mentioned compounds individually or in combination are set out in the table below.

| Compound A1 | | Compound B9 |
|---|---|---|
| (5 × 10⁻⁷ M) | Compound A1 (5 × 10⁻⁷ M) + | (10⁻⁵ M) |
| individually | compound B9 (10⁻⁵ M) | individually |
| 36 | 9 | 38 |

### Combination 10:

Cdc25 phosphatase inhibitor=5-{[2-(dimethylamino)ethyl]amino}-2-methyl-1,3-benzothiazole-4,7-dione hydrochloride (A1)

Combined anti-cancer agent=amsacrine (B10)

The results in the cell proliferation test described above (expressed in % of surviving cells) for the above-mentioned compounds individually or in combination are set out in the table below.

| Compound A1 | | Compound B10 |
|---|---|---|
| (5 × 10⁻⁷ M) | Compound A1 (5 × 10⁻⁷ M) + | (10⁻⁷ M) |
| individually | compound B10 (10⁻⁷ M) | individually |
| 42 | 39 | 89 |

### Combination 11:

Cdc25 phosphatase inhibitor=5-{[2-(dimethylamino)ethyl]amino}-2-methyl-1,3-benzothiazole-4,7-dione hydrochloride (A1)

Combined anti-cancer agent ═SN-38 (B11)

The results in the cell proliferation test described above (expressed in % of surviving cells) for the above-mentioned compounds individually or in combination are set out in the table below.

| Compound A1 | | Compound B11 |
|---|---|---|
| (5 × 10⁻⁷ M) | Compound A1 (5 × 10⁻⁷ M) + | (5 × 10⁻⁹ M) |
| individually | compound B11 (5 × 10⁻⁹ M) | individually |
| 44 | 7 | 98 |

### Combination 12:

Cdc25 phosphatase inhibitor=5-{[2-(dimethylamino)ethyl]amino}-2-methyl-1,3-benzothiazole-4,7-dione hydrochloride (A1)

Combined anti-cancer agent=diflomotecan (B12)

The results in the cell proliferation test described above (expressed in % of surviving cells) for the above-mentioned compounds individually or in combination are set out in the table below.

| Compound A1 | | Compound B12 |
|---|---|---|
| (5 × 10⁻⁷ M) | Compound A1 (5 × 10⁻⁷ M) + | (10⁻⁹ M) |
| individually | compound B12 (10⁻⁹ M) | individually |
| 33 | 22 | 83 |

### Combination 13:

Cdc25 phosphatase inhibitor=5-{[2-(dimethylamino)ethyl]amino}-2-methyl-1,3-benzothiazole-4,7-dione hydrochloride (A1)

Combined anti-cancer agent=BN-80927 or (+)-9-chloro-5-ethyl-5-hydroxy-10-methyl-12-(4-methylpiperidinomethyl)-4,5,13,15-tetrahydro-1H,3H-oxepino[3′,4′:6,7]indolizino[1,2-c]quinoline-3,15-dione hydrochloride (B13)

The results in the cell proliferation test described above (expressed in % of surviving cells) for the above-mentioned compounds individually or in combination are set out in the table below.

| Compound A1 | | Compound B13 |
|---|---|---|
| (5 × 10⁻⁷ M) | Compound A1 (5 × 10⁻⁷ M) + | (10⁻⁹ M) |
| individually | compound B13 (10⁻⁹ M) | individually |
| 43 | 32 | 91 |

### Combination 14:

Cdc25 phosphatase inhibitor=5-{[2-(dimethylamino)ethyl]amino}-2-methyl-1,3-benzothiazole-4,7-dione hydrochloride (A1)

Combined anti-cancer agent=roscovitine (B14)

The results in the cell proliferation test described above (expressed in % of surviving cells) for the above-mentioned compounds individually or in combination are set out in the table below.

| Compound A1 | | Compound B14 |
|---|---|---|
| (10⁻⁶ M) | Compound A1 (10⁻⁶ M) + | (5 × 10⁻⁵ M) |
| individually | compound B14 (5 × 10⁻⁵ M) | individually |
| 36 | 0 | 44 |

### Combination 15:

Cdc25 phosphatase inhibitor=menadione (A2)

Combined anti-cancer agent=roscovitine (B14)

The results in the cell proliferation test described above (expressed in % of surviving cells) for the above-mentioned compounds individually or in combination are set out in the table below.

| Compound A2 | | Compound B14 |
|---|---|---|
| (4 × 10⁻⁵ M) | Compound A2 (4 × 10⁻⁵ M) + | (5 × 10⁻⁵ M) |
| individually | compound B14 (5 × 10⁻⁵ M) | individually |
| 7 | 0 | 47 |

### Combination 16:

Cdc25 phosphatase inhibitor=menadione (A2)

Combined anti-cancer agent=8-bromo-2-(1R-isopropyl-2-hydroxyethylamino)-4-(3-fluorophenylmethylamino)-pyrazolo[1,5-a]-1,3,5-triazine (B8)

The results in the cell proliferation test described above (expressed in % of surviving cells) for the above-mentioned compounds individually or in combination are set out in the table below.

| Compound A2 | | |
|---|---|---|
| (4 × 10⁻⁵ M) | Compound A2 (4 × 10⁻⁵ M) + | Compound B8 (10⁻⁶ M) |
| individually | compound B8 (10⁻⁶ M) | individually |
| 33 | 4 | 17 |

### Combinations 17 to 19 Produce Results Similar to Those Observed for Combinations 11 to 13.

### Combination 17:

Cdc25 phosphatase inhibitor=2-methyl-5-[(2-pyrrolidin-1-ylethyl)amino]-1,3-benzothiazole-4,7-dione benzoate (A3)

Combined anti-cancer agent=diflomotecan (B12)

### Combination 18:

Cdc25 phosphatase inhibitor=2-methyl-5-[(2-piperidin-1-ylethyl)amino]-1,3-benzothiazole-4,7-dione benzoate (A4)

Combined anti-cancer agent=BN-80927 or (+)-9-chloro-5-ethyl-5-hydroxy-10-methyl-12-(4-methylpiperidinomethyl)-4,5,13,15-tetrahydro-1H,3H-oxepino[3′,4′:6,7]indolizino[1,2-c]quinoline-3,15-dione hydrochloride (B13)

### Combination 19:

Cdc25 phosphatase inhibitor=2-(2-chloro-6-fluorophenyl)-5-{[2-(dimethylamino)ethyl]amino}-1,3-benzothiazole-4,7-dione (A5)

Combined anti-cancer agent=diflomotecan (B12)

### Combinations 20 to 22 Produce Results Similar to Those Observed for Combination 8.

### Combination 20:

Cdc25 phosphatase inhibitor=2-methyl-5-[(2-pyrrolidin-1-ylethyl)amino]-1,3-benzothiazole-4,7-dione benzoate (A3)

Combined anti-cancer agent=8-bromo-4-[(3-pyridyl)methylamino]-2-methylthio-pyrazolo[1,5-a]-1,3,5-triazine (B15)

### Combination 21:

Cdc25 phosphatase inhibitor=2-methyl-5-[(2-piperidin-1-ylethyl)amino]-1,3-benzothiazole-4,7-dione benzoate (A4)

Combined anti-cancer agent=8-bromo-2-(1R-isopropyl-2-hydroxyethylamino)-4-(3-fluorophenylmethylamino)-pyrazolo[1,5-a]-1,3,5-triazine (B8)

### Combination 22:

Cdc25 phosphatase inhibitor=2-(2-chloro-6-fluorophenyl)-5-{[2-(dimethylamino)ethyl]amino}-1,3-benzothiazole-4,7-dione (A5)

Combined anti-cancer agent=8-bromo-2-(1R-isopropyl-2-hydroxyethylamino)-4-(3-pyridylmethylamino)pyrazolo[1,5-a]-1,3,5-triazine (B16)

### Combination 23:

Cdc25 phosphatase inhibitor=2-methyl-5-[(2-pyrrolidin-1-ylethyl)amino]-1,3-benzothiazole-4,7-dione benzoate (A3)

Combined anti-cancer agent=taxol (B3)

The results in the cell proliferation test described above (expressed in % of surviving cells) for the above-mentioned compounds individually or in combination are set out in the table below.

| Compound A3 | Compound A3 | Compound |
|---|---|---|
| (1.25 × 10⁻⁷ M) | (1.25 × 10⁻⁷ M) + | B3 (5 × 10⁻⁹ M) |
| individually | compound B3 (5 × 10⁻⁹ M) | individually |
| 91 | 32 | 54 |

### Combination 24:

Cdc25 phosphatase inhibitor=2-methyl-5-[(2-pyrrolidin-1-ylethyl)amino]-1,3-benzothiazole-4,7-dione benzoate (A3)

Combined anti-cancer agent ═SN-38 (B11)

The results in the cell proliferation test described above (expressed in % of surviving cells) for the above-mentioned compounds individually or in combination are set out in the table below.

| Compound A3 | Compound | |
|---|---|---|
| (2.5 × 10⁻⁷ M) | A3 (2.5 × 10⁻⁷ M) + | Compound B11 (10⁻⁸ M) |
| individually | compound B11 (10⁻⁸ M) | individually |
| 75 | 40 | 73 |

### Combination 25:

Cdc25 phosphatase inhibitor=2-methyl-5-[(2-pyrrolidin-1-ylethyl)amino]-1,3-benzothiazole-4,7-dione benzoate (A3)

Combined anti-cancer agent=mitomycin C (B2)

The results in the cell proliferation test described above (expressed in % of surviving cells) for the above-mentioned compounds individually or in combination are set out in the table below.

| Compound A3 | Compound A3 | |
|---|---|---|
| (2.5 × 10⁻⁷ M) | (2.5 × 10⁻⁷ M) + | Compound B2 (5 × 10⁻⁸ M) |
| individually | compound B2 (5 × 10⁻⁸ M) | individually |
| 84 | 55 | 82 |

### Combination 26:

Cdc25 phosphatase inhibitor=2-methyl-5-(2-pyrrolidin-1-ylethyl)amino]-1,3-benzothiazole-4,7-dione benzoate (A3)

Combined anti-cancer agent=doxorubicin (B6)

The results in the cell proliferation test described above (expressed in % of surviving cells) for the above-mentioned compounds individually or in combination are set out in the table below.

| Compound A3 | Compound | |
|---|---|---|
| (2.5 × 10⁻⁷ M) | A3 (2.5 × 10⁻⁷ M) + | Compound B6 (10⁻⁷ M) |
| individually | compound B6 (10⁻⁷ M) | individually |
| 73 | 58 | 75 |

### Combination 27:

Cdc25 phosphatase inhibitor=2-methyl-5-[(2-pyrrolidin-1-ylethyl)amino]-1,3-benzothiazole-4,7-dione benzoate (A3)

Combined anti-cancer agent=8-bromo-2-(1R-isopropyl-2-hydroxyethylamino)-4-(3-fluorophenylmethylamino)-pyrazolo[1,5-a]-1,3,5-triazine (B8)

The results in the cell proliferation test described above (expressed in % of surviving cells) for the above-mentioned compounds individually or in combination are set out in the table below.

| Compound A3 | Compound | |
|---|---|---|
| (1.25 × 10⁻⁷ M) | A3 (1.25 × 10⁻⁷ M) + | Compound B8 (10⁻⁶ M) |
| individually | compound B8 (10⁻⁶ M) | individually |
| 89 | 12 | 21 |

### Combination 28:

Cdc25 phosphatase inhibitor=2-methyl-5-[(2-pyrrolidin-1-ylethyl)amino]-1,3-benzothiazole-4,7-dione benzoate (A3)

Combined anti-cancer agent=cisplatin (B4)

The results in the cell proliferation test described above (expressed in % of surviving cells) for the above-mentioned compounds individually or in combination are set out in the table below.

| Compound A3 | Compound | |
|---|---|---|
| (2.5 × 10⁻⁷ M) | A3 (2.5 × 10⁻⁷ M) + | Compound B4 (10⁻⁵ M) |
| individually | compound B4 (10⁻⁵ M) | individually |
| 78 | 36 | 75 |

### Combination 29:

Cdc25 phosphatase inhibitor=2-methyl-5-[(2-piperidin-1-ylethyl)amino]-1,3-benzothiazole-4,7-dione benzoate (A4)

Combined anti-cancer agent=taxol (B3)

The results in the cell proliferation test described above (expressed in % of surviving cells) for the above-mentioned compounds individually or in combination are set out in the table below.

| Compound A4 | Compound A4 | |
|---|---|---|
| (2.5 × 10⁻⁷ M) | (2.5 × 10⁻⁷ M) + | Compound B3 (5 × 10⁻⁹ M) |
| individually | compound B3 (5 × 10⁻⁹ M) | individually |
| 94 | 33 | 51 |

### Combination 30:

Cdc25 phosphatase inhibitor=2-methyl-5-[(2-piperidin-1-ylethyl)amino]-1,3-benzothiazole-4,7-dione benzoate (A4)

Combined anti-cancer agent ═SN-38 (B11)

The results in the cell proliferation test described above (expressed in % of surviving cells) for the above-mentioned compounds individually or in combination are set out in the table below.

| Compound A4 | | |
|---|---|---|
| (5 × 10⁻⁷ M) | Compound A4 (5 × 10⁻⁷ M) + | Compound B11 (10⁻⁸ M) |
| individually | compound B11 (10⁻⁸ M) | individually |
| 78 | 42 | 67 |

### Combination 31:

Cdc25 phosphatase inhibitor=2-methyl-5-[(2-piperidin-1-ylethyl)amino]-1,3-benzothiazole-4,7-dione benzoate (A4)

Combined anti-cancer agent=diflomotecan (B12)

The results in the cell proliferation test described above (expressed in % of surviving cells) for the above-mentioned compounds individually or in combination are set out in the table below.

| Compound A4 | | |
|---|---|---|
| (5 × 10⁻⁷ M) | Compound A4 (5 × 10⁻⁷ M) + | Compound B12 (10⁻⁹ M) |
| individually | compound B12 (10⁻⁹ M) | individually |
| 79 | 58 | 89 |

### Combination 32:

Cdc25 phosphatase inhibitor=2-methyl-5-(2-piperidin-1-ylethyl)amino]-1,3-benzothiazole-4,7-dione benzoate (A4)

Combined anti-cancer agent=mitomycin C (B2)

The results in the cell proliferation test described above (expressed in % of surviving cells) for the above-mentioned compounds individually or in combination are set out in the table below.

| Compound A4 | Compound | |
|---|---|---|
| (5 × 10⁻⁷ M) | A4 (5 × 10⁻⁷ M) + | Compound B2 (5 × 10⁻⁸ M) |
| individually | compound B2 (5 × 10⁻⁸ M) | individually |
| 74 | 83 | 88 |

### Combination 33:

Cdc25 phosphatase inhibitor=2-methyl-5-(2-piperidin-1-ylethyl)amino]-1,3-benzothiazole-4,7-dione benzoate (A4)

Combined anti-cancer agent=mitomycin C (B2)

The results in the cell proliferation test described above (expressed in % of surviving cells) for the above-mentioned compounds individually or in combination are set out in the table below.

| Compound A4 | Compound A4 | |
|---|---|---|
| (5 × 10⁻⁷ M) | (5 × 10⁻⁷ M) + | Compound B2 (5 × 10⁻⁸ M) |
| individually | compound B2 (5 × 10⁻⁸ M) | individually |
| 74 | 83 | 88 |

### Combination 34:

Cdc25 phosphatase inhibitor=2-methyl-5-[(2-piperidin-1-ylethyl)amino]-1,3-benzothiazole-4,7-dione benzoate (A4)

Combined anti-cancer agent=5-fluorouracil (B1)

The results in the cell proliferation test described above (expressed in % of surviving cells) for the above-mentioned compounds individually or in combination are set out in the table below.

| Compound A4 | | Compound B1 |
|---|---|---|
| (5 × 10⁻⁷ M) | Compound A4 (5 × 10⁻⁷ M) + | (2.5 × 10⁻⁶ M) |
| individually | compound B1 (2.5 × 10⁻⁶ M) | individually |
| 75 | 34 | 51 |

### Combination 35:

Cdc25 phosphatase inhibitor=2-methyl-5-[(2-piperidin-1-ylethyl)amino]-1,3-benzothiazole-4,7-dione benzoate (A4)

Combined anti-cancer agent=cisplatin (B4)

The results in the cell proliferation test described above (expressed in % of surviving cells) for the above-mentioned compounds individually or in combination are set out in the table below.

| Compound A4 | | |
|---|---|---|
| (5 × 10⁻⁷ M) | Compound A4 (5 × 10⁻⁷ M) + | Compound B4 (10⁻⁵ M) |
| individually | compound B4 (10⁻⁵ M) | individually |
| 71 | 43 | 85 |

### Combination 36:

Cdc25 phosphatase inhibitor=2-methyl-5-[(2-piperidin-1-ylethyl)amino]-1,3-benzothiazole-4,7-dione benzoate (A4)

Combined anti-cancer agent=(1R)-1-[({(2R)-2-amino-3-[(8S)-8-(cyclohexylmethyl)2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine tetrahydrochloride (B9)

The results in the cell proliferation test described above (expressed in % of surviving cells) for the above-mentioned compounds individually or in combination are set out in the table below.

| Compound A4 | | Compound B9 |
|---|---|---|
| (5 × 10-⁷ M) | Compound A4 (5 × 10-⁷ M) + | (5 × 10-⁶ M) |
| individually | compound B9 (5 × 10-⁶ M) | individually |
| 78 | 51 | 80 |

1-23. (canceled)

24. A pharmaceutical compound comprising:
(1R)— 1-[({(2R)-2-amino-3-[(8S)-8-(cyclohexylmethyl)-2-phenyl-5,6-dihydroimidazo[1,2-a]pyrazin-7-(8H)-yl]-3-oxopropyl}dithio)methyl]-2-[(8S)-8-(cyclohexylmethyl)-2-phenyl-5,6-dihydroimidazo[1,2-a]pyrazin-7(8H)-yl]-2-oxoethylamine, or
a pharmaceutically acceptable salt thereof.

25. The compound according to claim 24, wherein the compound is
(1R)— 1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)—-yl]-2-oxoethylamine tetrahydrochloride.

26. A process for making a salt, comprising the following steps:
1) reacting approximately 2 equivalents of (8S)-8-(cyclohexylmethyl)-2-phenyl-5,6,7,8-tetrahydroimidazo[1,2-a]pyrazine with approximately one equivalent of Boc-Cys-Cys-Boc in a polar aprotic solvent; and
2) reacting in a lower alcohol the disulphide derivative obtained after stage 1) with an excess of hydrochloric acid in solution in a lower alcohol.

27. The process of claim 26, wherein the salt is
(1R)-1-[({(2R)-2-amino-3-[(8S)-8-(cyclohexylmethyl)-2-phenyl-5,6-dihydroimidazo[1,2-a]pyrazin-7(8H)-yl]-3-oxopropyl}dithio)methyl]-2-[(8S)-8-(cyclohexylmethyl)-2-phenyl-5,6-dihydroimidazo[1,2-a]pyrazin-7(8H)—-yl]-2-oxoethylamine tetrahydrochloride.

28. A method of treating cancer or pain in a warm blooded animal comprising administering the compound of claim 24 to the warm blooded animal in need thereof.

29. The method of claim 29, wherein the cancer to be treated is cancer of the breast, lymphoma, cancers of the head and the neck, cancer of the colon, cancer of the prostrate or cancer of the pancreas.
